(19) United States
(12) Patent Application Publication
(10) Pub. No.: US 2013/0315825 A1
(43) Pub. Date: Nov. 28, 2013

(54) TRICYCLIC HETEROAROMATIC COMPOUNDS AS ALPHA-SYNUCLEIN LIGANDS

(71) Applicants: Zhude Tu, Frontenac, MO (US); Robert Mach, Eureka, MO (US); Lihai Yu, St. Louis, MO (US); Paul Kotzbauer, St. Louis, MO (US)

(72) Inventors: Zhude Tu, Frontenac, MO (US); Robert Mach, Eureka, MO (US); Lihai Yu, St. Louis, MO (US); Paul Kotzbauer, St. Louis, MO (US)

(73) Assignee: Washington University, St. Louis, MO (US)

(21) Appl. No.: 13/887,171

(22) Filed: May 3, 2013

Related U.S. Application Data
(60) Provisional application No. 61/642,025, filed on May 3, 2012.

(51) Int. Cl. C07D 279/20; A61K 51/04; C07D 241/46; C07D 513/04; C07D 265/38

(52) U.S. Cl. CPC C07D 279/20; C07D 513/04; C07D 265/38; C07D 241/46; A61K 51/0465
USPC 424/1.81; 544/35; 514/224.8; 544/37; 544/38; 544/39; 544/34; 544/102; 514/229.8; 544/347; 544/348; 514/250; 436/501; 435/7.92; 424/1.85; 424/1.89; 514/226.2; 514/224.5

(57) ABSTRACT

Derivatives of phenothiazine, phenoxazine, and phenazine compounds and their use as α-synuclein ligands are described. Also described are methods of using these compounds and their radiolabeled analogs for the detection, monitoring, and treatment of synucleinopathies, including Parkinson's disease.

### REFERENCE TO RELATED APPLICATIONS

This application claims the benefit of U.S. Provisional Application Ser. No. 61/642,025, filed May 3, 2012, the entire disclosure of which is incorporated herein by reference.

### GOVERNMENT LICENSE RIGHTS

This invention was made with Government support under grants NS061025, NS075527, and MH092797 awarded by the National Institutes of Health and grants NS075321, NS058714, and NS41509 (JSP) awarded by the National Institute of Neurological Disorders and Stroke (NINDS/NIH). The Government has certain rights in the invention.

### FIELD OF THE INVENTION

The present invention generally relates to derivatives of phenothiazine, phenoxazine, and phenazine compounds and their use as α-synuclein ligands. The invention further relates methods of using these compounds and their radiolabeled analogs for the detection and treatment of synucleinopathies, including Parkinson's disease (PD).

### BACKGROUND OF THE INVENTION

Neurodegenerative diseases such as Alzheimer's disease, Parkinson's disease (PD), Huntington's disease, amyotrophic lateral sclerosis and prion diseases are debilitating diseases which affect cognition and/or muscle control. These diseases are a subset of protein misfolding diseases. Protein folding is an essential process for protein function in all organisms, and conditions that disrupt protein folding present a threat to cell viability. In some cases the disease arises because a specific protein is no longer functional when adopting a misfolded state. In other diseases, the pathological state originates because misfolding occurs concomitantly with aggregation, and the underlying aggregates are detrimental.

Even though neurodegenerative diseases such as Alzheimer's and Parkinson's are caused by different proteins, both involve the accumulation of insoluble fibrous protein deposits, called amyloids. For example, PD, Dementia with Lewy Bodies (DLB), and multiple system atrophy (MSA), which are collectively referred to as “synucleinopathies,” have been linked to the accumulation of aggregated forms of the α-synuclein protein in neurons in the brain. As the primary neuropathologic change of PD, the degeneration of dopaminergic neurons occurred in the substantia nigra, as well as Lewy bodies (LB) and Lewy neurites (LN). To date, the pathogenic mechanism of PD has not been fully discovered.

α-Synuclein is a presynaptic terminal protein that consists of 140-amino acid protein that plays an important function in the central nervous system including synaptic vesicle recycling and synthesis, vesicular storage, and neurotransmitter release. It is specifically upregulated in a discrete population of presynaptic terminals of the brain during acquisition-related synaptic rearrangement. α-Synuclein naturally exists in a highly soluble, unfolded state. Recent evidence suggests that filamentous aggregates of α-synuclein accumulate at the pre-synaptic membrane and trigger synapse dysfunction and neuronal cell death in synucleinopathies, and may be the cause of Parkinson's and DLB. α-Synuclein aggregation has been identified by antibody-immunohistological studies as the major component of Lewy bodies, which are microscopic protein deposits in deteriorating nerve cells. Accumulation of misfolded, fibrillar α-synuclein in Lewy bodies (LB) and Lewy neurites (LN) considered a hallmark of PD.

The diagnosis of PD is mainly based on the clinical symptoms such as rest tremor, bradykinesia, and rigidity. The current treatment for PD is to slow the disease progression and minimize the disease symptoms in the patients. Therefore, a method of diagnosing PD in the very early stage can greatly help the physicians to design the therapy accordingly, and to slow the disease progression.

Since the conversion of a small number of soluble peptides and proteins into insoluble filaments is believed to be the central event in the pathologies of most neurodegenerative diseases, many strategies are aimed at inhibiting filament formation and at promoting filament clearance. In 2006, Masuda investigated the effects of 79 compounds belonging to 12 different chemical classes including polyphenols, phenothiazines, polyene macrolides, porphyrins, and rifamycins on Aβ, α-synuclein, and tau filament formation [1]. Polyphenols were shown to be a major class of compounds for α-synuclein inhibition, with fourteen of the tested compounds having IC₅₀ values<10.

Phenothiazine and certain derivatives such as methylene blue (i.e., 3,7-tetramethyldiaminophenothiazinium chloride) have been employed in a variety of applications including artificial dyes, anthelmintics, and therapeutic agents [2]. N-substituted phenothiazines and 3,7-diamino-substituted phenothiazines have been used as antihistamines, sedatives, and antipsychotics. Further, some N-substituted phenothiazines, such as N-(3-chloro-10H-phenothiazin-10-yl)-3-(dimethylamino)propanamide and N-(2-(10H-phenothiazin-10-yl)ethyl)-4-methylpiperazin-1-amine have been proposed for the therapy of neurodegenerative diseases by protecting the dopaminergic neurons against oxidative stress [3]. Some phenazines have been used in the production of artificial dyes.

Despite current efforts, there exists a need for improved diagnostic methods for identifying aggregations of misfolded proteins, including α-synuclein for early detection and ongoing monitoring of PD in subjects. Further, there exists a need for compounds that have a high affinity and selectivity for aggregated α-synuclein and compounds that inhibit the aggregation of α-synuclein for treating synucleinopathies such as PD.

### SUMMARY OF THE INVENTION

One aspect of the present invention is directed to tricyclic heteroaromatic compounds of Formula I

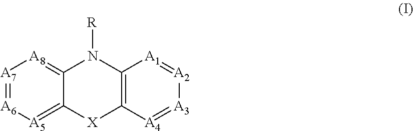

wherein X is oxygen, sulfur, or N—R;
each R is independently hydrogen, alkyl, or acyl;
A₁ is C—R₁ or nitrogen;
A₂ is C—R₂ or nitrogen;
A₃ is C—R₃ or nitrogen;
A₄ is C—R₄ or nitrogen;
A₅ is C—R₅ or nitrogen;
A₆ is C—R₆ or nitrogen;
A₇ is C—R₇ or nitrogen;
A₈ is C—R₈ or nitrogen; and
R₁, R₂, R₃, R₄, R₅, R₆, R₇, and R₈ are each independently hydrogen, halo, hydroxy, substituted or unsubstituted alkyl, substituted or unsubstituted alkoxy, cyano, nitro, amino, alkylamino, or dialkylamino; or a pharmaceutically acceptable salt thereof.

Another aspect of the present invention is directed to compounds of Formula I that are radiolabeled, for example with an isotope useful for positron emission tomography. In other aspects, the present invention is directed to a method for diagnosing or monitoring a synucleinopathy in a human subject comprising administering a radiolabeled compound of Formula I to the human subject; and imaging the subject's brain by positron emission tomography.

A further aspect of the invention is the use of a compound of Formula I for the treatment of synucleinopathies such as Parkinson's disease, Dementia with Lewy bodies, or multiple system atrophy.

Other aspects of the invention will be in part apparent and in part pointed out hereinafter.

### BRIEF DESCRIPTION OF THE DRAWINGS

FIG. 1 shows the reaction scheme for Compounds 11a, 11e, SIL5, SIL3B, and SIL22.

FIG. 2 shows the reaction scheme for Compounds 12, 13a-13c, 14a-14b, 15, 16c, SIL23, and SIL26.

FIG. 3 shows the reaction scheme for Compounds TZ-2-33, TZ-2-39, TZ-2-45, TZ-2-48, TZ-2-52, TZ-2-54, TZ-2-65, and TZ-2-69.

FIG. 4 shows the reaction scheme for Compounds TZ5B-71, TZ5B-79-1-1, TZ5B-95-1, TZ5B-145-2, TZ5B-159-1, TZ10-1-2, and TZ10-27-1.

FIG. 5(a) shows the reaction scheme for Compounds TZ16-147-2 and TZ16-147-3.

FIG. 5(b) shows the reaction scheme for Compound TZ16-133-2.

FIG. 6(a) is the fluorescence emission spectra of ThT in buffer alone, in the presence of α-synuclein monomer, and in the presence of α-synuclein fibrils at λₑₘ=440 nm.

FIG. 6(b) is the saturation curve of ThT for α-synuclein fibrils at difference incubation times.

FIG. 7 is a plot of the inhibition curve for Compound 6 in the ThT competitive binding assay

FIG. 8 is a plot of the inhibition curve for Compound 11a in the ThT competitive binding assay.

FIG. 9 is a plot of the inhibition curve for Compound SIL5 in the ThT competitive binding assay.

FIG. 10 is a plot of the inhibition curve for Compound SIL3B in the ThT competitive binding assay.

FIG. 11 is a plot of the inhibition curve for Compound SIL22 in the ThT competitive binding assay.

FIG. 12 is a plot of the inhibition curve for Compound 11e in the ThT competitive binding assay.

FIG. 13 is a plot of the inhibition curve for Compound 12 in the ThT competitive binding assay.

FIG. 14 is a plot of the inhibition curve for Compound 13a in the ThT competitive binding assay.

FIG. 15 is a plot of the inhibition curve for Compound 13b in the ThT competitive binding assay.

FIG. 16 is a plot of the inhibition curve for Compound 13c in the ThT competitive binding assay.

FIG. 17 is a plot of the inhibition curve for Compound 14a in the ThT competitive binding assay.

FIG. 18 is a plot of the inhibition curve for Compound 14b in the ThT competitive binding assay.

FIG. 19 is a plot of the inhibition curve for Compound 15 in the ThT competitive binding assay.

FIG. 20 is a plot of the inhibition curve for Compound SIL26 in the ThT competitive binding assay.

FIG. 21 is a plot of the inhibition curve for Compound SIL23 in the ThT competitive binding assay.

FIG. 22 is a plot of the inhibition curve for Compound 16c in the ThT competitive binding assay.

FIG. 23 is the uv/vis absorbance spectrum for Compound SIL5 at the IC₅₀ concentration of 133 nM.

FIG. 24 is a representative plot of specific binding versus [¹²⁵I]SIL23 concentration.

FIG. 25 is a Scatchard analysis of the binding data shown in FIG. 24.

FIG. 26 is a plot of the amount of bound radioligand [¹²⁵I]SIL23 as a function of the concentration of unlabeled competitor ligand SIL22 in the incubation mixture containing α-synuclein fibrils.

FIG. 27 is a plot of the amount of bound radioligand [¹²⁵I]SIL23 as a function of the concentration of unlabeled competitor ligand SIL26 in the incubation mixture containing α-synuclein fibrils.

FIG. 28 is a plot of the amount of bound radioligand [¹²⁵I]SIL23 as a function of the concentration of unlabeled competitor ligand SIL3B in the incubation mixture containing α-synuclein fibrils.

FIG. 29 is a plot of the amount of bound radioligand [¹²⁵I]SIL23 as a function of the concentration of unlabeled competitor ligand SIL5 in the incubation mixture containing α-synuclein fibrils.

FIG. 30 is a plot of binding affinities of [¹²⁵I]SIL23 to Aβ fibrils.

FIG. 31 is a plot of binding affinities of [¹²⁵I]SIL23 to tau fibrils.

FIG. 32 is a representative plot of specific binding versus [¹²⁵I]SIL23 concentration in PD-Dementia #1 case.

FIG. 33 is a representative plot of specific binding versus [¹²⁵I]SIL23 concentration in PD-Dementia #2 case.

FIG. 34 is a representative plot of specific binding versus [¹²⁵I]SIL23 concentration in PD-Dementia #3 case.

FIG. 35 is a representative plot of specific binding versus [¹²⁵I]SIL23 concentration in PD-Dementia #4 case.

FIG. 36 is a representative plot of specific binding versus [¹²⁵I]SIL23 concentration Control #1.

FIG. 37 is a representative plot of specific binding versus [¹²⁵I]SIL23 concentration Control #2.

FIG. 38 is a representative plot of specific binding versus [¹²⁵I]SIL23 concentration Control #3.

FIG. 39 is a representative plot of specific binding versus [¹²⁵I]SIL23 concentration Control #4.

FIG. 40 is a representative syn1 western blot with SDS extracts from PD and control cases.

FIG. 41 is a quantitative syn303 western blot with SDS extracts from PD cases.

FIG. 42 is a plot with a correlation of Bₘₐₓ values for [¹²⁵I]SIL23 binding to levels of total insoluble α-synuclein quantified from the monomer band on western blot.

FIG. 43 is a plot with a correlation of Bₘₐₓ values for [¹²⁵I]SIL23 binding to levels of total insoluble α-synuclein quantified from monomer plus high molecular weight species on western blot.

FIG. 44 is a representative plots of specific binding versus [¹²⁵I]SIL23 concentration in mouse brain samples obtained from M83 mice with transgenic expression of human A53T α-synuclein.

FIG. 45 is a representative plots of specific binding versus [¹²⁵I]SIL23 concentration in mouse brain samples obtained from M7 mice with transgenic expression of human WT α-synuclein.

FIG. 46 shows the results of competitive binding studies of Aβ and tau fibrils with increasing concentrations of SIL22.

FIG. 47 shows the results of competitive binding studies of Aβ and tau fibrils with increasing concentrations of SIL26.

FIG. 48 shows the results of competitive binding studies of Aβ and tau fibrils with increasing concentrations of SIL23B.

FIG. 49 shows the results of competitive binding studies of Aβ and tau fibrils with increasing concentrations of SIL5.

FIG. 50 shows the results of the control cases in the competitive binding studies of Aβ and tau fibrils with increasing concentrations of SIL22.

FIG. 51 shows the results of the control cases in the competitive binding studies of Aβ and tau fibrils with increasing concentrations of SIL26.

FIG. 52 shows the results of the control cases in the competitive binding studies of Aβ and tau fibrils with increasing concentrations of SIL3B.

FIG. 53 shows the results of the control cases in the competitive binding studies of Aβ and tau fibrils with increasing concentrations of SIL5.

FIG. 54 is a representative plot of the correlation of Bₘₐₓ values for [¹²⁵I]SIL23 binding to levels of total insoluble α-synuclein quantified from ELISA (Pearson correlation coefficient R=0.98, p=0.0008).

FIG. 55 is a plot of the amount of bound radioligand [¹²⁵I]SIL23 as a function of the concentration of unlabeled competitor ligand SIL22 in homogenized insoluble fractions from human PD brain samples.

FIG. 56 is a plot of the amount of bound radioligand [¹²⁵I]SIL23 as a function of the concentration of unlabeled competitor ligand SIL26 in homogenized insoluble fractions from human PD brain samples.

FIG. 57 is a plot of the amount of bound radioligand [¹²⁵I]SIL23 as a function of the concentration of unlabeled competitor ligand SIL3B in homogenized insoluble fractions from human PD brain samples.

FIG. 58 is a plot of the amount of bound radioligand [¹²⁵I]SIL23 as a function of the concentration of unlabeled competitor ligand SIL5 in homogenized insoluble fractions from human PD brain samples.

FIG. 59 is a plot of the amount of bound radioligand [¹²⁵I]SIL23 as a function of the concentration of unlabeled competitor ligand ThT in an incubation mixture containing recombinant α-synuclein fibrils.

FIG. 60 is a plot of the amount of bound radioligand [¹²⁵I]SIL23 as a function of the concentration of unlabeled competitor ligand BGF227 in an incubation mixture containing recombinant α-synuclein fibrils.

FIG. 61 is a plot of the amount of bound radioligand [¹²⁵I]SIL23 as a function of the concentration of unlabeled competitor ligand chrysamine G in an incubation mixture containing recombinant α-synuclein fibrils.

FIG. 62 is a plot of the amount of bound radioligand [¹²⁵I]SIL23 as a function of the concentration of unlabeled competitor ligand PiB in an incubation mixture containing recombinant α-synuclein fibrils.

FIG. 63 is a plot of the amount of bound radioligand [¹²⁵I]SIL23 as a function of the concentration of unlabeled competitor ligand ThT in homogenized insoluble fractions from human PD brain samples.

FIG. 64 is a plot of the amount of bound radioligand [¹²⁵I]SIL23 as a function of the concentration of unlabeled competitor ligand BGF227 in homogenized insoluble fractions from human PD brain samples.

FIG. 65 is a plot of the amount of bound radioligand [¹²⁵I]SIL23 as a function of the concentration of unlabeled competitor ligand chrysamine G in homogenized insoluble fractions from human PD brain samples.

FIG. 66 is a plot of the amount of bound radioligand [¹²⁵I]SIL23 as a function of the concentration of unlabeled competitor ligand PiB in homogenized insoluble fractions from human PD brain samples.

FIG. 67 is a representative plot of specific binding versus [¹²⁵I]SIL23 concentration in crude phosphate buffered saline homogenates from human PD brain samples.

FIG. 68 is a Scatchard analysis of the binding data in FIG. 67.

FIG. 69 is a representative plot of specific binding versus [¹²⁵I]SIL23 concentration in control brain samples.

FIG. 70 Scatchard analysis for binding data in FIG. 69.

FIG. 71 is a bar graph of brain regional radioactivity uptake of [¹¹C]SIL5 in male SD rat brain (n=4).

FIG. 72 is a bar graph of brain regional radioactivity uptake of [¹⁸F]SIL26 in male SD rat brain (n=4).

FIG. 73 is a MicroPET scan of e brain of a nonhuman primate administered [¹¹C]SIL5.

FIG. 74 is a plot of [¹¹C]SIL5 washout over time in the brain of a nonhuman primate.

### DESCRIPTION OF THE INVENTION

Generally, the present invention is directed to tricyclic heteroaromatic compounds and methods of using these compounds. In particular, various tricyclic heteroaromatic compounds of the present invention are useful as α-synuclein ligands. The compounds possess an acceptable degree of binding affinity to α-synuclein fibrils which is useful for certain diagnostic methods for synucleinopathies such as PD.

In another aspect, the α-synuclein ligands of the present invention can be labeled with radionuclides such as carbon-11 and/or fluorine-18 to serve as Positron Emission Tomography (PET) probes for quantifying α-synuclein protein aggregation in the brain. The in vivo quantification of α-synuclein protein aggregation in patients would be useful not only for the early diagnosis of synucleinopathies such as PD, but also to monitor disease progression. Furthermore, some of these compounds may be useful as therapeutic agents for the inhibition of α-synuclein protein aggregation for the treatment of PD and related synucleinopathies.

As noted, fibrillar α-synuclein imaging may also be a highly useful marker for disease progression. The distribution patterns for pathological α-synuclein among autopsy cases with incidental LB disease and symptomatic PD indicate that disease progression is associated with an ascending, progressive involvement of multiple brain regions. A staging system has been developed, in which early stage cases are defined by involvement of olfactory nucleus, as well as select nuclei in the medulla and pons. Intermediate stages are defined by additional involvement of substantia nigra pars compacta, basal forebrain cholinergic nuclei, and select nuclei within the hypothalamus and amygdala. Late stages are defined by progressive involvement of neocortex. The link between disease progression and increasingly widespread involvement of multiple brain regions is supported by the association of neocortical LBs with the development of dementia in PD, which occurs in up to 80% of patients within 20 years after onset of motor symptoms. Longitudinal studies with an imaging tracer to quantify the amount and distribution of fibrillar α-synuclein in vivo would better define the natural disease course. This approach could also define the relative vulnerability of brain regions within the context of disease duration and establish correlations between the distribution of α-synuclein deposition and non-motor features of PD.

An effective diagnostic maker such as an α-synuclein imaging tracer would enable accurate enrollment of early stage PD patients into trials of therapeutic interventions targeting disease progression. If progressive accumulation of α-synuclein within individual regions or across multiple brain regions correlates with disease progression, particularly in early and intermediate disease stages, an α-synuclein imaging tracer could also greatly improve evaluation of therapeutic efficacy for candidate disease-modifying interventions.

Applicants have discovered that certain derivatives of phenothiazine, phenoxazine, and phenazine are useful as α-synuclein ligands. Phenothiazine is a tricyclic heteroaromatic compound containing nitrogen at the 10-position and sulfur at the 5-position. The positions on phenothiazine are numbered for naming purposes as shown below.

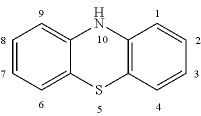 [SMILES: c1ccc2c(c1)Nc1ccccc1S2]

Phenoxazine is similar to phenothiazine, however oxygen is at the 5 position rather than sulfur. The positions on phenoxazine are numbered for naming purposes as shown below.

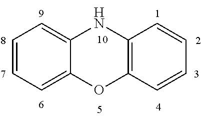 [SMILES: c1ccc2c(c1)Nc1ccccc1O2]

5,10-dihydrophenazine is also similar to phenothiazine, however N—H is at the 5 position rather than sulfur. The positions on phenazine are numbered for naming purposes as shown below.

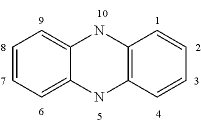 [SMILES: c1ccc2c(c1)Nc1ccccc1N2]

In accordance with the present invention, the tricyclic heteroaromatic compounds useful as α-synuclein ligands comprise compounds of Formula I:

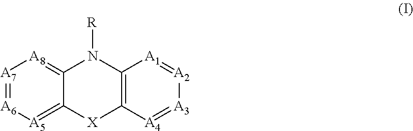

wherein X is oxygen, sulfur, or N—R; each R is independently hydrogen, alkyl, or acyl; A₁ is C—R₁ or nitrogen; A₂ is C—R₂ or nitrogen; A₃ is C—R₃ or nitrogen; A₄ is C—R₄ or nitrogen; A₅ is C—R₅ or nitrogen; A₆ is C—R₆ or nitrogen; A₇ is C—R₇ or nitrogen; A₈ is C—R₈ or nitrogen; and R₁, R₂, R₃, R₄, R₅, R₆, R₇, and R₈ are each independently hydrogen, halo, hydroxy, substituted or unsubstituted alkyl, substituted or unsubstituted alkoxy, cyano, nitro, amino, alkylamino, or dialkylamino; or a pharmaceutically acceptable salt thereof.

Unless otherwise indicated, the alkyl groups described herein are preferably lower alkyl containing from 1 to 20 carbon atoms in the principal chain. They may be straight or branched chain or cyclic. Also, unless otherwise indicated, the alkoxy groups described herein contain saturated or unsaturated, branched or unbranched carbon chains having from 1 to 20 carbon atoms in the principal chain.

In various embodiments, the tricyclic heteroaromatic compounds of Formula I are derivatives of phenothiazine (i.e., X is sulfur in Formula I). In other embodiments, the tricyclic heteroaromatic compounds of Formula I are derivatives of phenoxazine (i.e., X is oxygen in Formula I). In still further embodiments, the tricyclic heteroaromatic compounds of Formula I are derivatives of phenazine (i.e., X is N—R in Formula I).

In various embodiments, each R is independently hydrogen, C₁-C₆ alkyl, or C₁-C₆ acyl. Without being bound by theory, in some instances, selection of lower chain length substituents for R is believed to enhance the binding affinity and selectivity of the compound to α-synuclein. Accordingly, in certain embodiments, each R is independently hydrogen, C₁-C₄ alkyl, or C₁-C₄ acyl. In these and other embodiments, each R is independently hydrogen, methyl, or acetyl. In some embodiments, the tricyclic heteroaromatic compounds of Formula I are not N-substituted (i.e., R is hydrogen).

In various embodiments, one or more of A₁, A₂, A₃, A₄, A₅, A₆, A₇, or A₈ is nitrogen. In some embodiments, one of A₁, A₂, A₃, A₄, A₅, A₆, A₇, or A_{g} is nitrogen (e.g., either A₁ or A₃ is nitrogen). In various embodiments, either A₁ or A₃ is nitrogen and A₂ is C—R₂, A₄ is C—R₄, A₅ is C—R₅, A₆ is C—R₆, A₇ is C—R₇, and A_{g} is C—R₈. In other embodiments, A₁ is C—R₁₅ A₂ is C—R₂, A₃ is C—R₃, A₄ is C—R₄, A₅ is C—R₅, A₆ is C—R₆, A₇ is C—R₇, and A_{g} is C—R₈.

In various embodiments, R₁, R₂, R₃, R₄, R₅, R₆, R₇, and R₉ are each independently hydrogen, halo (e.g., fluoro, bromo, or iodo), hydroxy, substituted or unsubstituted C₁-C₆ alkyl, substituted or unsubstituted C₁-C₆ alkoxy, cyano, nitro, amino, C₁-C₆ alkylamino, or di-C₁-C₆ alkylamino. In these and other embodiments, R₁, R₂, R₃, R₄, R₅, R₆, R₇, and R₈ are each independently hydrogen, bromo, iodo, hydroxy, C₁-C₄ alkyl, C₁-C₄ haloalkyl, substituted or unsubstituted C₁-C₄ alkoxy, cyano, nitro, amino, C₁-C₄ alkylamino, or di-C₁-C₄ alkylamino. In some embodiments, R₁, R₂, R₃, R₄, R₅, R₆, R₇, and R₈ are each independently hydrogen, bromo, iodo, hydroxy, methyl, trifluoromethyl, methoxy, C₂-C₄ alkynloxy (e.g., —OCH₂C≡CH), halo substituted C₁-C₄ alkoxy (e.g., —OCH₂CH₂F), halo substituted C₂-C₄ alkenyloxy (e.g., —OCH₂CH═CHI or —OCH₂CH═CHBr), cyano, nitro, amino, methylamino, or dimethylamino.

In various embodiments, R₁, R₂, R₃, R₄, R₅, R₆, R₇, and R₉ are each independently hydrogen, bromo, iodo, hydroxy, methyl, trifluoromethyl, methoxy, propargyloxy (i.e., —OCH₂C≡CH), 2-fluoroethoxy (i.e., —OCH₂CH₂F), 3-iodoallyloxy (i.e., —OCH₂CH═CHI), 3-bromoallyloxy, cyano, nitro, amino, methylamino, or dimethylamino. In these and other embodiments, R₁, R₂, R₃, R₄, R₅, R₆, R₇, and R₈ are each independently hydrogen, methoxy, nitro, bromo, or iodo. In these and other embodiments, at least one of R₁, R₂, R₃, R₄, R₅, R₆, R₇, and R₈ is methoxy, nitro, bromo, or iodo. In various embodiments, at least one of R₁, R₂, R₃, R₄, R₅, R₆, R₇, and R₈ is methoxy. In some embodiments, at least one of R₁, R₂, R₃, R₄, R₅, R₆, R₇, and R₈ is nitro (e.g., one of R₁, R₃, or R₆ is nitro).

In various embodiments, R₃ and R₆ are each independently hydrogen, bromo, iodo, hydroxy, methyl, trifluoromethyl, methoxy, propargyloxy (i.e., —OCH₂C≡CH), fluoroethoxy (i.e., —OCH₂CH₂F), 3-iodoallyloxy (i.e., —OCH₂CH═CHI), cyano, nitro, amino, methylamino, or dimethylamino. In certain embodiments, R₃ and/or R₆ are methoxy. In certain embodiments R₃ and/or R₆ are methoxy. In certain embodiments, R₃ is nitro.

In various embodiments, at least one of R₁, R₂, R₃, R₄, R₅, R₆, R₇, and R₈ is nitro and at least one of R₁, R₂, R₃, R₄, R₅, R₆, R₇, and R₈ is methoxy. In other embodiments, at least one of R₁, R₂, R₃, R₄, R₅, R₆, R₇, and R₈ is nitro and at least one of R₁, R₂, R₃, R₄, R₅, R₆, R₇, and R₈ is bromo or iodo.

In various embodiments, the compound of Formula I is selected from the group consisting of:

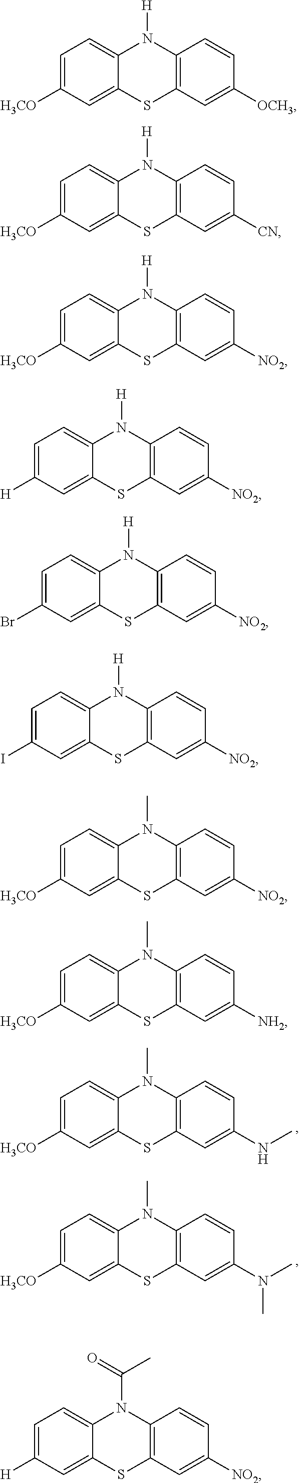

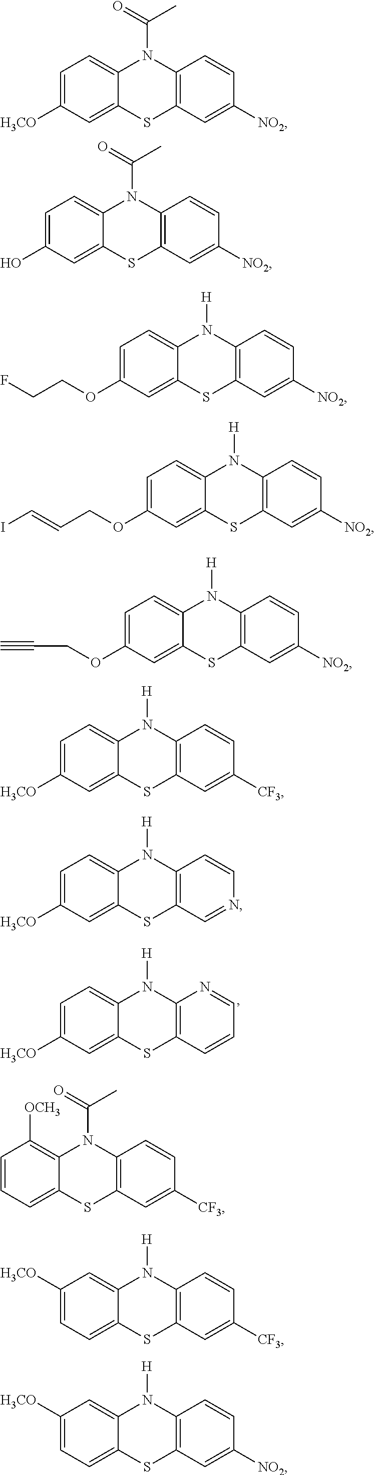

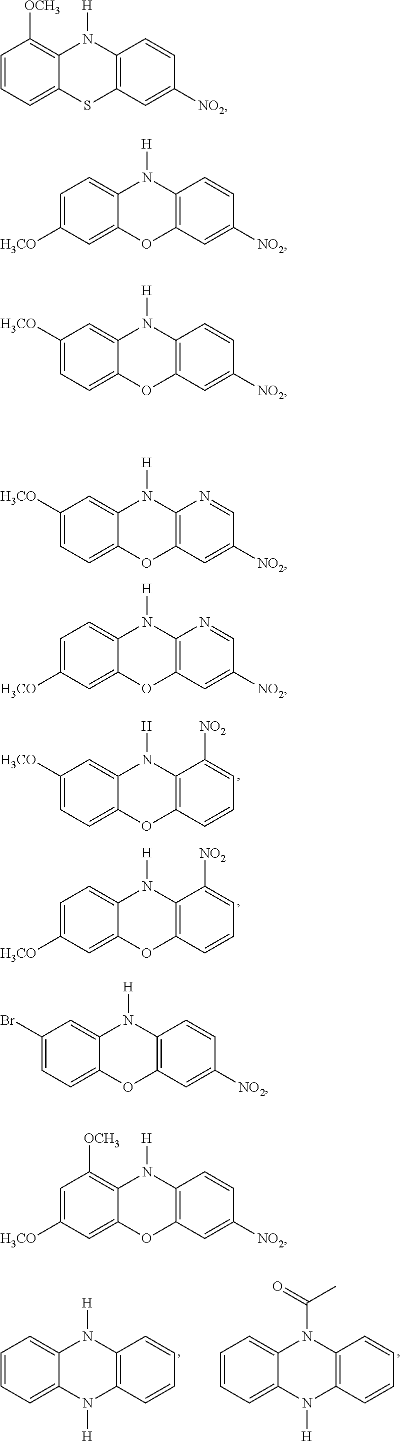

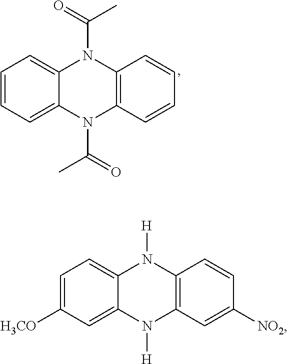 [SMILES: CC(=O)N1c2ccccc2N(C(C)=O)c2ccccc21 ; COc1ccc2c(c1)Nc1cc([N+](=O)[O-])ccc1N2]

and pharmaceutically acceptable salts thereof.

In general, various tricyclic heteroaromatic compounds of the present invention are α-synuclein ligands. The compounds possess an acceptable degree of binding affinity to α-synuclein fibrils which is useful for certain diagnostic and monitoring methods for synucleinopathies such as PD. The in vivo quantification of α-synuclein protein aggregation in patients is beneficial not only for the early diagnosis of synucleinopathies, but also for monitoring disease progression.

One diagnostic method that is suitable for use with the α-synuclein ligands of the present invention is positron emission tomography (PET). PET is known in the art of nuclear medicine imaging as a non-invasive imaging modality that can provide functional information of a living subject at molecular and cellular level. Also, PET is able to provide a non-invasive tool for diagnosing Alzheimer Disease in early stage when combining with [¹⁸F] Florbeapir, a radioligand of β-amyloid aggregates. PET utilizes biologically active molecules in micromolar or nanomolar concentrations that have been labeled with short-lived positron emitting isotopes. The physical characteristics of the isotopes and the molecular specificity of labeled molecules, combined with the high detection efficacy of modern PET scanners provides a sensitivity for in vivo measurements of indicator concentrations that is several orders of magnitude higher than with other imaging techniques.

In order to make measurements with PET, a biologically active tracer molecule tagged with a positron-emitting isotope is administered to a subject, for example, intravenously, orally or by inhalation. The subject is then scanned, and axial tomographic slices of regional cerebral tracer accumulation are obtained. This tracer accumulation can be related to cerebral metabolism, blood flow, or binding site concentrations by appropriate mathematical models. Thus, by using a small molecular PET radiotracer which has high affinity and selectivity to α-synuclein protein, the level of α-synuclein aggregation can be quantified. This approach not only improves the diagnostic accuracy of PD, but also provides a tool to monitor the progression of the disease and the efficacy of the treatment, and improve the understanding of disease progression.

Accordingly, the compounds of the present invention, such as those represented by Formula I, can be labeled with a radionuclide including, for example, carbon-11, nitrogen-13, oxygen-15, fluorine-18, bromine-76, iodine-123, and iodine-125 to serve as tracers for quantifying α-synuclein protein aggregation in the brain. In various embodiments, the compounds of Formula I are labeled with a radioactive halogen isotope selected from the group consisting of carbon 11, fluorine-18, iodine-123, and iodine-125. Methods known in the art for radiolabeling the compounds of the present invention may be used. See, for example, references [4] and [5], the contents of which are hereby incorporated herein by reference for all relevant purposes. Reagents having a radionuclide that may be used in the preparation radiolabeled compounds of the present invention include for example [¹¹C]CH₃I.

Further, in accordance with the present invention, methods for diagnosing or monitoring synucleinopathies are provided. In various embodiments, the method for diagnosing or monitoring a synucleinopathy in a human subject comprises administering a radiolabeled compound of Formula Ito the human subject; and imaging the subject's brain by positron emission tomography.

In accordance with the present invention, compounds potentially useful for treating synucleinopathies in a human subject in need thereof are provided. Accordingly, a method for treating synucleinopathies in a human subject in need thereof are provided comprises administering a therapeutically effective amount a compound of Formula Ito the human subject. In various embodiments, the synucleinopathy comprises PD, Dementia with Lewy Bodies, or multiple system atrophy.

In accordance with other aspects of the present invention, the compounds of present invention may be formulated in a suitable pharmaceutical delivery medium or vehicle. In various embodiments, the pharmaceutical delivery medium comprises an injectable comprising a compound of the present invention. In other embodiments, the pharmaceutical delivery medium comprises an oral vehicle comprising a compound of the present invention (e.g., capsule, pill, liquid, suspension, etc.).

A method for determining the binding affinity of a compound to α-synuclein is also provided. In some instances, test compounds do not significantly fluoresce such that their binding affinity to α-synuclein can be determined via direct fluorescence methodology. Accordingly, an indirect method for determining the binding affinity of a compound to α-synuclein is necessary. One indirect method comprises a competitive assay using the fluorescent dye Thioflavin T (ThT), which has the structure shown below.

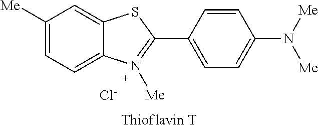

ThT is a benzothiazole dye that exhibits enhanced fluorescence upon binding to amyloid fibrils, and is used for the selective staining and identification of amyloid fibrils both in vitro and ex vivo. The changes in the fluorescent properties of ThT upon binding to amyloid fibrils include a shift in its excitation state and an increase in quantum yield. ThT in protic solvents principally absorbs at 340 nm with an emission maximum at 445 nm. Upon binding to amyloid fibrils, a peak at approximately 440 nm becomes dominant with the fluorescent emission maximum shifted to 480 nm. This is accompanied by a strong enhancement of the fluorescence.

The ThT fluorescence emission spectrum has been confirmed to be consistent with reported data. ThT incubated with α-synuclein fibrils prepared as described in Example 33 has a maximum fluorescence emission wavelength (λₑₘ) of 485 nm and an excitation wavelength (λₑₓ) of 440 nm. No increase in fluorescence emission is observed when ThT is incubated in the presence of monomeric α-synuclein or in α-synuclein free buffer. Furthermore, the ratio of ThT's fluorescence intensity in the presence of α-synuclein fibrils compared to ThT's fluorescence intensity in either monomeric α-synuclein or α-synuclein free buffer has been observed to be about 30-fold.

Accordingly, the α-synuclein fibril binding affinity for a compound may be determined by a method comprising: preparing a plurality of test mixtures comprising α-synuclein fibrils, ThT and a test compound, wherein the test mixtures contain varied concentrations of the test compound; incubating the test mixtures; measuring a fluorescence intensity of each test mixture at the maximum fluorescence emission wavelength and excitation wavelength of ThT; and determining the amount of ThT inhibited from binding to α-synuclein fibrils for each test mixture. The α-synuclein fibril binding affinity for the test compound can then be assessed. The method may also further comprise the steps of preparing a control mixture comprising α-synuclein fibrils and ThT; incubating the control mixture; and measuring a fluorescence intensity of the control mixture at the maximum fluorescence emission wavelength and excitation wavelength of ThT; and determining a ThT-α-synuclein saturation binding curve and dissociation constant (K_{d}).

In another aspect, the present invention is directed to competitive binding assays for the development of a radiotracer for imaging α-synuclein aggregation in vivo. For example, as demonstrated Example 36, α-synuclein fibrils have a binding site for compound SIL23, which is a feasible radiotracer target.

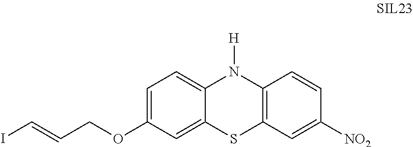 [SMILES: O=[N+]([O-])c1ccc2c(c1)Sc1cc(OC/C=C/I)ccc1N2]

A competitive binding assay utilizing, for example compound [¹²⁵I]SIL23, can be performed to screen additional phenothiazine, phenoxazine, and phenazine analogues as well as other classes of compounds to identify candidate imaging ligands with high affinity and selectivity for α-synuclein.

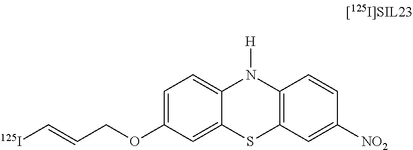 [SMILES: O=[N+]([O-])c1ccc2c(c1)Sc1cc(OC/C=C/[125I])ccc1N2]

SIL23 binding sites are present in a transgenic mouse model over-expressing A53T α-synuclein, indicating that brain uptake and in vivo binding of candidate α-synuclein imaging ligands can be evaluated in this mouse model with micro-PET or ex vivo autoradiography studies.

Accordingly, method for determining the binding affinity of a compound to α-synuclein fibrils in accordance with the present invention comprises: preparing a plurality of test mixtures comprising α-synuclein fibrils, an α-synuclein radioligand compound (e.g., [¹²⁵I]SIL23) and a test compound, wherein the test mixtures contain varied concentrations of the test compound; incubating the test mixtures; measuring the radioactivity of bound and/or unbound radioligand; and determining the amount of radioligand inhibited from binding to α-synuclein fibrils for each test mixture.

### EXAMPLES

The following non-limiting examples are provided to further illustrate the present invention.

Unless otherwise stated, reagents and chemicals were purchased from Sigma-Aldrich Corporation (Milwaukee, Wis.) or VWR International, Inc. (Earth city, MO) and used without further purification unless otherwise stated. The air and water sensitive reactions were carried out under nitrogen. The melting points of all the intermediates and final compounds were determined on a Hake-Buchler melting point apparatus and are uncorrected. ¹H NMR spectra were obtained on a Varian-300 MHz NMR spectrometer. Spectra are referenced to the deuterium lock frequency of the spectrometer. The following abbreviations were used to describe peak patterns when appropriate: br s=broad singlet, s=singlet, d=doublet, t=triplet, q=quartet, m=multiplet. The purity of the target compounds was found to be >95%, as determined by elemental analysis or HPLC.

Plate reader & software used for fluorescence scans was TECAN infinite M100 Plate Reader, and i-control 1.7 TECAN software was used to run plate reader. Plate reader & software used for the binding assay was Biotek Synergy 2 Plate Reader, and Gen 5 software was used to run plate reader. Fluorescence filters were Excitation 440/30, Emission 485/20. Optical Setting Top 50% and Sensitivity=60. UV/Vis absorbance scans were taken on a Beckman Coulter DU 800 spectrophotometer using quartz cuvettes.

### Example 1

### Synthesis of 3,7-dimethoxy-10H-phenothiazine (Compound 6)

3,7-dimethoxy-10H-phenothiazine was prepared according to the following reaction scheme:

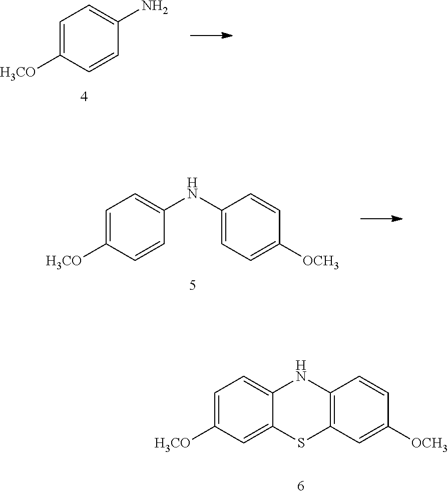 [SMILES: COc1ccc(N)cc1 ; COc1ccc(Nc2ccc(OC)cc2)cc1 ; COc1ccc2c(c1)Sc1cc(OC)ccc1N2]

4-Aminoanisole (0.3 g, 2.5 mmol; Compound 4), 4-bromoanisole (0.36 g, 2 mmol), CuI (75 mg, 0.4 mmol), L-proline (95 mg, 0.8 mmol) and K₂CO₃ (1.1 g, 8 mmol) were mixed in 10 ml of dimethyl sulfoxide (DMSO) and heated at 100° C. for 2 days. The reaction mixture was quenched with water (50 mL) and extracted with ethyl acetate. The organic phase was dried over anhydrous Na₂SO₄ and concentrated. The crude product was purified via column chromatography on silica gel to yield bis(4-methoxy)amine (Compound 5) as a white solid (0.15 g, 33%). ¹H NMR (CDCl₃): δ=3.76 (s, 6H), 5.29 (b, 1H), 6.81 (d, 4H, J=9.0 Hz), 6.93 (d, 4H, J=9.0 Hz); mp 92.4-95.0° C.

Compound 5 (0.15 g, 1.14 mmol), sulfur (91 mg, 2.3 mmol) and iodine (29 mg, 0.1 mmol) were added to 1,2-dichlorobenzene (10 mL). The reaction mixture was heated at 150° C. for 12 hours. The reaction mixture was purified via column chromatography on silica gel to give 50 mg of 3,7-dimethoxy-10H-phenothiazine (Compound 6) as a yellow solid (0.19 mmol, 17%). ¹H NMR (CDCl₃): δ=3.64 (s, 6H), 6.57-6.61 (m, 6H), 8.14 (b, 1H). Anal. calcd for C₁₄H₁₃NO₂S: C, 64.84; H, 5.05; N, 5.40. Found: C, 64.57; H, 5.15; N, 5.21; mp 194.2-196.1° C.

### Example 2

### Synthesis of 7-methoxy-10H-phenothiazine-3-carbonitrile (Compound 11a)

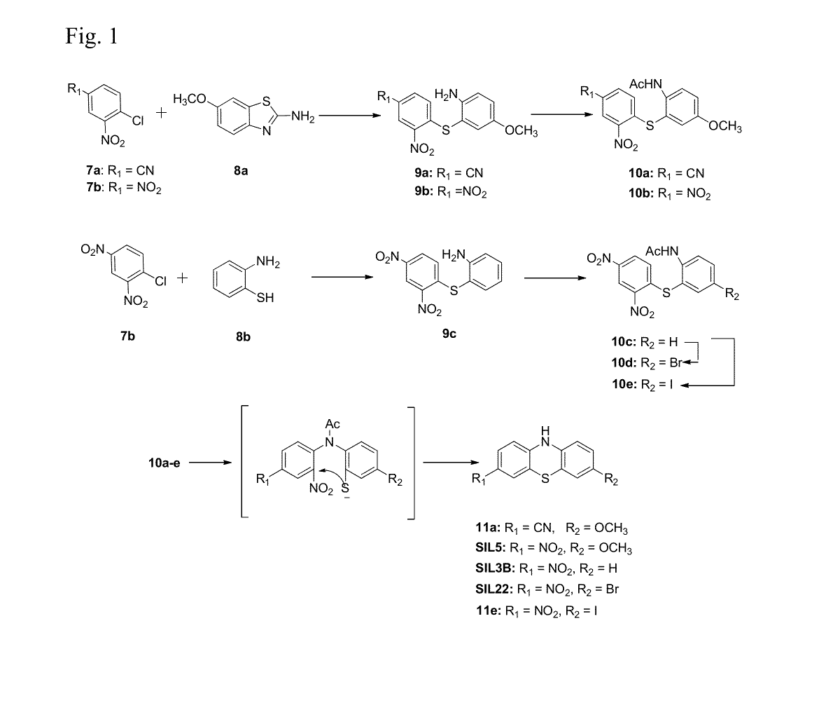

Compound 11a was prepared according to the reaction scheme provided in FIG. 1.

6-Methoxybenzothiazol-2-amine (0.5 g, 2.8 mmol; Compound 8a) was suspended in aqueous potassium hydroxide solution and refluxed overnight. The reaction solution was cooled to room temperature and then added dropwise to a solution of 4-chloro-3-nitrobenzonitrile (0.51 g, 2.8 mmol; Compound 7a) in ethanol (20 mL)/acetic acid (50 mL) in a water bath. The reaction mixture was stirred for an additional 3 hours. The precipitate was filtered and washed with a 1:1 mixture of water:ethanol to give 4-((2-Amino-5-methoxyphenyl)thio)-3-nitrobenzonitrile (Compound 9a) as a red solid. (0.51 g, 60%). ¹H NMR (CDCl₃): δ=3.77 (s, 3H), 3.99 (b, 2H), 6.85 (d, 1H, J=8.4 Hz), 6.98 (m, 3H), 7.58 (d, 1H, J=8.7 Hz), 8.56 (s, 1H); mp 163.6-165.1° C.

Acetic anhydride (10 mL) and pyridine (2 mL) was added to a flask containing Compound 9a (0.3 g, 0.93 mmol). The solution was stirred for about 3 hours at ambient temperature and then quenched with ice-cold water. The precipitate was filtered and then extracted by ethyl acetate from water. The combined organic layer was washed with water and brine, then concentrated in vacuo to give an oil. The crude oil was purified by recrystallization in acetone/water to obtain N-(2-((4-cyano-2-nitrophenyl)thio)-4-methoxy-phenyl)acetamide (Compound 10a) as a yellow solid (0.46 g, 81%). ¹H NMR (CDCl₃): δ=2.91 (s, 3H), 3.82 (s, 3H), 6.89 (d, 1H, J=8.4 Hz), 7.08 (s, 1H), 7.15 (d, 1H, J=9.3 Hz), 7.60 (d, 1H, J=8.7 Hz), 7.66 (b, 1H), 8.32 (d, 1H, J=9.0 Hz), 8.58 (s, 1H); mp 195.0-198.9° C.

Potassium hydroxide (98 mg, 1.74 mmol) was added in portions to a solution of Compound 10a (300 mg, 0.87 mmol) in acetone under reflux. The reaction mixture was heated at reflux for 2 hours and then quenched with ice-cold water. The precipitate was filtrated and then recrystallized in acetone/water to give 7-methoxy-10H-phenothiazine-3-carbonitrile (Compound 11a) as a yellow solid (100 mg, 45%). ¹H NMR (DMSO-d₆): δ=3.67 (s, 3H), 6.62 (m, 4H), 7.34 (m, 2H), 9.02 (b, 1H). Anal. calcd for C₄H₁₀N₂OS: C, 66.12; H, 3.96; N, 11.02. Found: C, 66.13; H, 3.86; N, 11.03; mp 198.0-198.9° C.

### Example 3

### Synthesis of 3-methoxy-7-nitro-10H-phenothiazine (Compound SIL5)

Compound SIL5 was prepared according to the reaction scheme provided in FIG. 1.

Compound 8a (5 g, 28 mmol) was suspended in aqueous potassium hydroxide solution and refluxed overnight. The reaction solution was cooled to room temperature and then added dropwise to a solution of 2,4-dinitrochlorobenzene (6.7 g, 28 mmol; Compound 7b) in ethanol (20 mL)/acetic acid (50 mL) in a water bath. The reaction mixture was stirred for an additional 3 hours. The precipitate was filtered and washed with a 1:1 mixture of water:ethanol to give 2-((2,4-dinitrophenyl)thio)-5-methoxyaniline (Compound 9b) as a yellow solid (7.3 g, 81%). ¹H NMR (CDCl₃): δ=3.76 (s, 3H), 4.00 (b, 2H), 6.85 (d, 1H, J=8.4 Hz), 6.96-7.06 (m, 3H), 8.18 (d, 1H, J=9.0 Hz), 9.13 (s, 1H); mp 169.4-171.7° C.

Acetic anhydride (10 mL) and pyridine (2 mL) was added to a flask containing Compound 9b (0.3 g, 0.93 mmol). The solution was stirred for 3 hours at ambient temperature and then quenched with ice-cold water. The precipitate was filtered and then extracted by ethyl acetate from water. The combined organic layer was washed with water and brine, then concentrated in vacuo to give an oil. The crude oil was purified by recrystallization in acetone/water to afford N-(2-((2,4-dinitrophenyl)thio)-4-methoxyphenyl)acetamide (Compound 10b) as a yellow solid (0.31 g, 95%), ¹H NMR (CDCl₃): δ 2.05 (s, 3H), 3.81 (s, 3H), 6.95 (d, J=9.0 Hz, 1H), 7.09 (s, 1H), 7.16 (d, J=9.0 Hz, 1H), 7.65 (br s, 1H), 8.19 (d, J=9.0 Hz, 1H), 8.33 (d, J=8.7 Hz, 1H), 9.14 (s, 1H). mp 124.6-125.6° C.

Potassium hydroxide (98 mg, 1.74 mmol) was added in portions to a solution of Compound 10b (100 mg, 0.82 mmol) in acetone under reflux. The reaction mixture was heated at reflux for 2 hours and then quenched with ice-cold water. The precipitate was filtrated and then recrystallized in acetone/water to give 3-methoxy-7-nitro-10H-phenothiazine (Compound SIL5) as a violet solid (61 mg, 79%). ¹H NMR (DMSO-d₆): δ=3.66 (s, 3H), 6.58-6.64 (m, 4H), 7.71 (s, 1H), 7.83 (d, 1H, J=9.0 Hz), 9.39 (b, 1H). Anal. calcd for C₁₃H₁₀N₂O₃S: C, 56.92; H, 3.67; N, 10.21. Found: C, 56.64; H, 3.54; N, 10.07; mp 168.8-170.1° C.

### Example 4

### Synthesis of 3-nitro-10H-phenothiazine (Compound SIL3B)

Compound SIL3B was prepared according to the reaction scheme provided in FIG. 1.

A solution of 2,4-dinitrochlorobenzene (10 g, 49 mmol; Compound 7b) in ethanol was added dropwise to a solution of 2-aminobenzenethiol (6.8 g, 54 mmol) and NaOH (2.16 g, 54 mmol) in ethanol. The reaction mixture was stirred at ambient temperature for 2 hours. The precipitate was filtered and washed with ethanol to obtain 2-((2,4-dinitrophenyl)thio)aniline (Compound 9c) as a yellow solid (11.4 g, 88%). ¹H NMR (CDCl₃): δ=4.28 (b, 2H), 6.87 (m, 2H), 7.03 (d, 1H, J=9.0 Hz), 7.42 (m, 2H), 8.17 (d, 1H, J=9.0 Hz), 9.12 (s, 1H); mp 150.7-151.8° C.

Acetic anhydride (10 mL) and pyridine (2 mL) was added to a flask containing Compound 9c (1.1 g, 3.8 mmol). The solution was stirred for 3 hours at ambient temperature and then quenched with ice-cold water. The precipitate was filtered and then extracted by ethyl acetate from water. The combined organic layer was washed with water and brine, then concentrated in vacuo to give an oil. The crude oil was purified by recrystallization in acetone/water to obtain N-(2-((2,4-dinitrophenyl)thio)phenyl)acetamide (Compound 10c) as a yellow solid (12.5 g, 96%). ¹H NMR (CDCl₃): δ=2.10 (s, 3H), 6.88 (d, 1H, J=8.7 Hz), 7.26 (t, 1H, J=6.3 Hz), 7.59 (m, 2H), 7.94 (b, 1H), 8.19 (d, 1H, J=9.0 Hz), 8.54 (d, 1H, J=8.4 Hz), 9.14 (s, 1H); mp 182.7-184.0° C.

Potassium hydroxide (98 mg, 1.74 mmol) was added in portions to a solution of compound 10c (300 mg, 0.9 mmol) in acetone under reflux. The reaction mixture was heated at reflux for 2 hours and then quenched with ice-cold water. The precipitate was filtrated and then recrystallized in acetone/water to give 3-nitro-10H-phenothiazine (Compound SIL3B) as a violet solid (0.16 g, 73%). ¹H NMR (DMSO-d₆): δ=6.69 (m, 2H), 6.84 (t, 1H, J=7.2 Hz), 6.93 (d, 1H, J=7.2 Hz), 7.02 (t, 1H, J=7.2 Hz), 7.73 (s, 1H), 7.85 (d, 1H, J=8.7 Hz), 9.51 (b, 1H). Anal. calcd for C₁₂H₈N₂O₂S: C, 59.00; H, 3.30; N, 11.47. Found: C, 59.02; H, 3.26; N, 11.33; mp 219.1-219.6° C.

### Example 5

### Synthesis of 3-bromo-7-nitro-10H-phenothiazine (Compound SIL22)

Compound SIL22 was prepared according to the reaction scheme provided in FIG. 1.

Compound 9c (2.0 g, 6 mmol) and N-bromosuccinimide (4.0 g, 24 mmol) were dissolved in 5 mL of dimethylformamide (DMF). The reaction mixture was heated at 100° C. overnight and then quenched with 100 mL of water. The precipitate was filtered and purified via column chromatography on silica gel to give N-(5-bromo-2-((2,4-dinitrophenyl)thio)phenyl)acetamide (Compound 10d) as a yellow solid (2.3 g, 90%). ¹H NMR (CDCl₃): δ=2.11 (s, 3H), 6.91 (d, 1H, J=9.0 Hz), 7.73 (m, 2H), 7.91 (b, 1H), 8.25 (d, 1H, J=9.0 Hz), 8.51 (d, 1H, J=9.3 Hz), 9.16 (s, 1H); mp 193.7-195.7° C.

Potassium hydroxide (98 mg, 1.74 mmol) was added in portions to a solution of compound 10d (240 mg, 0.9 mmol) in acetone under reflux. The reaction mixture was heated at reflux for 2 hours and then quenched with ice-cold water. The precipitate was filtrated and then recrystallized in acetone/water to give 3-bromo-7-nitro-10H-phenothiazine (Compound SIL22) as a violet solid (87 mg, 45%). ¹H NMR (DMSO-d₆): δ=6.60 (d, 1H, J=8.4 Hz), 6.67 (d, 1H, J=8.4 Hz), 7.17 (m, 2H), 7.73 (s, 1H), 7.85 (d, 1H, J=9.0 Hz), 9.59 (s, 1H). Anal. calcd for C₁₂H₇BrN₂O₂S: C, 44.60; H, 2.18; N, 8.67. Found: C, 44.54; H, 2.26; N, 8.56; mp>250° C.

### Example 6

### Synthesis of 3-iodo-7-nitro-10H-phenothiazine (Compound 11e)

Compound 11e was prepared according to the reaction scheme provided in FIG. 1.

Compound 10c (0.5 g, 1.5 mmol) was dissolved in acetic acid (20 mL). To this solution under nitrogen atmosphere was added iodine monochloride (10 mL, 1.0 M in methylene chloride). The reaction mixture was heated at reflux for 3 days and then quenched with water. After filtration, the residue was purified on silica gel column chromatography to obtain N-(2-((2,4-dinitrophenyl)thio)-5-iodophenyl)acetamide (compound 10e) as a yellow solid (220 mg, 32%). ¹H NMR (CDCl₃): δ=2.08 (s, 3H), 6.90 (d, 1H, J=9.3 Hz), 7.87 (m, 2H), 7.99 (b, 1H), 8.23 (d, 1H, J=8.7 Hz), 8.31 (d, 1H, J=8.4 Hz), 9.11 (s, 1H); mp 224.1-226.0° C.

Potassium hydroxide (98 mg, 1.74 mmol) was added in portions to a solution of compound 10e (100 mg, 0.22 mmol) in acetone under reflux. The reaction mixture was heated at reflux for 2 hours and then quenched with ice-cold water. The precipitate was filtrated and then recrystallized in acetone/water to give 3-iodo-7-nitro-10H-phenothiazine (Compound 11e) as a violet solid (0.29 g, 36%). ¹H NMR (DMSO-d₆): δ=6.45 (d, 1H, J=8.7 Hz), 6.64 (d, 1H, J=9.3 Hz), 7.22 (s, 1H), 7.30 (d, 1H, J=9.3 Hz), 7.69 (s, 1H), 7.82 (d, 1H, J=8.7 Hz), 9.54 (b, 1H). HRMS (ESI, m/z) calcd for C₁₂H₇₁N₂O₂S [M⁺] 369.9276. Found: 369.9269. HPLC purity 97%; mp>250° C.

### Example 7

### Synthesis of 3-methoxy-10-methyl-7-nitro-10H-phenothiazine (Compound 12)

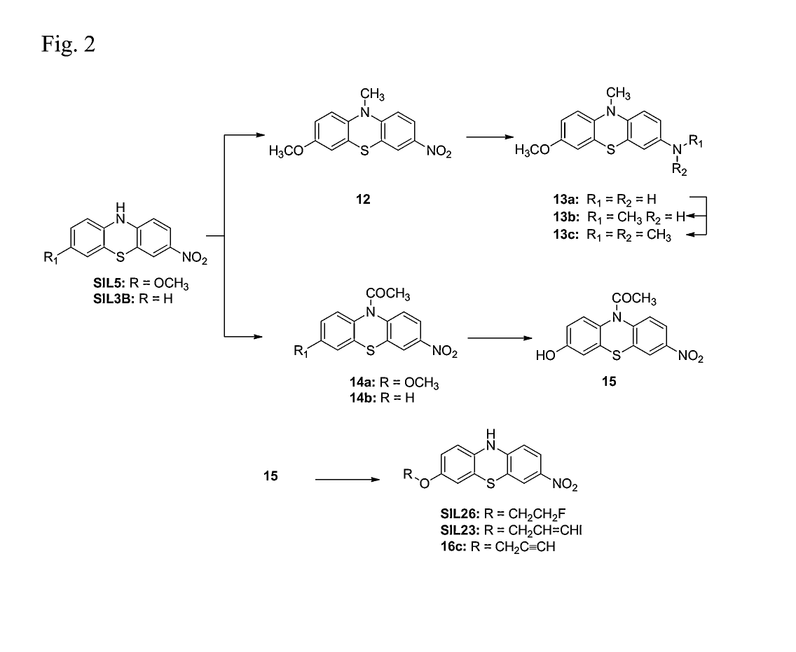

Compound 12 was prepared according to the reaction scheme provided in FIG. 2.

Sodium hydride (29 mg, 0.73 mmol) was added to a solution of Compound SIL5 (100 mg, 0.36 mmol) in 10 mL of DMF e at 0° C. The reaction mixture was stirred for 30 minutes and then warmed to room temperature. Methyl iodide (103 mg, 0.73 mmol) was added to the solution and then the reaction mixture was stirred for an additional 2 hours. The reaction mixture was quenched with water and then extracted with ethyl acetate. The combined organic extracts were dried and concentrated. The residue was purified via column chromatography on silica gel to obtain 3-methoxy-10-methyl-7-nitro-10H-phenothiazine (Compound 12) as a red solid (90 mg, 87%). ¹H NMR (DMSO-d₆): δ=3.28 (s, 3H), 3.63 (s, 3H), 6.71-6.76 (m, 2H), 6.87-6.94 (m, 2H), 7.84 (s, 1H), 7.96 (d, 1H, J=9.3 Hz). Anal. calcd for C₁₄H₁₂N₂O₃S: C, 58.32; H, 4.20; N, 9.72. Found: C, 58.11; H, 4.11; N, 9.75; mp 174.7-175.5° C.

### Example 8

### Synthesis of 7-methoxy-10-methyl-10H-phenothiazin-3-amine (Compound 13a)

Compound 13a was prepared according to the reaction scheme provided in FIG. 2.

Compound 12 (0.5 g, 1.7 mmol) and palladium on carbon (20 mg) were suspended in ethanol (15 mL). The reaction mixture was stirred at ambient temperature under (1 atm) of hydrogen overnight, then filtered and concentrated in vacuo. The crude product was recrystallized in dichloromethane/hexane to give 7-methoxy-10-methyl-10H-phenothiazin-3-amine as a yellowish solid (0.35 g, 80%). ¹H NMR (DMSO-d₆): δ=3.15 (s, 3H), 3.69 (s, 3H), 4.79 (b, 2H), 6.42-6.45 (m, 2H), 6.64 (d, 1H, J=9.3 Hz), 6.73-6.80 (m, 3H). Anal. calcd for C₁₄H₁₄N₂OS: C, 65.09; H, 5.46; N, 10.84. Found: C, 65.29; H, 5.53; N, 10.59; mp 141.6-142.9° C.

### Example 9

### Synthesis of 7-methoxy-N,10-dimethyl-10H-phenothiazin-3-amine (Compound 13b) and 7-methoxy-N,N,10-trimethyl-10H-phenothiazin-3-amine (Compound 13c)

Compounds 13b and 13c were prepared according to the reaction scheme provided in FIG. 2.

Compound 13a (200 mg, 0.77 mmol), methyl iodide (0.28 g, 2 mmol) and sodium carbonate (0.21 g, 2 mmol) were mixed in 10 mL of acetonitrile, then stirred at 80° C. overnight. After cooling to room temperature, the reaction mixture was partitioned between ethyl acetate and water. The organic extract was purified via column chromatography to give 7-methoxy-N,10-dimethyl-10H-phenothiazin-3-amine (Compound 13b) (32 mg, 15%) and 7-methoxy-N,N,10-trimethyl-10H-phenothiazin-3-amine (Compound 13c) (57 mg, 26%) as yellow solids. Compound 13b: ¹H NMR (DMSO-d₆): δ=2.58 (s, 3H), 3.66 (s, 3H), 3.73 (m, 4H), 6.39 (m, 2H), 6.74 (m, 4H). Anal. calcd for C₁₅H₁₆N₂OS: C, 66.15; H, 5.92; N, 10.29. Found: C, 66.36; H, 6.09; N, 10.24; mp 170.9-171.7° C. Compound 13c: ¹H NMR (DMSO-d₆): δ=2.80 (s, 6H), 3.70 (s, 3H), 3.76 (m, 3H), 6.62 (m, 2H), 6.80 (m, 4H). Anal. calcd for C₁₆H₁₈N₂OS: C, 67.10; H, 6.33; N, 9.78. Found: C, 67.36; H, 6.25; N, 9.79; mp 185.0-186.5° C.

### Example 10

### Synthesis of 1-(3-methoxy-7-nitro-10H-phenothiazin-10-yl)ethanone (Compound 14a)

Compound 14a was prepared according to the reaction scheme provided in FIG. 2.

Acetyl chloride (0.85 g, 11 mmol) was added to a solution of compound SIL5 (1 g, 3.6 mmol) in dichloromethane (20 mL). The reaction mixture was allowed to stir overnight at room temperature, then the solvent and unreacted acetyl chloride were removed in vacuo. The residue was dissolved in ethyl acetate and washed with water and brine. The organic layer was then dried over anhydrous sodium sulfate and purified via column chromatography on silica gel to give 1-(3-methoxy-7-nitro-10H-phenothiazin-10-yl)ethanone (Compound 14a) as a yellow solid (0.4 g, 89%). ¹H NMR (CDCl₃): δ=2.23 (s, 3H), 3.83 (s, 3H), 6.90 (d, 1H, J=9.0 Hz), 6.98 (s, 1H), 7.32 (d, 1H, J=8.7 Hz), 7.72 (d, 1H, J=8.7 Hz), 8.18 (d, 1H, J=8.7 Hz), 8.29 (s, 1H). Anal. calcd for C₁₅H₁₂N₂O₄S: C, 56.95; H, 3.82; N, 8.86. Found: C, 56.72; H, 3.89; N, 8.70; mp 155.9-156.8° C.

### Example 11

### Synthesis of 1-(3-nitro-10H-phenothiazin-10-yl)ethanone (Compound 14b)

Compound 14b was prepared according to the reaction scheme provided in FIG. 2.

Acetyl chloride (0.85 g, 11 mmol) was added to a solution of compound SIL3B (150 mg, 0.6 mmol) in dichloromethane (20 mL). The reaction mixture was allowed to stir overnight at room temperature, and then the solvent and unreacted acetyl chloride were removed in vacuo. The residue was dissolved in ethyl acetate and washed with water and brine. The organic layer was then dried over anhydrous sodium sulfate and purified via column chromatography on silica gel to give 1-(3-nitro-10H-phenothiazin-10-yl)ethanone (Compound 14b) as a yellow solid (110 g, 62%). ¹H NMR (DMSO-d₆): δ=2.18 (s, 3H), 7.36 (t, 1H, J=7.2 Hz), 7.45 (t, 1H, J=7.2 Hz), 7.61 (d, 1H, J=7.8 Hz), 7.69 (d, 1H, J=7.8 Hz), 7.86 (d, 1H, J=7.8 Hz), 8.24 (d, 1H, J=8.4 Hz), 8.42 (s, 1H). Anal. calcd for C₁₄H₁₀N₂O₃S: C, 58.73; H, 3.52; N, 9.78. Found: C, 58.60; H, 3.61; N, 9.62; mp 144.0-145.8° C.

### Example 12

### Synthesis of 1-(3-hydroxy-7-nitro-10H-phenothiazin-10-yl)ethanone (Compound 15)

Compound 15 was prepared according to the reaction scheme provided in FIG. 2.

A solution of boron tribromide in dichloromethane (1 mL) was added dropwise to a solution of compound 14a (100 mg, 0.32 mmol) in dichloromethane (10 mL) at −78° C. The reaction solution was stirred overnight at room temperature, then the solvent was removed in vacuo and the residue partitioned between ethyl acetate and water. The organic layer was dried over anhydrous sodium sulfate and purified via column chromatography on silica to give 1-(3-hydroxy-7-nitro-10H-phenothiazin-10-yl)ethanone (Compound 15) as a yellowish solid (79 mg, 81%). ¹H NMR (DMSO-d₆): δ=2.15 (s, 3H), 6.82 (d, 1H, J=9.0 Hz), 6.93 (s, 1H), 7.47 (d, 1H, J=9.0 Hz), 7.82 (d, 1H, J=9.0 Hz), 8.22 (d, 1H, J=9.0 Hz), 8.39 (s, 1H), 10.00 (b, 1H). FIRMS (ESI, m/z) [M+1] calcd for C₁₄H₁₀N₂O₄S: 303.0440. Found: 303.0435. HPLC purity 98%; mp 202.3-205.1° C.

### Example 13

### Synthesis of 3-(2-fluoroethoxy)-7-nitro-10H-phenothiazine (Compound SIL26)

Compound SIL26 was prepared according to the reaction scheme provided in FIG. 2.

Compound 15 (120 mg, 0.4 mmol) was dissolved in 10 mL of anhydrous dimethylformamide, then to this solution at 0° C. was added sodium hydride (24 mg, 0.6 mmol). The reaction mixture was stirred for 30 minutes and then to it added 1-bromo-2-fluoroethane (150 mg, 0.6 mmol). The reaction mixture was stirred at room temperature overnight, then quenched with water (100 mL) and extracted with ethyl acetate. After the removal of solvent, the residue was suspended in a 1:1 mixture of 3M aqueous hydrochloric acid and methanol and heated at reflux for 5 hours. The reaction mixture was then quenched with water and extracted with ethyl acetate. The organic layer was dried over anhydrous sodium sulfate and purified via column chromatography on silica gel to give 3-(2-fluoroethoxy)-7-nitro-10H-phenothiazine (Compound SIL26) as a violet solid (40 mg, 33%). ¹H NMR (DMSO-d₆): δ=4.09 (t, 1H, J=3.6 Hz), 4.19 (t, 1H, J=3.6 Hz), 4.60 (t, 1H, J=3.6 Hz), 4.76 (t, 1H, J=3.6 Hz), 6.64 (m, 4H), 7.72 (s, 1H), 7.83 (d, 1H, J=9.3 Hz), 9.41 (s, 1H). Anal. calcd for C15H10N2O3S: C, 60.39; H, 3.38; N, 9.39. Found: C, 60.15; H, 3.50;N, 9.15; mp 189.3-191.4° C.

### Example 14

### Synthesis of (E)-3-(3-iodoallyloxy)-7-nitro-10H-phenothiazine (Compound SIL23)

Compound SIL23 was prepared according to the reaction scheme provided in FIG. 2.

Prop-2-yn-1-ol (0.7 g, 12 mmol), bis(triphenylphosphine)palladium(II) dichloride (42 mg, 0.06 mmol) and tributyltin hydride (3 g, 10 mmol) were dissolved in anhydrous tetrahydrofuran and stirred at room temperature for 1 hour. The solvent was then removed in vacuo and the residue purified via column chromatography on silica gel to give 1.2 g of (E)-3-(tributylstannyl)prop-2-en-1-ol (34%) as a colorless liquid.

A 0.1M solution of iodine in chloroform (10 mL) was added to a solution of (E)-3-(tributylstannyl)prop-2-en-1-ol (200 mg, 0.58 mmol) in 10 mL of chloroform and stirred at room temperature for 2 hours. The reaction mixture was quenched with 5% aqueous sodium metabisulfite (Na₂S₂O₅) and extracted with ethyl acetate. The organic extract was concentrated and purified via column chromatography on silica gel column to give ((E)-3-iodoprop-2-en-1-ol) as a colorless liquid (85 mg, 79%). ¹H NMR (CDCl₃): δ=2.04 (t, 1H, J=5.7 Hz), 4.08 (t, 2H, J=5.7 Hz), 6.39 (d, 1H, J=14.4 Hz), 6.68 (d, 1H, J=14.4 Hz).

Triphenylphosphine (133 mg, 0.51 mmol) was added to a solution of (E)-3-iodoprop-2-en-1-ol in 10 mL of methylene chloride at 0° C. The reaction mixture was stirred at 0° C. for 1 hour, then to it added carbon tetrabromide (186 mg, 0.56 mmol). After stirring for an additional 2 hours at room temperature, the solvent was removed in vacuo. The residue was purified via column chromatography on silica gel to give (E)-3-bromo-1-iodoprop-1-ene as a colorless liquid (40 mg, 35%). ¹H NMR (CDCl₃): δ=3.87 (d, 2H, J=7.8 Hz), 6.54 (d, 1H, J=14.4 Hz), 6.71 (d, 1H, J=14.4 Hz).

Compound 15 (50 mg, 0.17 mmol), (E)-3-bromo-1-iodoprop-1-ene (50 mg, 0.2 mmol) and sodium carbonate (200 mg, 0.2 mmol) were mixed in dimethylformamide and stirred for 3 hours at room temperature. The reaction mixture was quenched with water, extracted with ethyl acetate, and the organic layer concentrated in vacuo. The residue was suspended in a 1:1 mixture of 3 M aqueous hydrochloric acid and methanol and then heated at reflux for 6 hours. The precipitate was filtered to give (E)-3-(3-iodoallyloxy)-7-nitro-10H-phenothiazine (Compound SIL23) as a violet solid (25 mg, 36%). ¹H NMR (DMSO-d₆): δ=4.41 (s, 2H), 6.61-6.71 (m, 6H), 7.71 (s, 1H), 7.83 (d, 1H, J=9.0 Hz), 9.41 (b, 1H). Anal. calcd for C15H11IN2O35: C, 42.27; H, 2.60; N, 6.57. Found: C, 42.46; H, 2.72; N, 6.38. mp>250° C.

### Example 15

### Synthesis of 3-nitro-7-(prop-2-yn-1-yloxy)-10H-phenothiazine (Compound 16c)

Compound 16c was prepared according to the reaction scheme provided in FIG. 2.

Compound 15 (50 mg, 0.17 mmol), 3-bromopropyne (38 mg, 0.25 mmol) and sodium carbonate (200 mg, 0.2 mmol) were mixed in dimethylformamide and stirred for 3 hours at room temperature. The reaction mixture was quenched with water, extracted with ethyl acetate, and the organic layer concentrated in vacuo. The residue was suspended in a 1:1 mixture of 3 M aqueous hydrochloric acid and methanol and then heated at reflux for 6 hours. The precipitate was filtered to give 3-nitro-7-(prop-2-yn-1-yloxy)-10H-phenothiazine (Compound 16c) as a violet solid (55 mg, 86%). ¹H NMR (DMSO-d₆): δ=3.58 (s, 1H), 4.71 (s, 2H), 6.62-6.67 (s, 4H), 7.73 (s, 1H), 7.84 (d, 1H, J=9.0 Hz), 9.43 (b, 1H). Anal. calcd for C₁₄H₁₀N₂O₃S: C, 54.89; H, 3.62; N, 9.15. Found: C, 54.87; H, 3.54; N, 8.92; mp 226.4-227.0° C.

### Example 16

### Synthesis of 3-methoxy-7-nitro-10H-phenoxazine (Compound TZ-2-33)

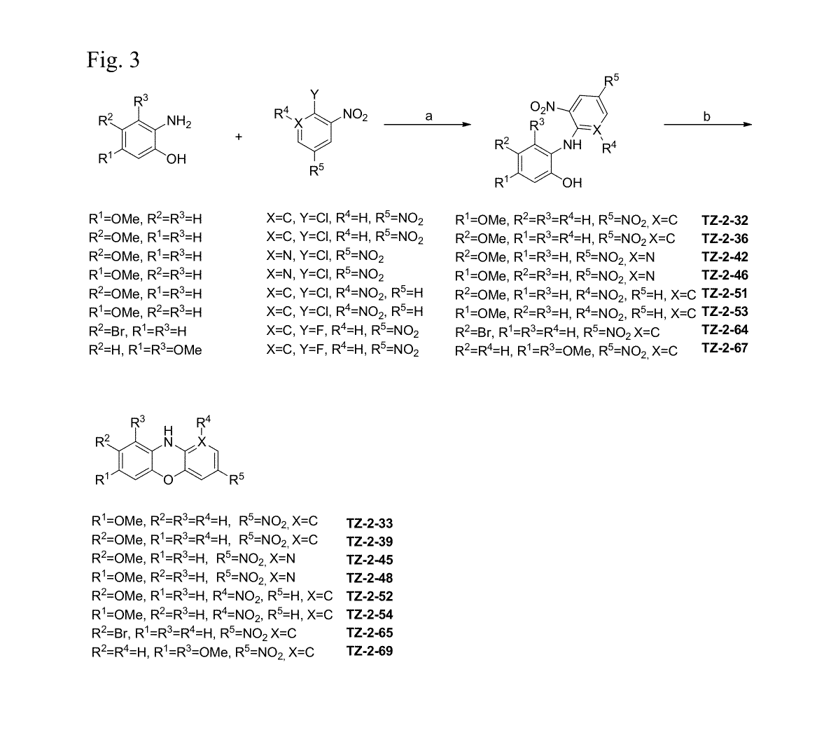

Compound TZ-2-33 was synthesized according to the reaction scheme provided in FIG. 3.

In the first step, 2-amino-5-methoxyphenol hydrochloride (100 mg, 0.57 mmol), 1-chloro-2,4-dinitrobenzene (122 mg, 0.6 mmol), and sodium acetate (164 mg, 2 mmol) were dissolved in 5 mL of ethanol and 1 mL of water. The reaction mixture was heated at reflux for 24 hours and then cooled to room temperature. The precipitate was filtered and washed with ethanol and water to give 2-[(2,4-dinitrophenyl)amino]-5-methoxyphenol (Compound TZ-2-32) as a reddish solid (143 mg, 82%). ¹H NMR (DMSO-d₆): δ 3.75 (s, 3H), 6.52 (d, 1H, J=8.7 Hz), 6.57 (s, 1H), 6.77 (d, 1H, J=9.6 Hz), 7.17 (d, 1H, J=9.0 Hz), 8.21 (d, 1H, J=9.6 Hz), 8.89 (s, 1H), 9.81 (b, 1H), 9.96 (b, 1H).

In the second step, TZ-2-32 (50 mg, 0.16 mmol) and potassium carbonate (44 mg, 0.32 mmol) were mixed in 5 mL of dimethylformamide and then heated at 120° C. for 5 hours. The reaction was quenched with cold water (10 mL) and extracted with ethyl acetate (3×10 mL), the combined organic layer was washed with water and concentrated under reduced pressure. After purification by column chromatography on silica gel, 3-methoxy-7-nitro-10H-phenoxazine (Compound TZ-2-33) was obtained as a red solid (19 mg, 46%): mp 215-216° C.; ¹H NMR (DMSO-d₆): δ 3.65 (s, 3H), 6.33-6.47 (m, 4H), 7.28 (s, 1H), 7.66 (d, 1H, J=9.0 Hz), 9.24 (b, 1H).

### Example 17

### Synthesis of 2-methoxy-7-nitro-10H-phenoxazine (Compound TZ-2-39)

Compound TZ-2-39 was synthesized according to the reaction scheme provided in FIG. 3.

In the first step, 2-amino-4-methoxyphenol hydrochloride (300 mg, 2.15 mmol), 1-chloro-2,4-dinitrobenzene (480 mg, 2.37 mmol), and sodium acetate (820 mg, 10 mmol) were dissolved in 5 mL of ethanol and 1 mL of water. The reaction mixture was heated at reflux for 24 hours and then cooled to room temperature. The precipitate was filtered and washed with ethanol and water to give 2-[(2,4-dinitrophenyl)amino]-4-methoxyphenol (Compound TZ-2-36) as a reddish solid (469 mg, 71%). ¹H NMR (DMSO-d₆): δ 3.70 (s, 3H), 6.83 (d, 1H, J=9.0 Hz), 6.90-6.96 (m, 3H), 8.24 (d, 1H, J=9.6 Hz), 8.90 (s, 1H), 9.50 (b, 1H), 9.91 (b, 1H).

In the second step, TZ-2-36 (200 mg, 0.65 mmol) and potassium carbonate (180 mg, 1.3 mmol) were mixed in 5 mL of dimethylformamide and then heated at 120° C. for 5 hours. The reaction was quenched with cold water (10 mL) and extracted with ethyl acetate (3×10 mL), the combined organic layer was washed with water and concentrated under reduced pressure. After purification by column chromatography on silica gel, 2-methoxy-7-nitro-10H-phenoxazine (Compound TZ-2-39) was obtained as a red solid (112 mg, 67%): mp 220-221° C.; ¹H NMR (DMSO-d₆): δ 3.63 (s, 3H), 6.07 (s, 1H), 6.21 (d, 1H, J=9.0 Hz), 6.46 (d, 1H, J=8.7 Hz), 6.56 (d, 1H, J=8.4 Hz), 7.24 (s, 1H), 7.63 (d, 1H, J=9.0 Hz), 9.31 (b, 1H).

### Example 18

### Synthesis of 8-methoxy-3-nitro-10H-benzo[b]pyrido[2,3-e][1,4]oxazine (Compound TZ-2-45)

Compound TZ-2-45 was synthesized according to the reaction scheme provided in FIG. 3.

In the first step, 2-amino-4-methoxyphenol hydrochloride (200 mg, 1.43 mmol), 1-chloro-2,4-dinitropyridine (322 mg, 1.58 mmol), and sodium acetate (410 mg, 5 mmol) were dissolved in 5 mL of ethanol and 1 mL of water. The reaction mixture was heated at reflux for 24 hours and then cooled to room temperature. The precipitate was filtered and washed with ethanol and water to give 2-[(3,5-dinitropyridin-2-yl)amino]-4-methoxyphenol (Compound TZ-2-42) as a reddish solid (350 mg, 80%). ¹H NMR (DMSO-d₆): δ 3.71 (s, 3H), 6.68 (d, 1H, J=7.2 Hz), 6.89 (d, 1H, J=9.3 Hz), 8.03 (s, 1H), 9.07 (s, 1H), 9.38 (s, 1H), 10.01 (b, 1H), 10.97 (b, 1H).

In the second step, TZ-2-42 (306 mg, 1 mmol) and potassium carbonate (276 mg, 2 mmol) were mixed in 5 mL of dimethylformamide and then heated at 120° C. for 5 hours. The reaction was quenched with cold water (10 mL) and extracted with ethyl acetate (3×10 mL), the combined organic layer was washed with water and concentrated under reduced pressure. After purification by column chromatography on silica gel, 8-methoxy-3-nitro-10H-benzo[b]pyrido[2,3-e][1,4]oxazine (Compound TZ-2-45) was obtained as a red solid (180 mg, 70%): mp 262-263° C.; ¹H NMR (DMSO-d₆): mp 262-263° C.; 6 3.65 (s, 3H), 6.25 (s, 1H), 6.31 (d, 1H, J=9.0 Hz), 6.65 (d, 1H, J=8.4 Hz), 7.42 (s, 1H), 8.45 (s, 1H), 10.29 (b, 1H).

### Example 19

### Synthesis of 7-methoxy-3-nitro-10H-benzo[b]pyrido[2,3-e][1,4]-oxazine (Compound TZ-2-48)

Compound TZ-2-48 was synthesized according to the reaction scheme provided in FIG. 3.

In the first step, 2-amino-5-methoxyphenol hydrochloride (200 mg, 1.13 mmol), 2-chloro-3,5-dinitropyridine (232 mg, 1.13 mmol), and sodium acetate (410 mg, 5 mmol) were dissolved in 5 mL of ethanol and 1 mL of water. The reaction mixture was stirred at room temperature for 4 hours. The precipitate was filtered and washed with ethanol and water, then purified by column chromatography on silica gel to give 2-[(3,5-dinitropyridin-2-yl)amino)-5-methoxyphenol (Compound TZ-2-46) as a red solid (120 mg, 68%). ¹H NMR (DMSO-d₆): δ 3.73 (s, 3H), 6.48 (d, J=9.0 Hz, 1H), 6.54 (s, 1H), 8.02 (d, J=9.0 Hz, 1H), 9.04 (s, 1H), 9.28 (s, 1H), 10.39 (b, 1H), 10.75 (b, 1H).

In the second step, TZ-2-46 (120 mg, 0.39 mmol) and potassium carbonate (106 mg, 0.78 mmol) were mixed in 5 mL of dimethylformamide and heated at 120° C. for 5 hours. The reaction was quenched with cold water (10 mL) and extracted with ethyl acetate (3×10 mL), the combined organic layer was washed with water and concentrated under reduced pressure. After purification by column chromatography on silica gel, 7-methoxy-3-nitro-10H-benzo[b]pyrido[2,3-e][1,4]-oxazine (Compound TZ-2-48) was obtained as a red solid (53 mg, 52%): mp 301-303° C.; ¹H NMR (DMSO-d₆): δ 3.67 (s, 3H), 6.40 (s, 1H), 6.46 (d, J=8.1 Hz, 1H), 6.62 (d, J=8.1 Hz, 1H), 7.42 (s, 1H), 8.46 (s, 1H), 10.29 (b, 1H).

### Example 20

### Synthesis of 8-methoxy-1-nitro-10H-phenoxazine (Compound TZ-2-52)

Compound TZ-2-52 was synthesized according to the reaction scheme provided in FIG. 3.

In the first step, 2-amino-4-methoxyphenol hydrochloride (300 mg, 1.71 mmol), 1-chloro-2,4-dinitrobenzene (346 mg, 1.71 mmol), and sodium acetate (246 mg, 3 mmol) were dissolved in 5 mL of ethanol and 1 mL of water. The reaction mixture was stirred at room temperature for 4 hours. The precipitate was filtered and washed with ethanol and water, then purified by column chromatography on silica gel to give 2-[(2,6-dinitrophenyl)amino]-4-methoxyphenol (Compound TZ-2-51) as a red solid (367 mg, 58%). ¹H NMR (DMSO-d₆): δ 3.59 (s, 3H), 6.43 (s, 1H), 6.49 (d, J=9.0 Hz, 1H), 6.76 (d, J=8.7 Hz, 1H), 7.14 (t, J=8.1 Hz, 1H), 8.32 (d, J=8.7 Hz, 1H), 9.22 (b, 1H), 9.50 (b, 1H).

In the second step, TZ-2-51 (305 mg, 1 mmol) and potassium carbonate (276 mg, 2 mmol) were mixed in 5 mL of dimethylformamide and heated at 120° C. for 5 hours. The reaction was quenched with cold water (10 mL) and extracted with ethyl acetate (3×10 mL), the combined organic layer was washed with water and concentrated under reduced pressure. After purification by column chromatography on silica gel, 8-methoxy-1-nitro-10H-phenoxazine (Compound TZ-2-52) was obtained as a violet solid (210 mg, 81%): mp 179-181° C.; ¹H NMR (DMSO-d₆): δ 3.65 (s, 3H), 6.32 (d, J=8.7 Hz, 1H), 6.63-6.71 (m, 2H), 6.87 (s, 1H), 6.92 (d, J=7.2 Hz, 1H), 7.54 (d, J=8.7 Hz, 1H), 9.32 (b, 1H).

### Example 21

### Synthesis of 7-methoxy-1-nitro-10H-phenoxazine (Compound TZ-2-54)

Compound TZ-2-54 was synthesized according to the reaction scheme provided in FIG. 3.

In the first step, 2-amino-5-methoxyphenol hydrochloride (300 mg, 1.71 mmol), 1-chloro-2,6-dinitrobenzene (346 mg, 1.71 mmol), and sodium acetate (246 mg, 3 mmol) were dissolved in 5 mL of ethanol and 1 mL of water. The reaction mixture was stirred at room temperature for 4 hours. The precipitate was filtered and washed with ethanol and water, then purified by column chromatography on silica gel to give 2-((2,6-dinitrophenyl)amino)-5-methoxyphenol (Compound TZ-2-53) as a red solid (187 mg, 36%). ¹H NMR (DMSO-d₆): δ 3.67 (s, 3H), 6.40 (s, 1H), 6.43 (d, J=9.0 Hz, 1H), 6.63 (t, J=8.1 Hz, 1H), 6.91 (d, J=7.8 Hz, 1H), 7.09 (d, J=8.7 Hz, 1H), 7.54 (d, J=9.0 Hz, 1H), 9.34 (b, 1H).

In the second step, TZ-2-53 (150 mg, 0.5 mmol) and potassium carbonate (138 mg, 1 mmol) were mixed in 5 mL of dimethylformamide and heated at 120° C. for 5 hours. The reaction was quenched with cold water (10 mL) and extracted with ethyl acetate (3×10 mL), the combined organic layer was washed with water and concentrated under reduced pressure. After purification by column chromatography on silica gel, 7-methoxy-1-nitro-10H-phenoxazine (TZ-2-54) was obtained as a violet solid (97 mg, 75%): mp 197-199° C.; ¹H NMR (DMSO-d₆, 300 MHz): δ 3.67 (s, 3H), 6.39 (s, 1H), 6.43 (d, 1H, J=9.0 Hz), 6.63 (t, 1H, J=8.7 Hz), 6.91 (d, 1H, J=7.8 Hz), 7.14 (d, 1H, J=8.7 Hz), 7.54 (d, 1H, J=9.0 Hz), 9.34 (s, 1H).

### Example 22

### Synthesis of 8-bromo-3-nitro-10H-benzo[b]pyrido[2,3-e][1,4]oxazine (Compound TZ-2-65)

Compound TZ-2-65 was synthesized according to the reaction sequence shown in FIG. 3.

In the first step, 2-amino-4-bromophenol hydrochloride (500 mg, 2.7 mmol), 1-fluoro-2,4-dinitrobenzene (500 mg, 2.7 mmol), and sodium acetate (450 mg, 5.4 mmol) were dissolved in 5 mL of ethanol and 1 mL of water. The reaction mixture was stirred at room temperature for 4 hours. The precipitate was filtered and washed with ethanol and water, then purified by column chromatography on silica gel to give 4-bromo-2-((2,4-dinitrophenyl)amino)phenol (Compound TZ-2-64) as a red solid (200 mg, 56%). ¹H NMR (DMSO-d₆): δ 6.87 (d, J=9.6 Hz, 1H), 6.97 (d, J=8.4 Hz, 1H), 7.39 (d, J=8.4 Hz, 1H), 7.49 (s, 1H), 8.25 (d, J=9.3 Hz, 1H), 9.20 (b, 1H), 10.33 (b, 1H).

In the second step, TZ-2-64 (200 mg, 0.56 mmol) and potassium carbonate (276 mg, 2 mmol) were mixed in 5 mL of dimethylformamide and heated at 120° C. for 5 hours. The reaction was quenched with cold water (10 mL) and extracted with ethyl acetate (3×10 mL), the combined organic layer was washed with water and concentrated under reduced pressure. After purification by column chromatography on silica gel, 8-bromo-3-nitro-10H-benzo[b]pyrido[2,3-e][1,4]oxazine (TZ-2-65) was obtained as a violet solid (100 mg, 58%): mp 275-277° C.; ¹H NMR (DMSO-d₆, 300 MHz): δ 3.34 (s, 3H), 6.52 (d, 1H, J=8.4 Hz), 6.59-6.62 (m, 2H), 6.83 (d, 1H, J=8.4 Hz), 7.33 (s, 1H), 7.69 (d, 1H, J=8.4 Hz), 9.45 (s, 1H).

### Example 23

### Synthesis of 7,9-dimethoxy-3-nitro-10H-benzo[b]pyrido[2,3-e][1,4]oxazine (Compound TZ-2-69)

Compound TZ-2-69 was synthesized according to the reaction sequence shown in FIG. 3.

In the first step, 2-amino-3,5-dimethoxyphenol hydrochloride (300 mg, 1.5 mmol), 1-fluoro-2,4-dinitrobenzene (270 mg, 1.5 mmol), and sodium acetate (360 mg, 4.5 mmol) were dissolved in 5 mL of ethanol and 1 mL of water. The reaction mixture was stirred at room temperature for 4 hours. The precipitate was filtered and washed with ethanol and water, then purified by column chromatography on silica gel to give 2-((2,4-dinitrophenyl)amino)-3,5-dimethoxyphenol (Compound TZ-2-67) as a red solid (410 mg, 82%). ¹H NMR (DMSO-d₆): δ 3.72 (s, 3H), 3.76 (s, 3H), 6.22 (d, J=10.4 Hz, 1H), 6.60 (d, J=9.9 Hz, 1H), 8.19 (d, J=8.7 Hz, 1H), 8.89 (s, 1H), 9.42 (b, 1H), 9.87 (b, 1H).

In the second step, TZ-2-64 (300 mg, 0.90 mmol) and potassium carbonate (400 mg, 3 mmol) were mixed in 5 mL of dimethylformamide and heated at 120° C. for 5 hours. The reaction was quenched with cold water (10 mL) and extracted with ethyl acetate (3×10 mL), the combined organic layer was washed with water and concentrated under reduced pressure. After purification by column chromatography on silica gel 7,9-dimethoxy-3-nitro-10H-benzo[b]pyrido[2,3-e][1,4]oxazine (TZ-2-69) was obtained as a violet solid (110 mg, 42%). ¹H NMR (DMSO-d₆, 300 MHz): δ 3.68 (s, 3H), 3.80 (s, 3H), 6.02 (s, 1H), 6.24 (s, 1H), 6.72 (d, 1H, J=8.4 Hz), 7.28 (s, 1H), 7.67 (d, 1H, J=8.1 Hz), 8.78 (s, 1H).

### Example 24

### Synthesis of 3-methoxy-7-(trifluoromethyl)-10H-phenothiazine (Compound TZ5B-71)

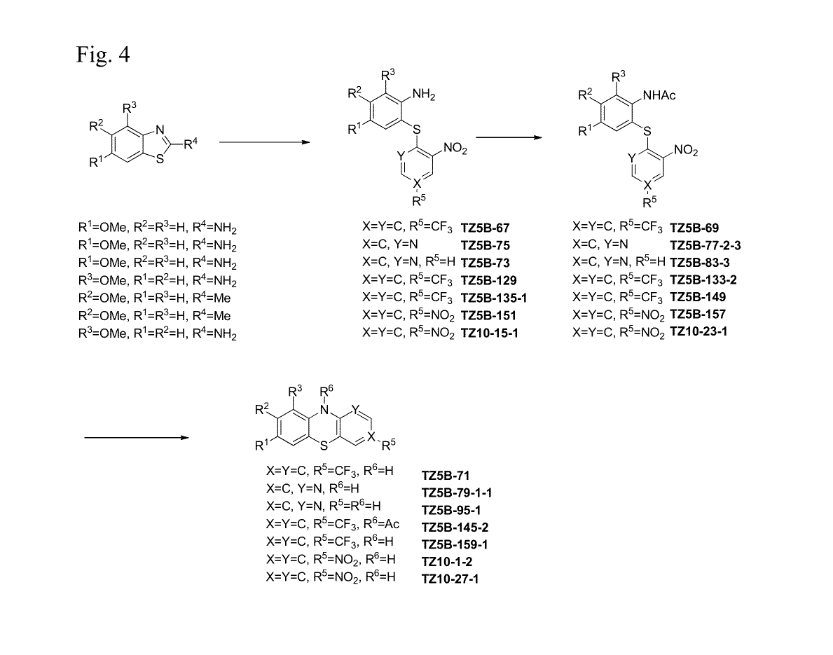

Compound TZ5B-71 was prepared according to the reaction scheme provided in FIG. 4.

Synthesis of TZ5B-67. Procedure A. To 10 mL of 50% aqueous sodium hydroxide was added 1.0 g (5.5 mmol) of 2-amino-6-methoxybenzothiazole followed by 3.0 mL of ethylene glycol. The resultant suspension was heated to reflux and refluxed overnight to give dark blue thick solution. This solution was added slowly (exothermic) to the solution of 1.25 g (5.5 mmol) 4-chloro-3-nitrobenzotrifluoride in approximately 30 mL of ethanol and acetic acid mixture (5:1). The pH of the reaction mixture was kept slightly acidic by adding acetic acid. Dirty yellow turbidity formation was observed. After stirring the resultant reaction mixture for 5 hrs, the solid formed was collected by filtration and the solid dissolved in dichloromethane and washed with water, brine and concentrated on rotovap to give 0.87 g (2.5 mmol, 46%) of brown sticky solid. ¹HNMR (CDCl₃): 8.54 (s, 1H), 7.59 (d, J=6.3 Hz, 1H), 7.01-6.98 (m, 3H), 6.85-6.82 (m, 1H), 3.99 (s, 2H), 3.75 (s, 3H).

Synthesis of TZ5B-69: In a 100 mL reaction flask was placed 835 mg (2.5 mmol) of TZ5B-67 and 26 mL of acetic anhydride was added followed by 5.3 mL of pyridine at room temperature. The resultant reaction mixture was stirred at room temperature until TLC (1:1 ethyl acetate in hexane) showed the disappearance of starting material and appearance of a new single spot. The reaction mixture was poured onto ice-water and stirred. Solid started to form in 5 min. The resultant mixture was stirred for additional 15-20 min and the solid was collected by filtration. The solid was washed with ethanol and water. Dissolved in dichloromethane and washed with water, brine and dried over anhydrous sodium sulfate, and concentrated on rotovap to give 855 mg (2.2 mmol, 88%) dirty yellowish green solid. ¹HNMR (CDCl₃): 8.56 (s, 1H), 8.37 (d, J=9.0 Hz, 1H), 7.72 (s br, 1H), 7.61 (d, J=8.7 Hz, 1H), 7.20-7.05 (m, 2H), 6.89 (d, J=9.0 Hz, 1H), 3.81 (s, 3H).

Synthesis of TZ5B-71. Procedure C. In a 250 mL round bottom flask was placed 855 mg (2.2 mmol) of TZ5B-69 and 75 mL of acetone was added and stirred. To the above solution, 2 equivalents of KOH in ethanol was added and the resultant reaction mixture was heated to reflux (4-24 hrs), cooled to room temperature and poured onto ice-water. Dark ash solid (400 mg, 1.3 mmol, 61%) formed was collected by filtration. ¹HNMR (CDCl₃): 7.22-7.19 (m, 2H), 6.85-6.60 (m, 4H), 5.84 (s br, 1H), 3.73 (s, 3H).

### Example 25

### Synthesis of 8-methoxy-5H-benzo[b]pyrido[4,3-e][1,4]thiazine (Compound TZ5B-79-1-1)

Compound TZ5B-79-1-1 was prepared according to the reaction scheme provided in FIG. 4.

Synthesis of TZ5B-75. Procedure A was followed starting with 1.0 g (5.5 mmol) of 2-amino-6-methoxybenzothiazole and 1.25 g (5.5 mmol) of 4-chloro-3-nitrobenzotrifluoride to give 0.99 g (3.6 mmol, 65%) of brown sticky solid. ¹HNMR spectra showed that the major compound was the desired product. ¹HNMR (CDCl₃): 9.37 (s, 1H), 8.41 (d, J=5.4 Hz, 1H), 7.01-6.94 (m, 2H), 6.85 (d, J=8.7 Hz, 1H), 6.77 (d, J=8.7 Hz, 1H), 3.75 (s, 3H).

Synthesis of TZ5B-77-2-3: Procedure B was followed starting with 920 mg (3.3 mmol) of TZ5B-75 to give brown oil which was purified on silica gel column chromatography (1:5 ethyl acetate:hexane to 1:4 ethyl acetate:hexane to 1:1 ethyl acetate:hexane to ethyl acetate) to give 534 mg (1.67 mmol, 51%) dark green sticky solid. ¹HNMR (CDCl₃): 9.40 (s, 1H), 8.43 (d, J=5.7 Hz, 1H), 8.35 (d, J=9.3 Hz, 1H), 7.64 (s br, 1H), 7.15 (dd, J=9.0, 3.0 Hz, 1H), 7.08 (d, J=3.3 Hz, 1H), 6.65 (d, J=6.0 Hz, 1H), 3.81 (s, 3H).

Synthesis of TZ5B-79-1-1. Procedure C was followed starting with 514 mg (1.6 mmol) of TZ5B-77-2-3 to give brown oil which was purified on silica gel column chromatography (1:1 ethyl acetate:hexane to ethyl acetate) to give 28 mg (0.12 mmol, 8%). ¹HNMR (CDCl₃): 8.14 (d, J=5.1 Hz, 1H), 7.98 (d, J=0.6 Hz, 1H), 7.00-6.85 (m, 1H), 6.60-6.55 (m, 2H), 6.48-6.44 (m, 1H), 6.36 (d, J=5.4 Hz, 1H), 5.90 (s br, 1H), 3.73 (s, 3H).

### Example 26

### Synthesis of 7-methoxy-10H-benzo[b]pyrido[2,3-e][1,4]thiazine (Compound TZ5B-95-1)

Compound TZ5B-95-1 was prepared according to the reaction scheme provided in FIG. 4.

Synthesis of TZ5B-73. Procedure A was followed starting with 1.0 g (5.5 mmol) of 2-amino-6-methoxybenzothiazole and 0.88 g (5.5 mmol) of 2-chloro-3-nitropyridine to give dark brown gel (1.12 g, 4.05 mmol, 73%). The crude material was used in the next step without further purification. ¹HNMR (CDCl₃) δ: 8.64 (dd, J=4.8, 1.8 Hz, 1H), 8.23 (dd, J=8.1, 1.8 Hz, 1H), 7.46 (dd, J=7.8, 4.8 Hz, 1H), 6.82-6.70 (m, 1H), 6.69-6.67 (m, 2H), 4.10 (br s, 2H), 3.60 (s, 3H).

Synthesis of TZ5B-83-3. Procedure B was followed starting with 1.12 g (4.05 mmol) of TZ5B-73 to give dark brown oil which was subjected to silica gel purification to give TZ5B-83-3 as solid (150 mg, 0.47 mmol, 12%). ¹HNMR (CDCl₃): δ 8.60-8.50 (m, 2H), 8.22-8.18 (m, 1H), 7.75 (br s, 1H), 7.08-7.05 (m 3H), 3.78 (s, 3H), 2.02 (s, 3H).

Synthesis of TZ5B-95-1. Procedure C was followed starting with 150 mg (0.47 mmol) of TZ5B-83-3 to give crude material which was purified on silica gel column to give brownish yellow solid (28 mg, 0.11 mmol, 23%). ¹HNMR(CDCl₃): δ 7.82 (dd, J=5.1, 1.3 Hz, 1H), 7.19-7.16 (m, 1H), 6.70-6.66 (m, 1H), 6.59-6.45 (m, 4H), 3.72 (s, 3H). MP: 179.5-180.7° C.

### Example 27

### Synthesis of 1-(1-methoxy-7-(trifluoromethyl)-10H-phenothiazin-10-yl)ethanone (Compound TZ5B-145-2)

Compound TZ5B-145-2 was prepared according to the reaction scheme provided in FIG. 4.

Synthesis of TZ5B-129. Procedure A was followed with 1.0 g (5.5 mmol) of 2-amino-4-methoxybenzothiazole and 1.25 g (0.83 mL, 5.55 mmol) of 4-chloro-3-nitrobenzotrifluoride to give TZ5B-129 as yellow solid (1.1 g, 3.21 mmol, 58%). The base used here was potassium hydroxide. ¹HNMR (CDCl₃): δ 8.54 (s, 1H), 7.56 (d, J=8.7 Hz, 1H), 7.10-6.90 (m, 3H), 6.85-6.75 (m, 1H), 4.46 (s, 2H), 3.92 (s, 3H).

Synthesis of TZ5B-133-2. Procedure B was followed starting with 0.9 g (2.61 mmol) of TZ5B-129 to give the solid which was subjected to silica gel purification to give TZ5B-133-2 as a yellow solid (431 mg, 1.11 mmol, 43%). ¹HNMR (CDCl₃): δ 8.47 (s, 1H), 7.60-7.50 (m, 1H), 7.40-7.25 (m, 1H), 7.19-7.12 (m, 3H), 6.85 (br s, 1H), 3.91 (s, 3H), 2.04 (s, 3H).

Synthesis of TZ5B-145-2. Procedure C was followed starting with 0.43 g (1.1 mmol) of TZ5B-133-2 to give yellow solid. The crude solid was purified on silica gel column to give TZ5B-145-2 as a yellow solid (114.5 mg, 0.34 mmol, 31%) ¹HNMR: (CDCl₃): δ 7.20-7.15 (m, 2H), 6.80-6.78 (m, 1H), 6.64-6.54 (m, 4H), 3.86 (s, 3H).

### Example 28

### Synthesis of 2-methoxy-7-(trifluoromethyl)-10H-phenothiazine (Compound TZ5B-159-1)

Compound TZ5B-159-1 was prepared according to the reaction scheme provided in FIG. 4.

Synthesis of TZ5B-135-1. Procedure A was followed with 1.0 g (5.5 mmol) of 5-methoxy-2-methylbenzothiazole and 0.83 mL (1.25 g, 5.5 mmol) 4-chloro-3-nitrobenzotrifluoride. The base used here was 10N sodium hydroxide solution. Crude material was purified on silica gel column (1:4 EtOAc/hexane) to give 0.71 g (2.06 mmol, 38%) of brown sticky solid. ¹HNMR (CDCl₃): δ 8.53 (s, 1H), 7.58 (d, J=8.7 Hz, 1H), 7.32 (d, J=7.5 Hz, 1H), 7.01 (d, J=8.7 Hz, 1H), 6.46-6.36 (m, 2H), 4.29 (s, 3H), 3.83 (s, 3H).

Synthesis of TZ5B-149. Procedure B was followed starting with 0.71 g (2.06 mmol) of TZ5B-135-1 to give TZ5B-149 as a yellow solid (0.61 g, 1.57 mmol, 76%). ¹HNMR (CDCl₃): δ 8.55 (s, 1H), 8.26 (s, 1H), 8.02 (s, 1H), 7.60 (d, J=8.4 Hz, 1H), 7.44 (d, J=8.4 Hz, 1H), 6.84 (d, J=8.1 Hz, 1H), 6.80-6.75 (m, 1H), 3.90 (s, 3H), 2.10 (s, 3H).

Synthesis of 20 (TZ5B-159-1). Procedure C was followed starting with 0.6 g (1.55 mmol) of TZ5B-149 to give 19 mg (0.06 mmol, 4%) of product after silica gel chromatographic purification (1:12 EtOAc/hexane to 1:10 EtOAc/hexane). ¹HNMR (CDCl₃): δ 9.83 (s, 1H), 8.43 (s, 1H), 7.52-7.40 (m, 2H), 6.79 (d, J=2.4 Hz, 1H), 6.59-6.55 (m, 1H), 3.76 (s, 3H).

### Example 29

### Synthesis of 2-methoxy-7-nitro-10H-phenothiazine (Compound TZ10-1-2)

Compound TZ 10-1-2 was prepared according to the reaction scheme provided in FIG. 4.

Synthesis of TZ5B-151. Procedure A was followed with 1.0 g (5.5 mmol) of 5-methoxy-2-methylbenzothiazole and 1.11 g (5.5 mmol) 1-chloro-2,4-dinitrobenzene. 10N sodium hydroxide solution was used as the base to give to give desired product in quantitative yield. ¹HNMR (CDCl₃): δ 9.11 (s, 1H), 8.17 (d, J=12.0 Hz, 1H), 7.31 (d, J=11.6 Hz, 1H), 7.05 (d, J=10.8 Hz, 1H), 6.45 (d, J=12.0 Hz, 1H), 6.38 (s, 1H), 4.26 (s, 2H), 3.84 (s, 3H).

Synthesis of TZ5B-157. Procedure B was followed starting with 5.5 mmol of crude TZ5B-151 to give yellow solid (0.6 g, 1.55 mmol, 30%). ¹HNMR (CDCl₃): δ 9.14 (s, 1H), 8.26 (s, 1H), 8.21-8.15 (m, 1H), 7.94 (s, 1H), 7.44 (d, J=11.6 Hz, 1H), 6.90 (d, J=12.0 Hz, 1H), 6.79 (d, J=7.6 Hz, 1H), 3.91 (s, 3H), 2.10 (s, 3H).

Synthesis of TZ10-1-2. Procedure C was followed starting with 0.6 g (1.65 mmol) of TZ5B-157 and 0.63 g (11.25 mmol) of potassium hydroxide in 150 mL ethanol. Solid formed was separated by filtration. The filtrate was concentrated on rotovap and purified on silica gel column to give the desired product with impurity. Therefore, this impure fraction from column was purified by crystallization from acetone and water to give TZ10-1-2 as dark purple solid (74 mg, 0.27 mmol, 17%). ¹HNMR (DMSO d₆): δ 9.52 (s, 1H), 7.85 (dd, J=9.0, 2.7 Hz, 1H), 7.74 (d, J=2.4 Hz, 1H), 6.86 (d, J=8.7 Hz, 1H), 6.69 (d, J=8.7 Hz, 1H), 6.47 (dd, J=8.7, 2.7 Hz, 1H), 6.32 (d, J=2.4 Hz, 1H), 3.68 (s, 3H).

### Example 30

### Synthesis of 1-methoxy-7-nitro-10H-phenothiazine (Compound TZ10-27-1)

Compound TZ 10-27-1 was prepared according to the reaction scheme provided in FIG. 4.

Synthesis of TZ10-15-1. Procedure A was followed with 1.0 g (5.5 mmol) of 2-amino-4-methoxybenzothiazole and 1.11 g (5.5 mmol) 1-chloro-2,4-dinitrobenzene. 50% aqueous potassium hydroxide was used as the base. The orange solid was collected by filtration, product extracted by washing the solid with dichloromethane and concentrating on the rotovap (1.56 g, 4.85 mmol, 88%). ¹HNMR (CDCl₃): δ 9.12 (d, J=3.6 Hz, 1H), 8.15 (dd, J=12.0, 3.6 Hz, 1H), 7.05-6.95 (m, 3H), 6.84-6.78 (m, 1H), 4.45 (s, 2H), 3.92 (s, 3H).

Synthesis of TZ10-23-1. Procedure B was followed starting with 1.5 g (4.8 mmol) of crude TZ10-15-1 to give yellow solid (0.86 g, 2.37 mmol, 55%). ¹HNMR (CDCl₃): δ 9.06 (s, 1H), 8.14 (d, J=12.0 Hz, 1H), 7.42-7.35 (m, 1H), 7.23-7.14 (m, 3H), 6.91 (s, 1H), 3.92 (s, 3H), 2.07 (s, 3H).

Synthesis of TZ10-27-1. Procedure C was followed starting with 0.46 g (1.26 mmol) of TZ5B-105 to give the desired product after chromatographic purification brown solid (90 mg, 0.33 mmol, 27%). ¹HNMR (CDCl₃): δ 7.80-7.60 (m, 1H), 7.78 (s, 1H), 6.85-6.78 (m, 1H), 6.74 (s, 1H), 6.63 (d, J=8.4 Hz, 1H), 6.55-6.48 (m, 2H), 3.87 (s, 3H).

### Example 31

### Synthesis of 1-(phenazin-5(10H)-yl)ethanone (Compound TZ16-147-2) and 1,1′-(phenazine-5,10-diyl)diethanone (Compound TZ16-147-3)

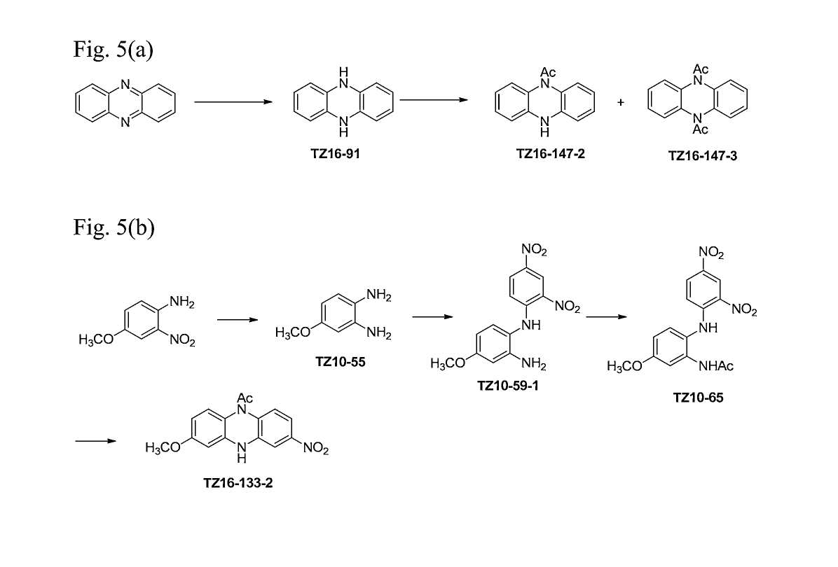

Compounds TZ16-147-2 and TZ16-147-3 were prepared according to the reaction scheme provided in FIG. 5(a).

Synthesis of TZ16-91. Approximately 180 mg (1.0 mmol) of phenazine was placed in a 100 mL round bottom flask and 5.0 mL of ethanol was added and the solution was heated to boiling. Sodium hydrosulfate (1.7 g, 10.0 mmol) in 20 mL water was added to the above boiling solution. The solid formation was observed immediately. The solid formed was collected by filtration and dried under vacuum to give 151 mg (0.82 mmol, 82%) of TZ16-91 as white solid. ¹HNMR (CDCl₃): δ 8.30-8.24 (m, 4H), 7.90-7.80 (m, 4H).

Synthesis of TZ16-147-2 and TZ16-147-3. To approximately 0.55 g (3.0 mmol) of TZ16-141 in a 50 mL reaction flask was added 6.0 mL of acetic anhydride and the resulting suspension was heated to 135° C. at which point the reaction mixture turned clear. The reaction was heated at this temperature for 10 min and cooled to room temperature, treated with water and extracted with dichloromethane, concentrated on rotovap, and purified on silica gel column to give TZ16-147-2 and TZ-147-3 (600 mg, 2.2 mmol, 75%) as white solids. ¹HNMR of TZ16-147-2 (DMSO d₆): δ 8.93 (s, 1H), 7.34 (d, J=8.0 Hz, 2H), 7.10 (t, J=8.0 Hz, 2H), 6.92-6.87 (m, 4H), 2.10 (s, 3H). ¹HNMR of TZ16-147-3 (CDCl₃): δ 7.53 (s br, 4H), 7.28-7.23 (m, 4H), 2.35 (s, 6H).

### Example 32

### Synthesis of 2-methoxy-8-nitro-5,10-dihydrophenazine (Compound TZ16-133-2)

Compound TZ16-133-2 was prepared according to the reaction scheme provided in FIG. 5(b).

Synthesis of TZ10-55. Approximately 360 mg (2.14 mmol) of 4-methoxy-2-nitroanniline was placed in a hydrogenator and 65 mL ethanol was added. A pinch of 10% palladium on activated carbon was added and the reaction mixture hydrogenated at room temperature under 50 Psi pressure for 24 hrs. The reaction mixture was filtered and concentrated on rotovap to give TZ10-55 as dark purple oil (294 mg, 2.12 mmol, 99%). ¹HNMR (CDCl₃): δ 6.64 (d, J=8.7 Hz, 1H), 6.35-6.30 (m, 1H), 6.26 (d, J=8.7 Hz, 1H), 3.52 (s br, 2H), 3.07 (s br, 2H).

Synthesis of TZ10-59-1. To approximately 286 mg (2.0 mmol) of TZ10-55 in 2.0 mL DMF was added 404 mg (2.0 mmol) followed by 828 mg of potassium carbonate. The resultant reaction mixture was heated to 100° C. and stirred overnight. The reaction mixture was cooled to room temperature and diluted with ethyl acetate. Ethyl acetate layer was washed with saturated aqueous solution of sodium bicarbonate, water, and brine, and dried over anhydrous sodium sulfate. Chromatographic purification give 211 mg (0.69 mmol, 35%) of TZ10-59-1 as rust solid. ¹HNMR (CDCl₃): δ 9.43 (s br, 1H), 9.18 (s, 1H), 8.16 (d, J=8.7 Hz, 1H), 7.01 (d, J=9.6 Hz, 1H), 6.80 (d, J=9.3 Hz, 1H), 6.50-6.30 (m, 2H), 3.81 (s, 5H).

Synthesis of TZ10-65. To 175 mg (0.58 mmol) of TZ10-59-1 was added 12 mL of acetic anhydride followed by 2.5 mL of pyridine. The resultant reaction mixture was stirred at room temperature overnight. Reaction mixture was poured onto crushed-ice. Yellow solid formed was collected by filtration and dissolved in dichloromethane. Dichloromethane layer was washed with water, brine, dried over anhydrous sodium sulfate and concentrated on rotovap. The crude material was purified on silica gel column to give 99 mg (0.28, 48%) of TZ10-65 as a yellow solid. ¹HNMR (CDCl₃): δ 9.52 (s, 1H), 9.19 (s, 1H), 8.17 (d, J=9.3 Hz, 1H), 7.75 (s, 1H), 7.20-7.10 (m, 2H), 6.79 (d, J=10.2 Hz, 2H), 3.87 (s, 3H), 2.13 (s, 3H).

Synthesis of TZ16-133-2. To 369 mg (1.1 mmol) of TZ16-85 and T6-119 in DMF was added >4 equivalents of potassium carbonate (4.4 mmol) and the reaction mixture was heated to 125° C. for 4 d. The reaction was not complete based on TLC, however, reaction mixture was cooled to room temperature and diluted with ethyl acetate and treated with water. Ethyl acetate layer separated and aqueous layer extracted thrice with ethyl acetate. Combined organic extracts were concentrated on rotovap and purified on silica gel column to give 36 mg (0.12 mmol, 11%) of yellow solid. ¹HNMR (CDCl₃): δ 9.80 (s, 1H), 9.17 (s, 1H), 8.17 (d, J=9.2 Hz, 1H), 7.59 (d, J=9.2 Hz, 1H), 7.10-7.01 (m, 2H), 6.93 (d, J=9.6 Hz, 1H), 6.87 (s, 1H), 3.81 (s, 3H), 2.13 (s, 3H).

### Example 33

### Thioflavin T fluorescence assay for α-synuclein fibrils binding

α-Synuclein recombinant protein was produced in E. coli. BL21(DE3)RIL E. coli were transformed with a pRK172 bacterial expression plasmid containing the human α-synuclein coding sequence. Freshly transformed BL21 colonies were inoculated into 2 L baffled flasks containing 250 mL sterilized TB (1.2% bactotryptone, 2.4% yeast extract, 0.4% glycerol, 0.17 M KH₂PO₄, 0.72 M K₂HPO₄) with 50 μg/ml ampicillin, and incubated overnight at 37° C. with shaking Overnight cultures were pelleted by centrifugation at 3,900×g for 10 min at 25° C. Bacterial pellets were resuspended in 20 mL osmotic shock buffer (30 mM Tris-HCl, 2 mM EDTA, 40% Sucrose, pH 7.2) by gentle vortexing and incubated at room temperature for 10 minutes. The cell suspension was then centrifuged at 8,000×g for 10 min at 25° C. and the pellet was resuspended in 22.5 mL cold H₂O before adding 9.4 μL 2 M MgCl₂ to each tube. The suspension was incubated on ice for 3 min prior to centrifugation at 20,000×g for 15 min at 4° C. The supernatant was transferred to a fresh tube, streptomyocin was added to a final concentration of 10 mg/mL, and then centrifuged at 20,000×g for 15 min at 4° C. The supernatant from this step was collected and dithiothreitol (DTT) and Tris-HCl were added to final concentrations of 1 mM and 20 mM respectively, before boiling for 10 min to precipitate heat-sensitive proteins, which were pelleted at 20,000×g for 15 minutes at 4° C. The supernatant was collected and filtered through a 0.45 μm surfactant free cellulose acetate filter (Corning) before loading onto a 1 mL DEAE Sepharose column equilibrated in 20 mM Tris-HCl pH 8, 1 mM EDTA, and 1 mM DTT. The DEAE column was washed with 20 mM Tris-HCl pH 8, 1 mM EDTA, 1 mM DTT before eluting α-synuclein protein in 20 mM Tris-HCl, pH 8, buffer with 1 mM EDTA, 1 mM DTT and 0.3 M NaCl. The purified α-synuclein protein was dialyzed overnight in 10 mM Tris-HCl, pH 7.6, 50 mM NaCl, 1 mM DTT. Preparations contained greater than 95% α-synuclein protein as determined by SDS-PAGE and BCA assay with a typical yield of 30 mg protein per 250 ml culture.

The purified, recombinant α-synuclein monomer (2 mg/mL) was incubated in Tris-HCl (20 mM) and NaCl (100 mM) while shaking at 1000 rpm in an Eppendorf Thermomixer in a 37° C. temperature-controlled room for 72 hours. To determine the concentration of fibrils, the reaction mixture (100 μL) was centrifuged at 18,000×g for 10 minutes to separate fibrils from monomer. The α-synuclein monomer and other soluble proteins in the supernatant were removed, and the fibril pellet was resuspended in 100 μL solution of Tris-HCl (20 mM) and NaCl (100 mM). This fibril suspension was used in a bicinchoninic acid (BCA) protein assay along with a bovine serum albumin (BSA) standard curve to determine the concentration of fibrils in the 72 hour fibril reaction mixture.

To prepare the fibrils for performing binding assays, the fibril reaction mixture prepared above was centrifuged at 18,000×g for 10 minutes. The supernatant was discarded and the fibril pellet was resuspended in Tris-HCl buffer (30 nM, pH=7.4) to achieve the desired concentration (3 or 6 μM) of fibrils for use in the assay.

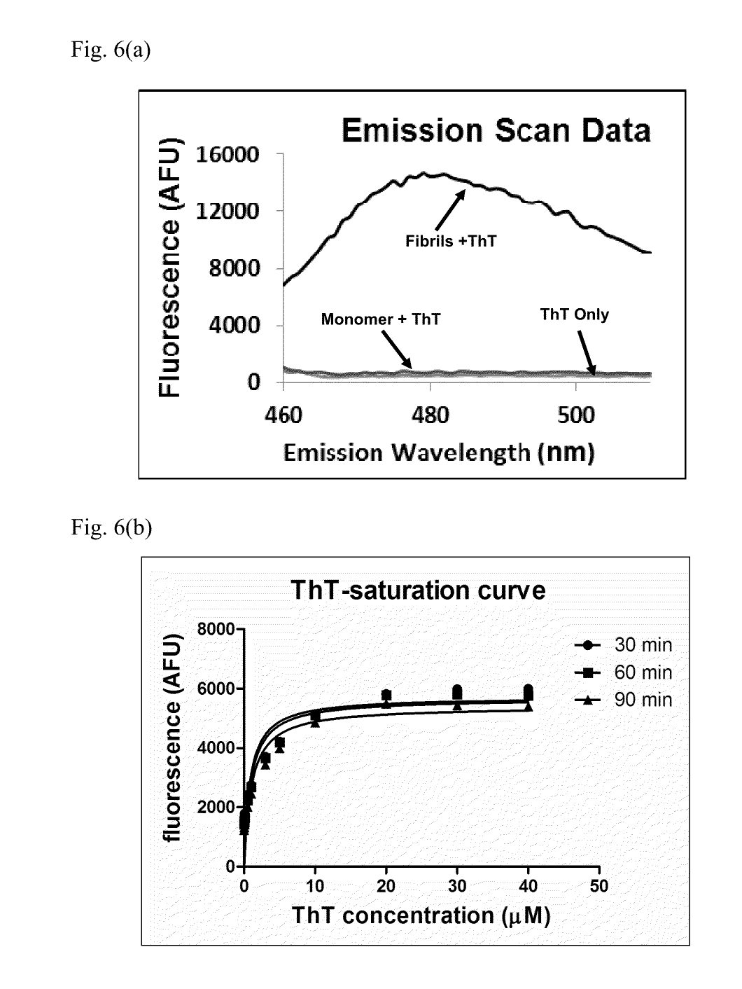

The ThT solution (6 μM) in Tris-HCl buffer (30 nM, pH=7.4, 40 μL) was added to each of three cells in a 96 cell plate for fluorescence detection containing α-synuclein fibrils suspension (3.0 μM) in the Tris-HCl buffer (30 nM, pH=7.4, 40 μL). The mixture was incubated at room temperature for 1 hour on the shaking plate. The reaction plate was scanned by the excitation wavelength range from 430 to 465 nm. The maximum excitation wavelength (λₑₓ) was determined according to the fluorescent intensity-excitation wavelength curve. At (λₑₓ), the emission wavelength was scanned to get maximum emission wavelength (λₑₘ). Then λₑₓ and kₑₘ for the free ThT and ThT-monomeric α-synuclein was determined by the procedure described above. See FIG. 6(a) for fluorescence emission spectra scan data at λₑₓ=440 nm and the ThT saturation curve, respectively. See FIG. 6(b) for the saturation curve of ThT (3 μM) for α-synuclein fibrils (1.5 μM) in Tris buffer (30 mM, pH=7.4) at different incubation times: 30 min (circle), 60 min (square), 90 min (triangle) at room temperature. The K_{d} for ThioT binding to fibrils was 948 nM and the Bₘₐₓ was 5672 afu.

ThT solutions of various concentration from 10 nM to 40 μM in Tris-HCl buffer (30 nM, pH=7.4, 40 μL) were added to a 96 cell plate containing α-synuclein fibrils (3.0 μM) in the Tris-HCl buffer (30 nM, pH=7.4, 40 μL). The mixture was incubated at room temperature for 1 hour on the shaking plate. The fluorescent intensity for each cell was measured by the fluorescence reader at λₑₓ and kₑₘ. The ThT-α-synuclein fibrils saturation curve and K_{d} value were produced by the software Prism 5. The K_{d} value for ThT binding to α-synuclein fibrils has been determined to be 948±271 nM.

Once the ThT-α-synuclein saturation binding curve and dissociation constant (K_{d}) were determined the competitive assay for determining the binding affinity of various test compounds was conducted. ThT solution (12 μM) in Tris-HCl buffer (30 nM, pH=7.4, 20 μL) was added to a 96 cell plate containing α-synuclein fibrils (6.0 μM) in the Tris-HCl buffer (30 nM, pH=7.4, 20 μL) and test compounds listed in Table 1 at various concentrations (from 1 nM to 10 μM) in Tris-HCl buffer (30 nM, pH=7.4, 40 μL) with 10% dimethyl sulfoxide. The mixture was incubated at room temperature for 60 minutes on the shaking plate. The fluorescent intensity for each cell was measured by the fluorescence reader at λₑₓ and kₑₘ. The Kᵢ value for each compound was calculated by the corresponding inhibition curve.

Table 1 shows the α-synuclein binding affinity data for a series of phenothiazines and phenoxazines, as determined by the ThT competition assay. The inhibitor that binds with highest affinity to α-synuclein fibrils has the lowest dissociation constant (Kᵢ). As can be seen from Table 1, the 3-nitro-7-methoxyphenothiazine (SIL5) had a Kᵢ of 32.1±1.3 nM, and was one of the most potent ligand in the series. Compound TZ-2-33 was found to be a potent ligand with a Kᵢ value of 25.7. The 3-nitro-8-methoxy phenoxazine TZ-2-39 was also a very potent ligand, with a Kᵢ value of 9.5. Of note is that substitution of the C-1 carbon in the aromatic ring for nitrogen yielded potent ligands TZ-2-45 and TZ-2-48 which had Log P values in the desired 1-3 range.

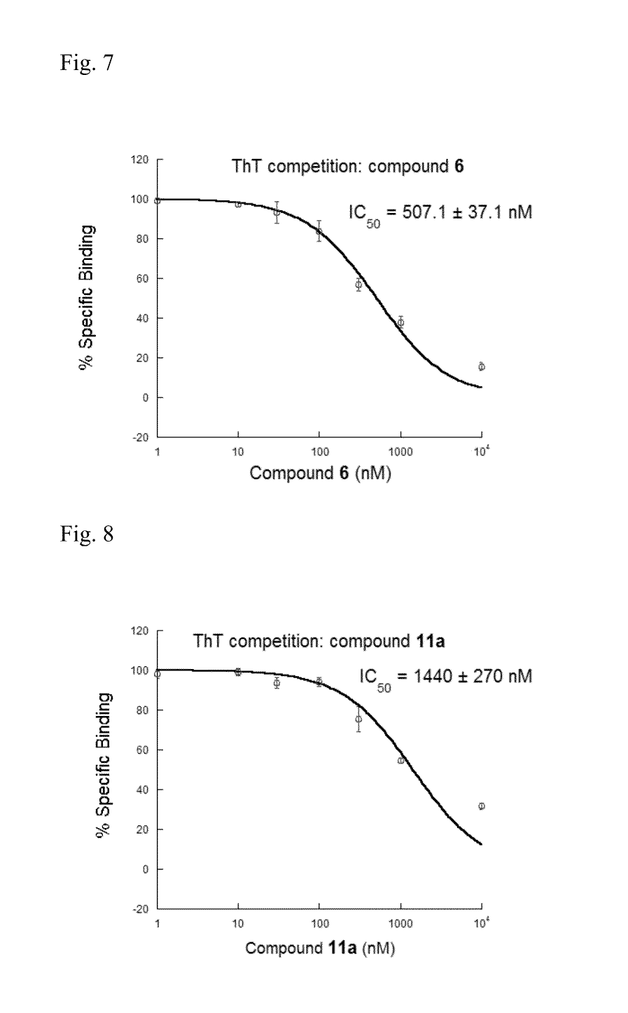

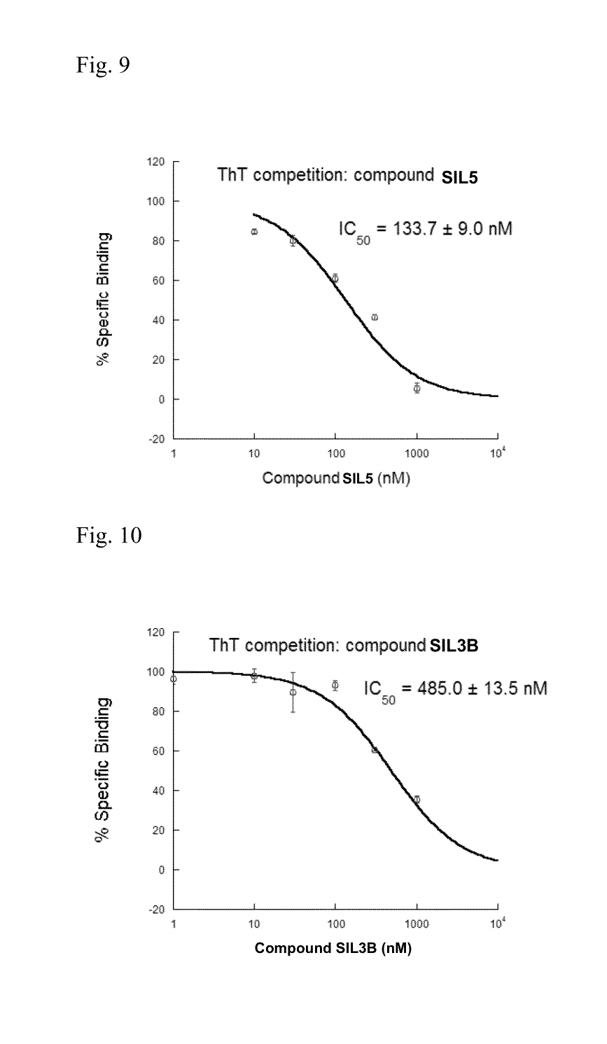

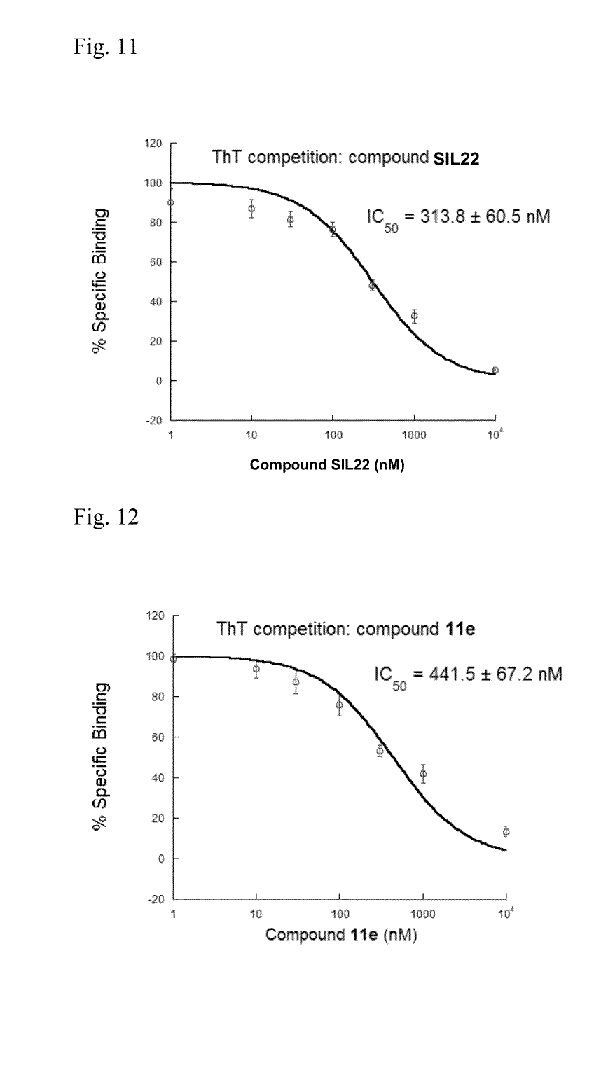

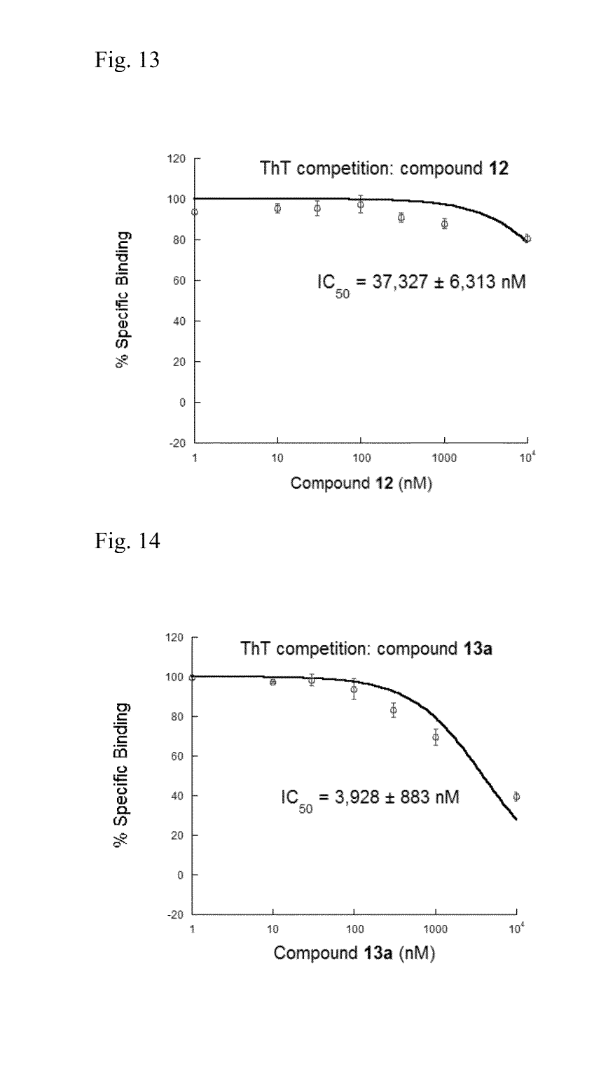

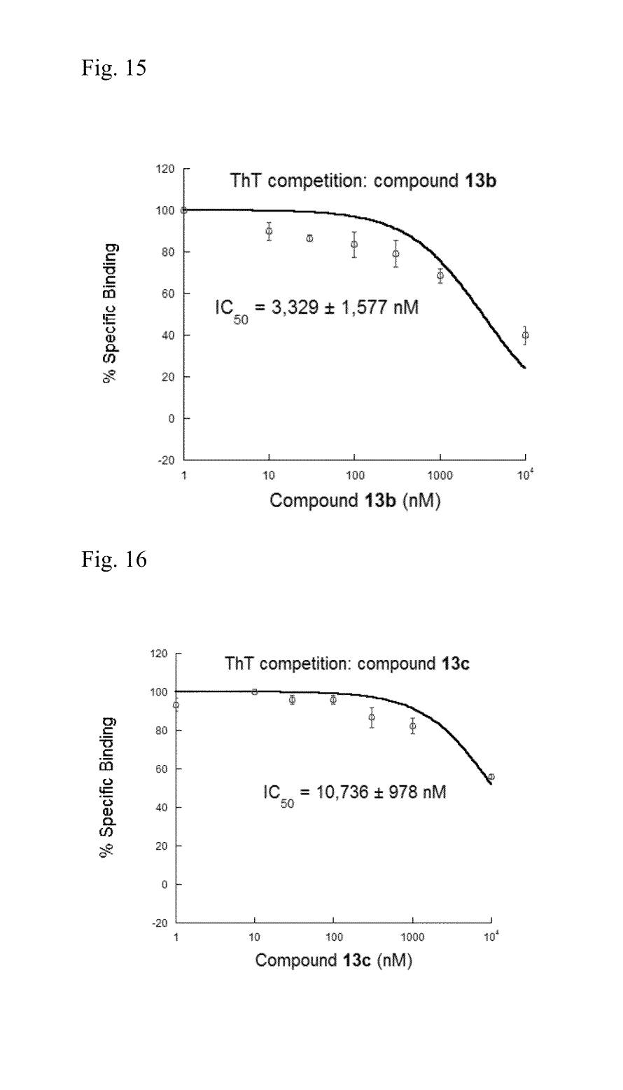

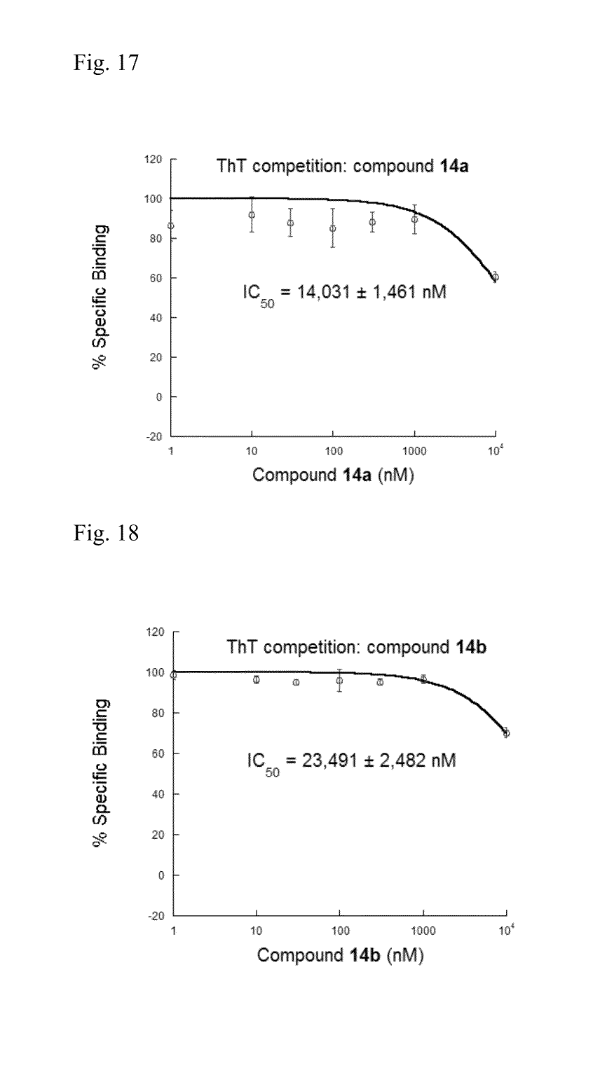

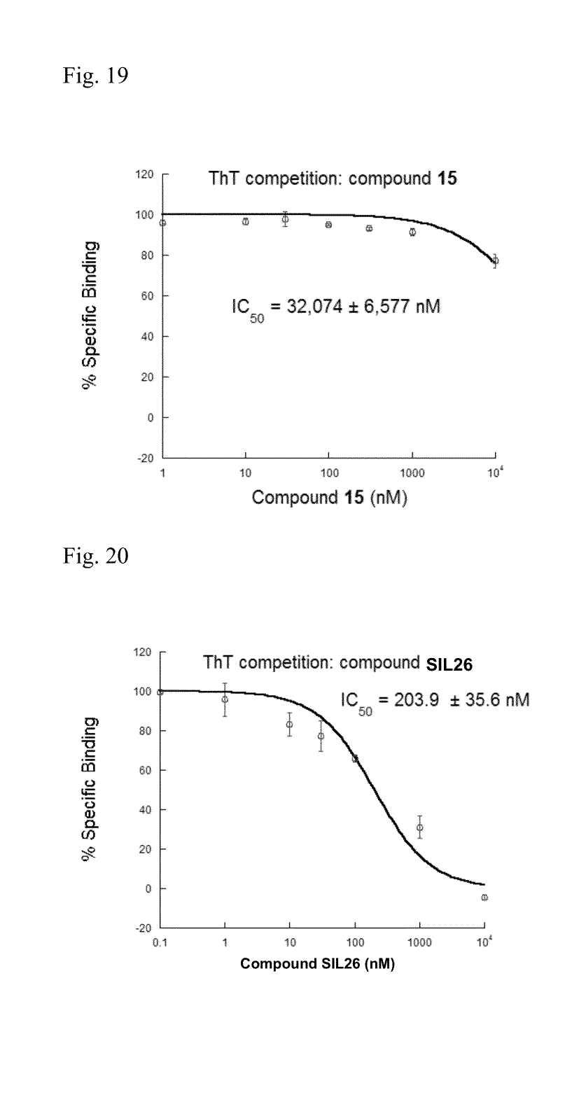

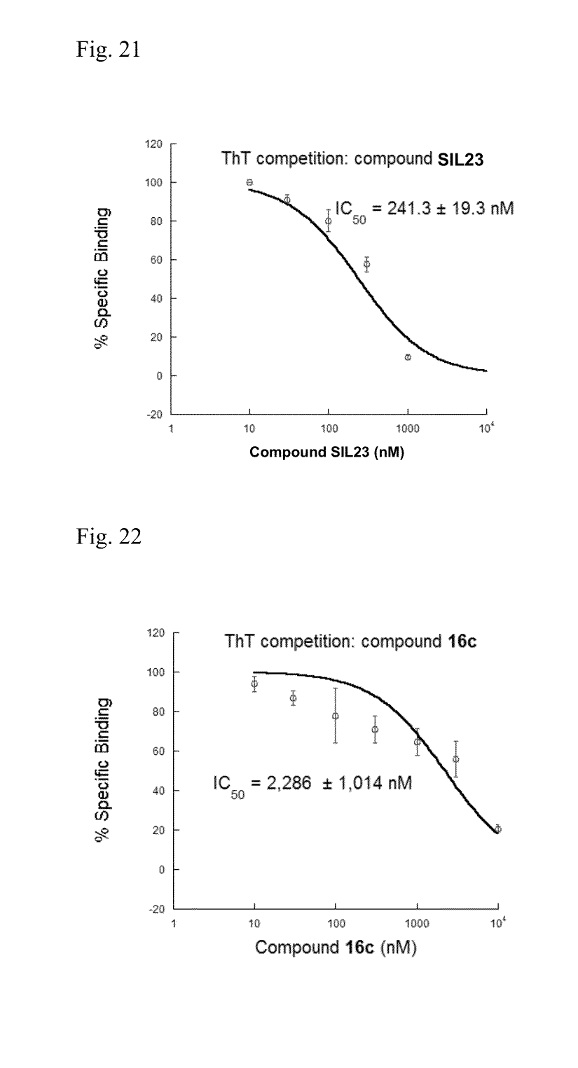

IC₅₀ values for each compound were determined by fitting the data to the equation Y=Bottom+(Top-Bottom)/(1+10^{(X-LogIC50)}) using nonlinear regression by Kaleidagraph software, where Top and Bottom are the Y values for the top and bottom plateaus of the binding curve. The Kᵢ values were derived from the IC₅₀ values using the Cheng-Prusoff equation: Kᵢ=IC₅₀/(1+[ThT]/K_{d}). See FIGS. 7-22 for the inhibition curves for each compound in the ThioT competitive binding assay.

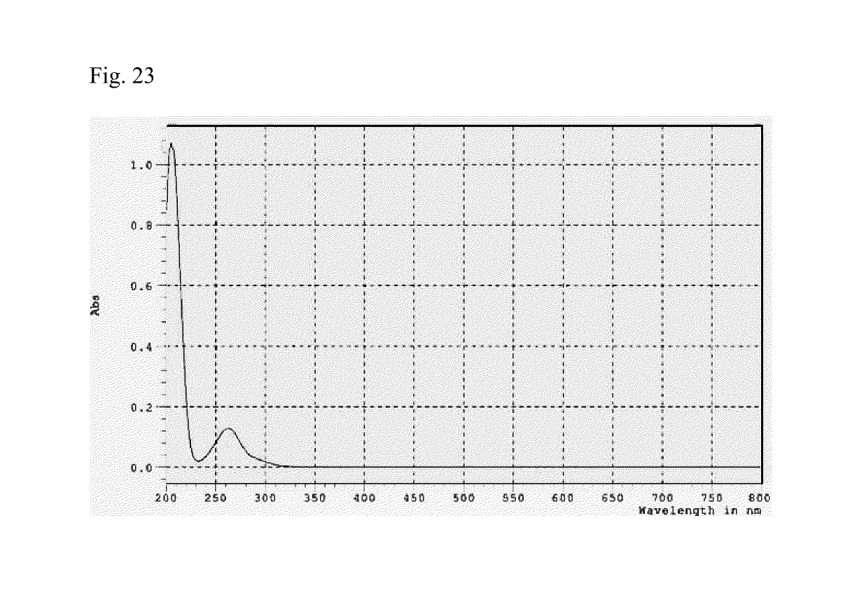

Although fluorescence quenching can potentially interfere with measurement of competitive binding, the data for the individual compounds closely fit a competitive binding model. Absorbance spectra were measured at the IC₅₀ concentration for each compound. Absorbance was less than 0.001 in the range of 400-500 nM for all of the compounds, indicating that absorbance at the excitation or emission wavelengths did not interfere with the fluorescence assay. For example, see FIG. 23, which is the uv/vis absorbance spectrum for Compound SIL5 at the IC₅₀ concentration of 133 nM.

TABLE 1
Binding affinity (Kᵢ)^{a} of certain phenothiazine, phenoxazine, and phenazine derivatives
determined using ThT competitive assay

| Compound | | | | |
|---|---|---|---|---|
| No. | Compound | (Kᵢ)^{a} (nM) | IC₅₀ (nM) | LOG P^{b} |
| 6 | 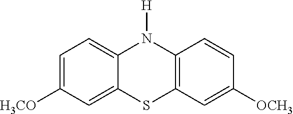 [SMILES: COc1ccc2c(c1)Sc1cc(OC)ccc1N2] | 121.8 ± 5.1 | 507.1 ± 37.1 | 3.98 |
| 11a | 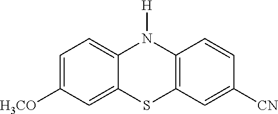 [SMILES: COc1ccc2c(c1)Sc1cc(C#N)ccc1N2] | 346 ± 37 | 1440 ± 270 | 3.50 |
| SIL5 | 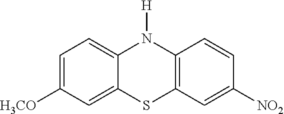 [SMILES: COc1ccc2c(c1)Sc1cc([N+](=O)[O-])ccc1N2] | 32.1 ± 1.3 | 133.7 ± 9.0 | 3.79 |
| SIL3B | 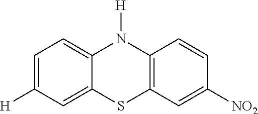 [SMILES: O=[N+]([O-])c1ccc2c(c1)Sc1ccccc1N2] | 116.5 ± 1.9 | 485.0 ± 13.5 | 3.88 |
| SIL22 | 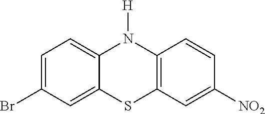 [SMILES: O=[N+]([O-])c1ccc2c(c1)Sc1cc(Br)ccc1N2] | 75.3 ± 8.4 | 313.8 ± 60.5 | 4.65 |
| 11e | 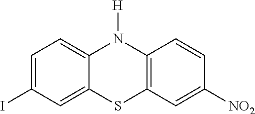 [SMILES: O=[N+]([O-])c1ccc2c(c1)Sc1cc(I)ccc1N2] | 106.0 ± 9.3 | 441.5 ± 67.2 | 4.91 |
| 12 | 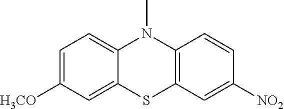 [SMILES: COc1ccc2c(c1)Sc1cc([N+](=O)[O-])ccc1N2C] | >500 | 37327 ± 6313 | 4.14 |
| 13a | 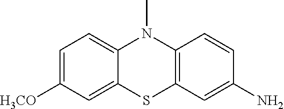 [SMILES: COc1ccc2c(c1)Sc1cc(N)ccc1N2C] | >500 | 3928 ± 883 | 3.12 |
| 13b | 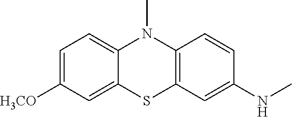 [SMILES: CNc1ccc2c(c1)Sc1cc(OC)ccc1N2C] | >500 | 3329 ± 1577 | 3.78 |
| 13c | 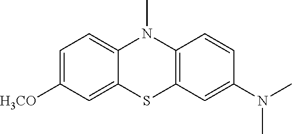 [SMILES: COc1ccc2c(c1)Sc1cc(N(C)C)ccc1N2C] | >500 | 10736 ± 978 | 4.52 |
| 14a | 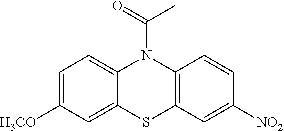 [SMILES: COc1ccc2c(c1)Sc1cc([N+](=O)[O-])ccc1N2C(C)=O] | >500 | 14031 ± 1461 | 1.86 |
| 14b | 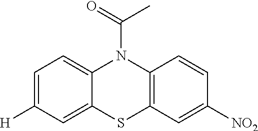 [SMILES: CC(=O)N1c2ccccc2Sc2cc([N+](=O)[O-])ccc21] | >500 | 23491 ± 2482 | 2.76 |
| 15 | 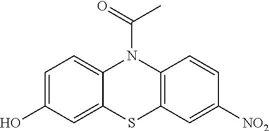 [SMILES: CC(=O)N1c2ccc(O)cc2Sc2cc([N+](=O)[O-])ccc21] | >500 | 32074 ± 6577 | 1.93 |
| SIL26 | 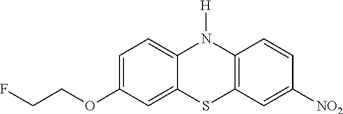 [SMILES: O=[N+]([O-])c1ccc2c(c1)Sc1cc(OCCF)ccc1N2] | 49.0 ± 4.9 | 203.9 ± 35.6 | 4.02 |
| SIL23 | 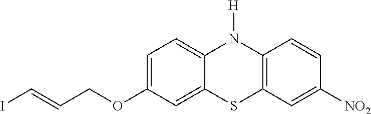 [SMILES: O=[N+]([O-])c1ccc2c(c1)Sc1cc(OC/C=C/I)ccc1N2] | 57.9 ± 2.7 | 241.3 ± 19.3 | 5.72 |
| 16c | 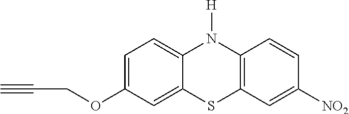 [SMILES: C#CCOc1ccc2c(c1)Sc1cc([N+](=O)[O-])ccc1N2] | >500 | 2286 ± 1014 | 3.83 |
| TZ5B-71 | 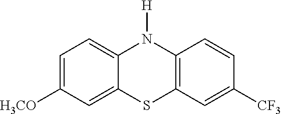 [SMILES: COc1ccc2c(c1)Sc1cc(C(F)(F)F)ccc1N2] | 178.34 | 742.7 ± 1.1 | 4.64 |
| TZ5B-79-1-1 | 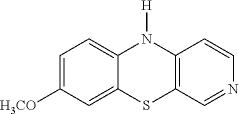 [SMILES: COc1ccc2c(c1)Sc1cnccc1N2] | 535.71 | 2231 ± 1.2 | 2.55 |
| TZ5B-95-1 | 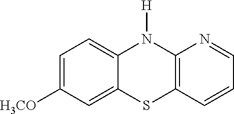 [SMILES: COc1ccc2c(c1)Sc1cccnc1N2] | 1590.6 | 6624 ± 1.4 | 2.71 |
| TZ5B-145-2 | 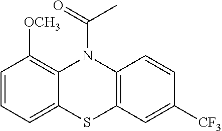 [SMILES: COc1cccc2c1N(C(C)=O)c1ccc(C(F)(F)F)cc1S2] | 251.7 | 1048 ± 1.9 | 2.76 |
| TZ5B-159-1 | 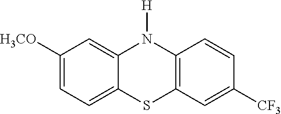 [SMILES: COc1ccc2c(c1)Nc1ccc(C(F)(F)F)cc1S2] | 259.8 | 1082 ± 2.4 | 4.98 |
| TZ10-1-2-1 | 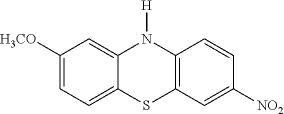 [SMILES: COc1ccc2c(c1)Nc1ccc([N+](=O)[O-])cc1S2] | 36.50 | 152 ± 1.1 | 4.14 |
| TZ10-27-1 | 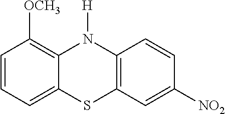 [SMILES: COc1cccc2c1Nc1ccc([N+](=O)[O-])cc1S2] | 342.41 | 1426 ± 1.2 | 3.78 |
| TZ-2-33 | 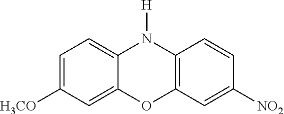 [SMILES: COc1ccc2c(c1)Oc1cc([N+](=O)[O-])ccc1N2] | 25.7 | 106.9 ± 1.0 | 3.9 |
| TZ-2-39 | 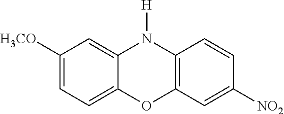 [SMILES: COc1ccc2c(c1)Nc1ccc([N+](=O)[O-])cc1O2] | 9.5 | 39.6 ± 1.2 | 4.24 |
| TZ-2-45 | 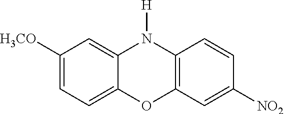 [SMILES: COc1ccc2c(c1)Nc1ccc([N+](=O)[O-])cc1O2] | 17.5 | 72.9 ± 1.1 | 3 |
| TZ-2-48 | 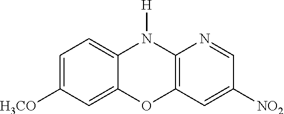 [SMILES: COc1ccc2c(c1)Oc1cc([N+](=O)[O-])cnc1N2] | 26.8 | 111.8 ± 1.1 | 2.66 |
| TZ-2-52 | 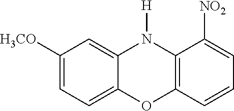 [SMILES: COc1ccc2c(c1)Nc1c(cccc1[N+](=O)[O-])O2] | 120.3 | 500.9 ± 1.1 | 4.35 |
| TZ-2-54 | 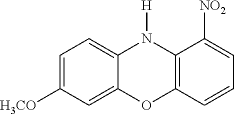 [SMILES: COc1ccc2c(c1)Oc1cccc([N+](=O)[O-])c1N2] | 164.9 | 686.9 ± 1.2 | 4.01 |
| TZ-2-65 | 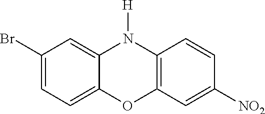 [SMILES: O=[N+]([O-])c1ccc2c(c1)Oc1ccc(Br)cc1N2] | 57.1 | NA | 5.19 |
| TZ-2-69 | 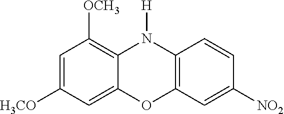 [SMILES: COc1cc(OC)c2c(c1)Oc1cc([N+](=O)[O-])ccc1N2] | NA | NA | 3.67 |
| TZ16-149-1 | 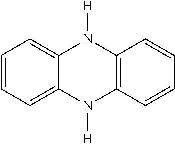 [SMILES: c1ccc2c(c1)Nc1ccccc1N2] | NA | NA | NA |
| TZ16-147-2 | 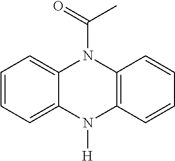 [SMILES: CC(=O)N1c2ccccc2Nc2ccccc21] | NA | NA | NA |
| TZ16-147-3 | 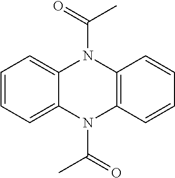 [SMILES: CC(=O)N1c2ccccc2N(C(C)=O)c2ccccc21] | >1000 | NA | NA |
| TZ16-133-2 | 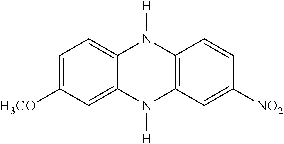 [SMILES: COc1ccc2c(c1)Nc1cc([N+](=O)[O-])ccc1N2] | >1000 | NA | NA |

^{a}Kᵢ values (mean ± SEM) were determined in at least three experiments.
^{b}Calculated value at pH 7.4 with ACD/Lab, version 7.0, (Advanced Chemistry Development, Inc., Canada).

### Example 34

### Preparation of radioligand [¹¹C]TZ-2-39

1.2 mg of 2-hydroxy-7-nitro-10H-phenoxazine was freshly dissolved in 0.2 mL of DMF followed by addition of 50.0 μL of NaH solution (10 mg/mL DMF). [¹¹C]CH₃I was bubbled into this reaction mixture for 4-7 minutes. After the complete transfer of radioactivity, the sealed reaction vial was heated at 90° C. for 5 min. The reaction mixture was removed from the oil bath and then quenched with 1.5 mL of HPLC mobile phase via the addition loop. The radioactive reaction mixture was then injected onto a reverse-phase Phenomenex prodigy C18 semi-preparative HPLC column (250×10 mm, 10 g) for purification. The HPLC mobile phase solution was 60% acetonitrile, 40% 0.1 M ammonium formate buffer solution, (pH 4.5), with UV wavelength at 276 nm, and a flow rate of 3.5 mL/min. Under these conditions, [¹¹C]TZ-2-39 was collected between 15.0-16.5 min in a sterile vial with water (50 mL). The radioactive solution was then passed through a C18 SepPak cartridge (Waters, WAT020515) which traps the [¹¹C]TZ-2-39 compound. The radioactive product was eluted with absolute ethanol (0.3 mL) and formulated with saline (10% ethanol solution in saline). The final dose was filtered using a sterile 0.22 μm pyrogen-free Millipore filter (Millipore Corp., Billerica, Mass.) into the dose vial for animal studies and quality control analysis.

### Example 35

### Radiosynthesis of [¹²⁵I]SIL23

In order to obtain ¹²⁵I radiolabeled compound SIL23, its trans-vinyl bromide precursor (SIL28)was needed. In this synthetic route, a mixture of (E) and (Z) 1,3-dibromoprop-1-ene was used to couple with phenothiazine precursor to obtain isomers of TZ17-16 as shown in the reaction below and described below.

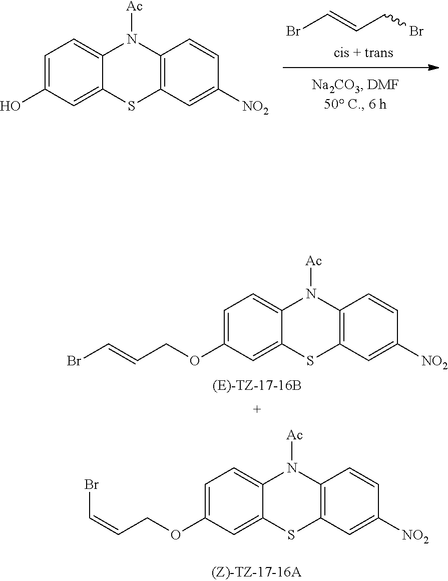 [SMILES: CC(=O)N1c2ccc(O)cc2Sc2cc([N+](=O)[O-])ccc21 ; Br/C=C/CBr ; CC(=O)N1c2ccc(OC/C=C/Br)cc2Sc2cc([N+](=O)[O-])ccc21 ; CC(=O)N1c2ccc(OC/C=C\Br)cc2Sc2cc([N+](=O)[O-])ccc21]

To a reaction vial containing 1-(3-hydroxy-7-nitro-10H-phenothiazin-10-yl)ethanone (40.0 mg, 0.132 mmol, 1.0 eq) and Na₂CO₃ (28.0 mg, 0.264 mmol) in 2 mL DMF was added 1,3-dibromoprop-1-ene (32.7 mg, 0.165 mmol). The reaction mixture was stirred at 50° C. for 6 hours. After aqueous work-up, the residue was purified by flash column chromatography (hexane/ethyl acetate 4:1 to 1:1). 26 mg TZ17-16A (72% yield) and 12 mg of TZ17-16B (33% yield) was purified. For TZ17-16A: ¹H NMR (400 MHz, CDCl₃): δ 8.21 (s, 1H), 8.09 (d, J=8.8 Hz, 1H), 7.68 (d, J=8.4 Hz, 1H), 7.31 (d, J=8.4 Hz, 1H), 6.99 (s, 1H), 6.84 (d, J=7.6 Hz, 1H), 6.42-6.37 (m, 2H), 4.71 (d, J=5.2 Hz, 2H); For TZ17-16B: ¹H NMR (400 MHz, CDCl₃): δ 8.22 (s, 1H), 8.16 (d, J=8.8 Hz, 1H), 7.68 (d, J=8.4 Hz, 1H), 7.31 (d, J=8.4 Hz, 1H), 6.97 (s, 1H), 6.85 (d, J=7.6 Hz, 1H), 6.22-6.43 (d, J=14.0 Hz, 1H), 6.41-6.37 (m, 1H), 4.42 (d, J=5.2 Hz, 2H).

The (E) and (Z) isomers were carefully isolated by flash column chromatography as further described and identified by ¹H NMR. TZ17-16B, the (E)-isomer was then subjected to hydrolysis under DBU, and SIL28 was obtained with good chemical yield. 12.0 mg of (E)-1-(3-((3-bromoallyl)oxy)-7-nitro-10H-phenothiazin-10-yl)ethanone (TZ17-16B) was stirred in CH₂Cl₂ (1.5 mL) and DBU (0.1 mL) under 90° C. for 1 hour. Solvents were removed under reduced pressure, and the crude product was load to flash column chromatography for purification (CH₂Cl₂/methanol 10:1), and (E)-3-((3-bromoallyl)oxy)-7-nitro-10H-phenothiazine (Compound SIL28) was obtained as a red solid (8.3 mg, 67% yield). 1H NMR (400 MHz, CDCl₃): δ 7.80-7.69 (m, 2H), 6.56-6.28 (m, 6H), 6.06-5.95 (br s, 1H), 4.38-4.28 (br s, 2H).

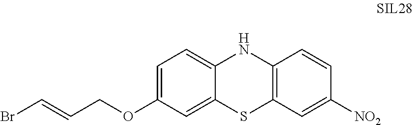 [SMILES: O=[N+]([O-])c1ccc2c(c1)Sc1cc(OC/C=C/Br)ccc1N2]

To a solution of (Z)-1-(3-((3-bromoallyl)oxy)-7-nitro-10H-phenothiazin-10-yl)ethanone (TZ17-16A, 28.0 mg) in acetonitrile (3 mL) was added excess DBU, and the resulting mixture was heated to 90° C. for 30 min. Solvents were evaporated, and the residue was purified by flash column chromatography (hexane/CH₂Cl₂1:1 to 1:2), and 3-nitro-7-(prop-2-yn-1-yloxy)-10H-phenothiazine (Compound TZ17-22) was obtained as a purple solid. 1H NMR (DMSO-d6): 3.58 (s, 1H), 4.71 (s, 2H), 6.62-6.67 (s, 4H), 7.73 (s, 1H), 7.84 (d, J=9.0 Hz, 1H), 9.43 (br s, 1H).

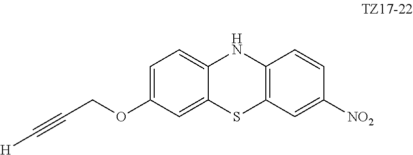 [SMILES: C#CCOc1ccc2c(c1)Sc1cc([N+](=O)[O-])ccc1N2]

To a mixture of (Z)-1-(3-((3-bromoallyl)oxy)-7-nitro-10H-phenothiazin-10-yl)ethanone (TZ17-16A, 8.0 mg) was added HCl (4M solution in methanol/H₂O 3:1). The resulting solution was refluxed for 3 hours. The flash was then cooled down to room temperature, and Na₂CO₃ (Sat.) was added to quench the reaction. The product was partitioned by CH₂Cl₂, dried. Solvents were removed by reduced pressure. The crude product was purified by flash column chromatography (hexane/CH₂Cl₂ 1:1), and the product was obtained (Z)-3-((3-bromoallyl)oxy)-7-nitro-10H-phenothiazine (Compound TZ17-24) as a purple solid (4.5 mg, 72% yield). ¹H NMR (400 MHz, CDCl₃): 7.80-7.62 (br s, 2H), 6.80-6.28 (m, 6H), 6.06-5.90 (br s, 1H), 4.68-4.40 (br s, 2H).

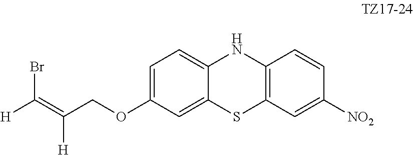 [SMILES: O=[N+]([O-])c1ccc2c(c1)Sc1cc(OC/C=C\Br)ccc1N2]

[¹²⁵I]SIL23 was radiosynthesized by a halogen exchange reaction under the catalysis of Cu⁺ from the corresponding bromo-substituted precursor (SIL28).

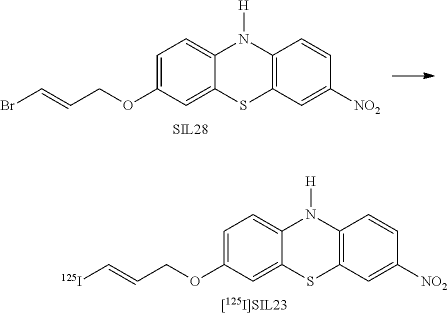 [SMILES: O=[N+]([O-])c1ccc2c(c1)Sc1cc(OC/C=C/Br)ccc1N2 ; O=[N+]([O-])c1ccc2c(c1)Sc1cc(OC/C=C/[125I])ccc1N2]

Two stock solutions were prepared for the radiolabelling: Solution A: ascorbic acid (116 mg) and SnSO₄ (6 mg) dissolved in water (1 ml); Solution B: CuSO₄.5H₂O (2 mg) and 98% H₂SO₄ (60 μl) dissolved in water (2 ml). Both solutions were flushed with helium for 30 min. The precursor (1 mg) was dissolved in DMSO (300 μl) in a 2 ml reaction vessel containing a stir bar. Solution A (100 μl) and Solution B (200 μl) were added into the reaction vessel under nitrogen protection. The reaction vessel was sealed immediately after the addition of [¹²⁵I]NaI (3 mCi) and heated at 130° C. for about 1 h. After the reaction mixture was cooled to room temperature, the reaction solution was diluted with 3 ml mobile phase and injected into a HPLC reverse phase semi-preparative column (Agilent SB-C18, 5 μm, 10×250 mm). The radioactive product was collected from 30 to 35 min on the HPLC condition (mobile phase: acetonitrile/water 60/40, v/v; flow rate: 4 ml/min; UV at 254 nm). The collected fraction was diluted with 40 ml of water and loaded on a C-18 Sep-Pak cartridge. After purification by HPLC again, the final product was eluted by ethanol (1 ml) to form the final solution (1.3 mCi, radiochemical yield 43%). Since [¹²⁵I]SIL23 is effectively separated from the precursor on the semipreparative HPLC system described above and carrier-free [¹²⁵I]NaI was used, it is assumed that the [¹²⁵I]SIL23 is carrier-free with a theoretical specific activity of 2200 Ci/mmol.

### Example 36

### Radioligand Binding Assays and Competitive Binding Assays with Human Postmortem Brain Tissue Preparations

Brain tissue samples were selected from an autopsy case series of patients evaluated for parkinsonism by movement disorders specialists at the Movement Disorders Center of Washington University School of Medicine in St. Louis. The clinical diagnosis of idiopathic PD was based on modified United Kingdom Parkinson's Disease Society Brain Bank clinical diagnostic criteria with clear clinical response to levodopa [6]. Dementia was determined by a movement disorders specialist based on clinical assessment of cognitive dysfunction sufficiently severe to impair activities of daily living, with further evaluation of cognitive impairment using the AD8 and Mini-Mental Status Exam (MMSE) [7, 8]. LB stage was assessed at autopsy using a PD staging scale (range: 0, 1-6) [9]. PD cases were selected based on a clinical diagnosis of PD plus dementia, Braak LB stage 5-6 pathology, and the absence of significant Aβ or tau pathology determined by immunohistochemistry. Control cases were selected based on the absence of α-synuclein, Aβ and tau pathology. Samples were used from both male and female subjects.

Transgenic mouse lines expressing human A53T α-synuclein (M83 line) or human wild type α-synuclein (M7 line) were obtained from the University of Pennsylvania. Mice used in this study were homozygous for each of the transgenes and were bred on mixed B6C3H and 129Sv backgrounds. M83 mice were observed for the development of neurological impairment and were euthanized after the onset of motor impairment. Brain tissue was removed and midbrain/pons/medulla tissue samples were dissected by first making a midsagittal cut using a brain matrix, which was then followed by an axial cut at the cervicomedullary junction and a second axial cut rostral to the superior colliculus for each hemisphere. Both male and female mice were used in the study.

### Preparation of recombinant α-synuclein and tau protein

Recombinant protein was produced in E. Coli using protocols based on previously described methods for α-synuclein [11-13] and tau [14]. BL21(DE3)RIL E. coli were transformed with a pRK172 bacterial expression plasmid containing the human α-synuclein coding sequence. Freshly transformed BL21 colonies were inoculated into 2 L baffled flasks containing 250 ml sterilized TB (1.2% bactotryptone, 2.4% yeast extract, 0.4% glycerol, 0.17 M KH₂PO₄, 0.72 M K₂HPO₄) with 50 μg/ml ampicillin, and incubated overnight at 37° C. with shaking Overnight cultures were centrifuged at 3,900×g for 10 min at 25° C. and the bacterial pellets were resuspended by gentle vortexing in 20 ml osmotic shock buffer (30 mM Tris-HCl, 2 mM EDTA, 40% Sucrose, pH 7.2) and then incubated at room temperature for 10 min. The cell suspension was then centrifuged at 8,000×g for 10 min at 25° C. and the pellet was resuspended in 22.5 ml cold H₂O before adding 9.4 μl 2 M MgCl₂ to each tube. The suspension was incubated on ice for 3 min prior to centrifugation at 20,000×g for 15 min at 4° C. After the supernatant was transferred to a fresh tube, streptomycin was added to a final concentration of 10 mg/ml and centrifuged at 20,000×g for 15 min at 4° C. The supernatant from this step was collected and dithiothreitol (DTT) and Tris-HCl pH 8.0 were added to final concentrations of 1 mM and 20 mM respectively, before boiling for 10 min to precipitate heat-sensitive proteins, which were pelleted at 20,000×g for 15 min at 4° C. The supernatant was collected and filtered through a 0.45 μm surfactant-free cellulose acetate filter (Corning) before loading onto a 1 ml DEAE Sepharose column equilibrated in 20 mM Tris-HCl pH 8.0, 1 mM EDTA, and 1 mM DTT. The DEAE column was washed with 20 mM Tris-HCl pH 8.0, 1 mM EDTA, 1 mM DTT before eluting α-synuclein protein in 20 mM Tris-HCl pH 8.0 buffer with 1 mM EDTA, 1 mM DTT and 0.3 M NaCl. Purified α-synuclein protein was dialyzed overnight in 10 mM Tris-HCl pH 7.6, 50 mM NaCl, 1 mM DTT. Preparations contained greater than 95% α-synuclein protein as determined by sodium dodecyl sulfate polyacrylamide gel electrophoresis (SDS-PAGE) and bicinchoninic acid (BCA) protein assay (Thermo Scientific, Rockford, Ill.), with a typical yield of 30 mg protein per 250 ml culture.

Recombinant tau protein was produced in E. Coli. BL21(DE3)RIL E. Coli were transformed with a pRK172 bacterial expression plasmid encoding a human tau fragment containing the four microtubule binding repeats (amino acids 243-375) [15]. Cultures were inoculated and grown overnight as above for α-synuclein protein production. Purified tau protein was prepared using the method described in reference [14] and dialyzed overnight in 100 mM sodium acetate pH 7.0.

### Preparation of Recombinant α-Synuclein Fibrils

Purified recombinant α-synuclein monomer (2 mg/ml) was incubated in 20 mM Tris-HCl, pH 8.0, 100 mM NaCl for 72 h at 37 C with shaking at 1000 rpm in an Eppendorf Thermomixer. To determine the concentration of fibrils, the fibril reaction mix was centrifuged at 15,000×g for 15 min to separate fibrils from monomer. The concentration of α-synuclein monomer in the supernatant was determined in a BCA protein assay according to the manufacturer's instructions, using a bovine serum albumin (BSA) standard curve. The measured decrease in α-synuclein monomer concentration was used to determine the concentration of fibrils in the 72 h fibril reaction mixture.

### Preparation of Aβ₁₋₄₂ Fibrils

Synthetic Aβ₁₋₄₂ peptide (1 mg) (Bachem, Torrance, Calif.) was first dissolved in 50 μl DMSO. An additional 925 μl of mQ-H2O was added. Finally, 25 μl 1M Tris-HCl pH 7.6 was added to bring the final peptide concentration to 222 μM (1 mg/ml) [16]. The dissolved peptide was incubated for 30 h at 37° C. with shaking at 1000 rpm in an Eppendorf Thermomixer. Fibril formation was confirmed by ThioT fluorescence. To determine the concentration of fibrils, the fibril reaction mix was centrifuged at 15,000×g for 15 min to separate fibrils from monomer. The concentration of Aβ monomer in the supernatant was determined in a BCA protein assay using a BSA standard curve that contained DMSO at a percentage equivalent to the samples.

### Preparation of Recombinant Tau Fibrils

Purified recombinant tau monomer (300 μg/ml) was incubated in 20 mM Tris-HCl pH 8.0, 100 mM NaCl, 25 μM low molecular weight heparin, 0.5 mM DTT for 48 h at 37° C. with shaking at 1000 rpm in an Eppendorf Thermomixer. To determine the concentration of fibrils, the fibril reaction mixer was centrifuged at 15,000×g for 15 min to separate fibrils from monomer. The concentration of tau monomer in the supernatant was determined in a BCA protein assay along with a BSA standard curve. The measured decrease in monomer concentration was used to determine the concentration of tau fibrils in the 48 h fibril reaction mixture.

### Preparation of α-Synuclein, Aβ₁₋₄₂, and Tau Fibrils for Binding and Competition Assays

The prepared fibril mixture was centrifuged at 15,000×g for 15 min to prepare fibrils for binding assays. The supernatant was discarded and the fibril pellet was resuspended in 30 mM Tris-HCl pH 7.4, 0.1% BSA to achieve the desired concentration of fibrils for use in the assay.

### Preparation of Human Brain Tissue for In Vitro Binding and Competition Studies

Grey matter was isolated from frozen postmortem frontal cortex tissue by dissection with a scalpel. To prepare insoluble fractions, dissected tissue was sequentially homogenized in four buffers (3 ml/g wet weight of tissue) with glass Dounce tissue grinders (Kimble): 1) High salt (HS) buffer: 50 mM Tris-HCl pH 7.5, 750 mM NaCl, 5 mM EDTA; 2) HS buffer with 1% Triton X-100; 3) HS buffer with 1% Triton X-100 and 1 M sucrose; and 4) phosphate buffered saline (PBS). Homogenates were centrifuged at 100,000×g after each homogenization step and the pellet was resuspended and homogenized in the next buffer in the sequence. For comparison in initial binding studies, crude tissue homogenates were also prepared by homogenization of tissue in only PBS.

### In Vitro Saturation Binding Studies of [¹²⁵I]SIL23

A fixed concentration (1 μM/well) of α-synuclein, Aβ, or tau fibrils were incubated for 2 h at 37° C. with increasing concentrations of [¹²⁵I]SIL23 (6.25-600 nM) in 30 mM Tris-HCl pH 7.4, 0.1% BSA in a reaction volume of 150 μl. A fixed ratio of hot:cold SIL23 was used for all radioligand concentrations. The exact hot:cold SIL23 ratio was measured in each experiment by counting a 10 μl sample of the radioligand preparation in a scintillation counter. Binding of [¹²⁵I]SIL23 to human brain homogenates was assessed by incubating 10 μg samples of insoluble fraction or 50 μg of crude brain tissue homogenate, from PD-dementia or control subjects, with increasing concentrations of [¹²⁵I]SIL23 (6.25-600 nM). Nonspecific binding was determined in a duplicate set of binding reactions containing the competitor ThioT. Bound and free radioligand were separated by vacuum filtration through 0.45 μm PVDF filters in 96-well filter plates (Millipore), followed by three 200 μl washes with cold assay buffer. Filters containing the bound ligand were mixed with 150 μl of Optiphase Supermix scintillation cocktail (PerkinElmer) and counted immediately. All data points were performed in triplicate. The dissociation constant (K_{d}) and the maximal number of binding sites (Bₘₐₓ) values were determined by fitting the data to the equation Y=Bₘₐₓ*X/(X+K_{d}) by nonlinear regression using Graphpad Prism software (version 4.0).

### In Vitro Competition Studies of [¹²⁵I]SIL23

Competition assays used a fixed concentration of fibrils (1 μM) or tissue (10 μg/150 μl reaction) and [¹²⁵I]SIL23 (200 nM, consisting of a ratio of 1:400 hot:cold SIL23) and varying concentration ranges of cold competitor, depending on the ligand. Competitors were diluted in 30 mM Tris-HCl pH 7.4, 0.1% BSA. Reactions were incubated at 37° C. for 2 h before quantifying bound radioligand as described above for the saturation binding assay. All data points were performed in triplicate. Data were analyzed using Graphpad Prism software (version 4.0) to obtain EC₅₀ values by fitting the data to the equation Y=bottom+(top-bottom)/(1+10^{(x-10gEC}^{₅₀}⁾). Kᵢ values were calculated from EC₅₀ values using the equation KᵢEC₅₀/(1+[radioligand]/K_{d}).

### Extraction of Insoluble α-Synuclein for Western Blot and ELISA

Insoluble α-synuclein was isolated by sequential extraction of frozen postmortem human brain tissue as described previously [17]. Grey matter was isolated from frozen postmortem frontal cortex tissue by dissection with a scalpel. To prepare insoluble fractions for Western blot and ELISA analysis, dissected tissue was sequentially extracted in six buffers (3 ml/g wet weight of tissue) with glass Dounce tissue grinders (Kimble) [17]: 1, 2) High salt (HS) buffer: 50 mM Tris-HCl pH 7.5, 750 mM NaCl, 5 mM EDTA; 3) HS buffer with 1% Triton X-100; 4) HS buffer with 1% Triton X-100 and 1M sucrose; and 5, 6) 1×radioimmunoprecipitation assay (RIPA) buffer. Extracts were centrifuged at 100,000×g after each step and the pellet was resuspended and extracted in the next buffer in the sequence. The final pellet was then resuspended in 50 mM Tris-HCl pH 8.0, 2% SDS (1 ml/g wet weight of tissue) and sonicated for 5 sec with 5 sec rest intervals in between for a total sonication time of 30 sec. Sonicated samples were centrifuged at 100,000×g and the supernatant was saved (SDS extract). The pellet was resuspended in 70% formic acid (1 ml/g wet weight of tissue) and sonicated for 5 sec with 5 sec rest intervals in between for a total sonication time of 30 sec. The formic acid was evaporated in a speed vacuum for 2 h. Then 1 volume of 50 mM Tris-HCl pH 8.0, 2% SDS was added to each sample to solubilize the protein. The samples were sonicated for 5 sec with 5 sec rest intervals in between for a total sonication time of 30 sec.

### Western Blot

Western Blot was performed as described previously [18]. Frontal cortex PD and control SDS extracts (8 μl) and anterior cingulate and temporal cortex PD extracts (4 μl) were run on an 18% Tris-glycine gel (Bio-Rad Criterion) and transferred to a nitrocellulose membrane as described previously [18]. The membrane was blocked with 5% nonfat milk in Tris buffered saline (TBS) with 0.1% Tween-20 for 1 h at room temperature, followed by incubation overnight at 4° C. with syn1 (BD Biosciences) or syn303 [19], both mouse monoclonal antibodies against α-synuclein. The blot was then incubated with HRP-conjugated anti-mouse secondary antibody for 1 h at room temperature, followed by washing and detection with Immobilon enhanced chemiluminescence (ECL) reagent (Millipore). The blot was imaged with the G:Box Chemi XT4 (Synpotics) imager and was quantified using Multi-Gauge software (Fujifilm). Western blots included a standard curve of recombinant α-synuclein protein ranging from 2.5 ng to 30 ng. The ECL signal was linear over the range of the standards.

### Sandwich ELISA for α-Synuclein

The levels of α-synuclein were measured by sandwich ELISA following the sequential extraction procedure. Mouse monoclonal α-synuclein 211 (Santa Cruz Biotechnology) was used as the capture antibody and biotinylated goat polyclonal anti-human synuclein-α (R&D Systems) was used as the detection antibody. PBS with 0.05% Tween 20, 2% BSA was used to block for 1 h at 37° C. before adding samples. All washes were done in PBS-Tween 20. Bound detection antibody was quantified using Streptavidin Poly HRP80 (Fitzgerald) and SuperSlow 3,3′,5,5′-Tetramethylbenzidine (TMB) liquid substrate (Sigma-Aldrich). The standard curve was generated by combining bacterial recombinant α-synuclein with extracts prepared from control tissue samples, and ranged from 0 ng/well to 100 ng/well.

### [¹²⁵I]SIL23 Binds to Recombinant α-Synuclein Fibrils

As demonstrated in Example 33, (3-iodoallyl)oxy-phenothiazine (SIL23), displayed moderate affinity for α-synuclein fibrils (K, approx. 60 nM) in the ThioT competition assay, and was suitable for radiolabeling with ¹²⁵I. Based on this result, [¹²⁵I]SIL23 was synthesized to characterize the binding properties of this radioligand in fibril and tissue assays, and to demonstrate its utility for screening additional compounds as candidate imaging ligands, which is an essential step for the development of other imaging agents for PD.

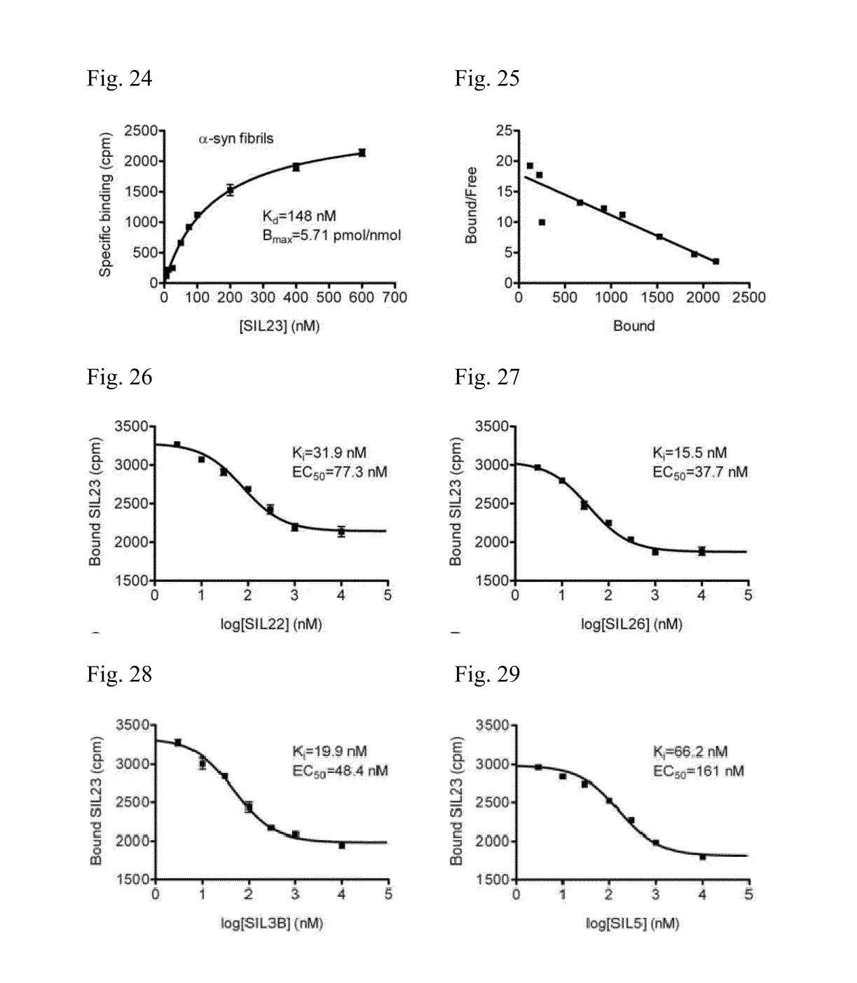

Methods were developed to measure the in vitro binding affinity of [¹²⁵I]SIL23 to recombinant α-synuclein fibrils in saturation binding experiments. Recombinant α-synuclein fibrils were incubated with increasing concentrations of [¹²⁵I]SIL23. Nonspecific binding was determined in parallel reactions containing ThioT, unlabeled SIL23, or the phenothiazine analogue SIL5 as competitors, or in reactions containing radioligand but no fibrils, all of which yielded similar specific binding values. The binding data were analyzed by curve fitting using nonlinear regression to obtain K_{d} and Bₘₐₓ values. Specific binding of [¹²⁵I]SIL23 to α-synuclein fibrils was observed with a K_{d} of 148 nM and a Bₘₐₓ of 5.71 pmol/nmol α-synuclein monomer. A representative plot of specific binding versus [¹²⁵I]SIL23 concentration is shown in FIG. 24. Consistent binding values were observed for five independently prepared fibril batches with K_{d} values ranging from 120 nM to 180 nM. Scatchard analysis, which is shown in FIG. 25 indicates that the binding fits a one-site model.

The [¹²⁵I]SIL23 competitive binding assay was developed to enable the evaluation of binding affinities for additional phenothiazine analogues. Fixed concentrations of α-synuclein fibrils and [¹²⁵I]SIL23 were incubated with increasing concentrations of each phenothiazine compound. Four analogues of [¹²⁵I]SIL23, compounds SIL22 (FIG. 26), SIL26 (FIG. 27), SIL3B (FIG. 28), and SIL5 (FIG. 29), were tested and had respective Kᵢ values of 31.9 nM, 15.5 nM, 19.9 nM, and 66.2 nM, all with significantly higher affinities than the K_{d} for SIL23, indicating that SIL23 binding assays can guide the optimization of compound structures to increase binding affinity for α-synuclein fibrils. The amount of bound radioligand is plotted on FIGS. 26-29 as a function of the concentration of unlabeled competitor ligand in the incubation mixture. Data points represent mean+/−s.d. (n=3). EC₅₀ values were determined by fitting the data to the equation Y=bottom+(top-bottom)/(1+10^{(x-logEC}^{₅₀}⁾).

### [¹²⁵I]SIL23 and Additional SIL Analogues Exhibit Higher Binding Affinity to Recombinant α-Synuclein Fibrils Compared to Synthetic Aβ₁₋₄₂ or Recombinant Tau Fibrils

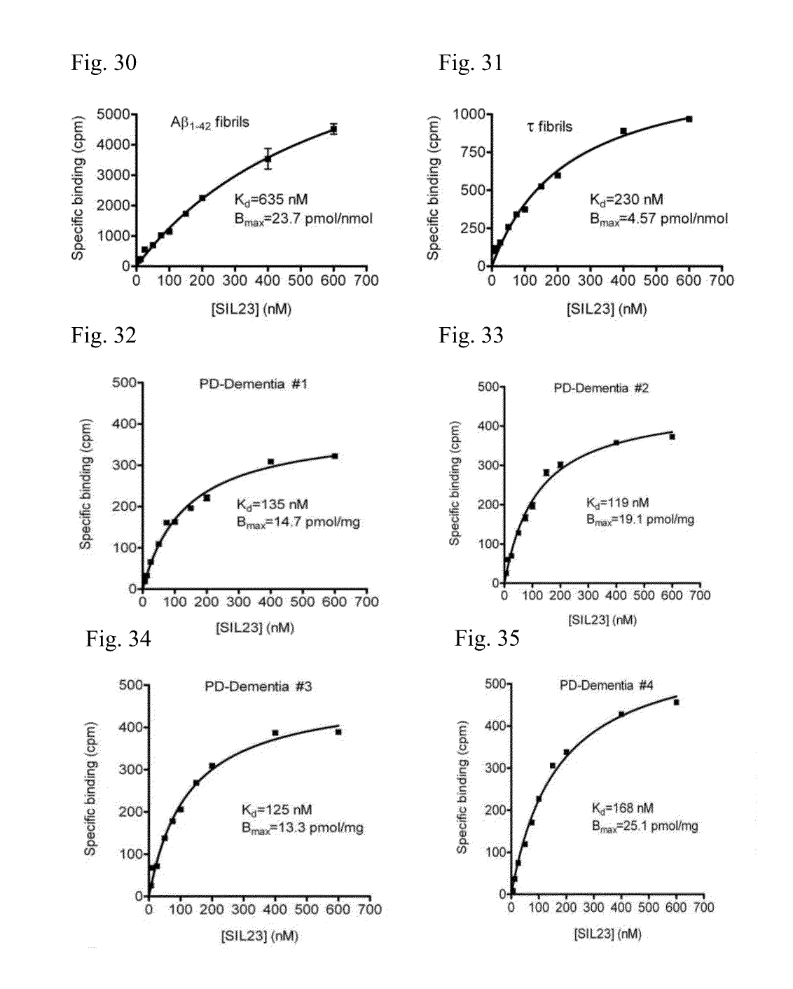

To determine the specificity of [¹²⁵I]SIL23 for recombinant α-synuclein fibrils, in vitro saturation binding studies were performed on synthetic Aβ₁₋₄₂ (FIG. 30) and recombinant tau fibrils (FIG. 31) and compared the results to data obtained from binding studies conducted on α-synuclein fibrils. Overall, the affinity of [¹²⁵I]SIL23 for Aβ₁₋₄₂ (K_{d} 635 nM, Bₘₐₓ 23.7 pmol/nmol) fibrils was 5-fold lower than that observed for α-synuclein fibrils. The affinity for tau fibrils (IQ 230 nM, Bₘₐₓ 4.57 pmol/nmol) was approximately 2-fold lower than α-synuclein fibrils.

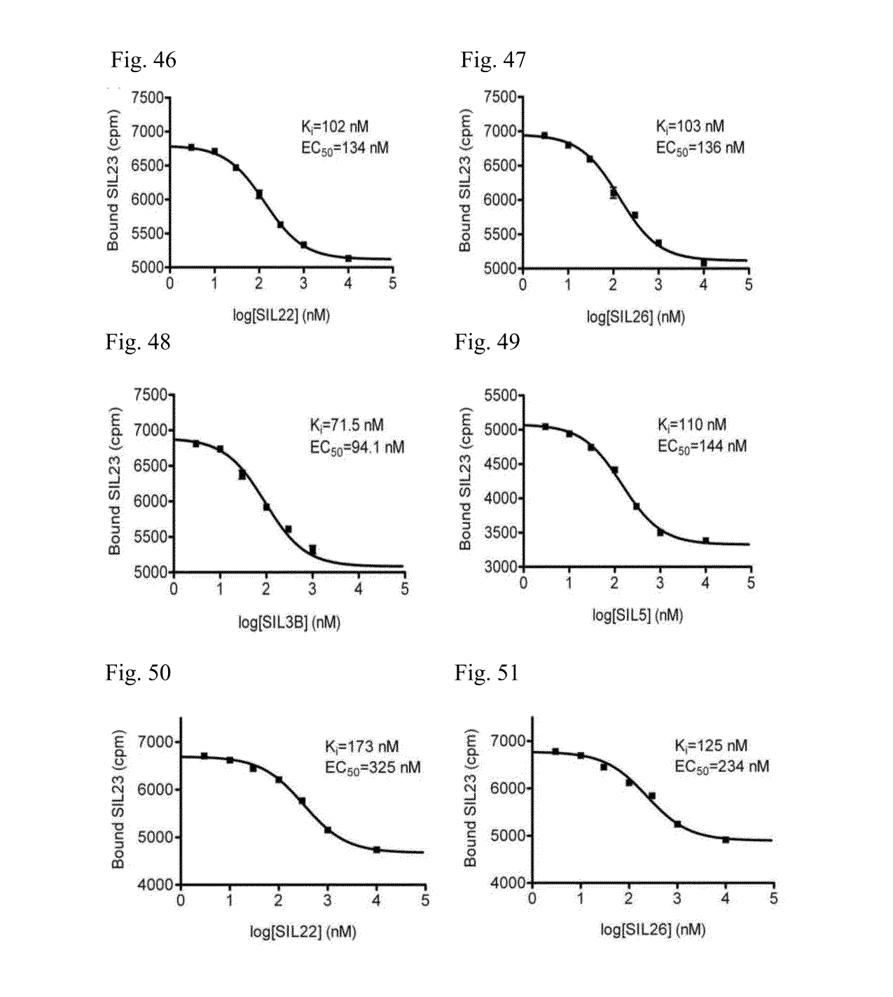

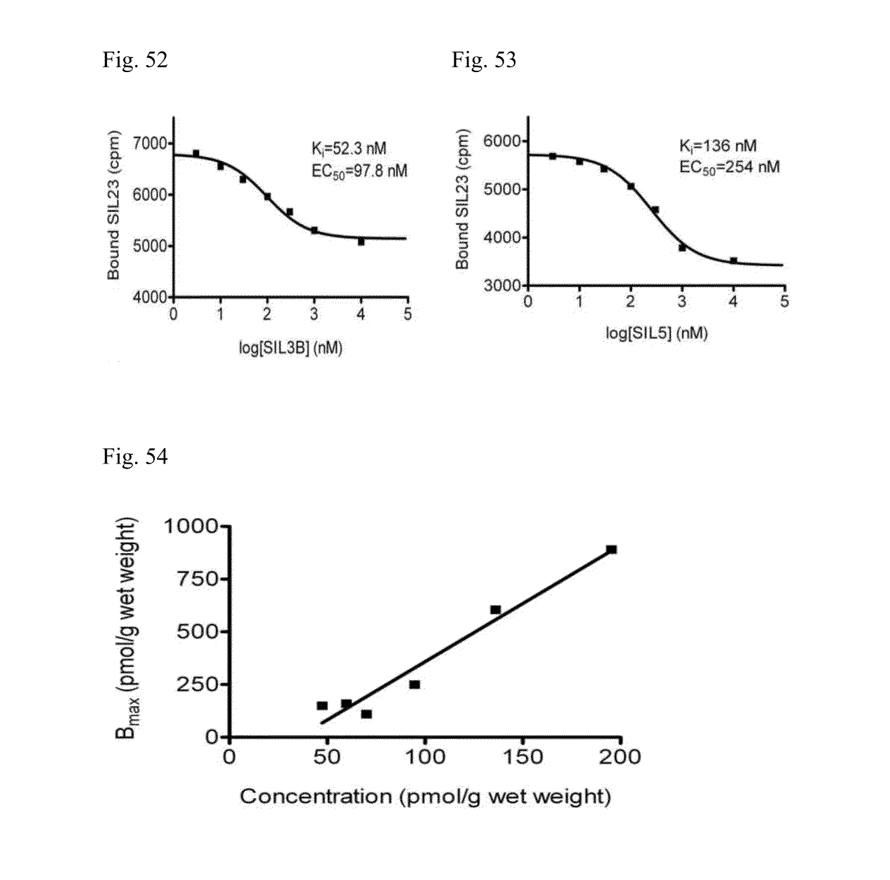

To determine the specificity of other phenothiazine analogues for α-synuclein fibrils, radioligand competition assays were performed with Aβ₁₋₄₂ (FIGS. 46-49) and tau fibrils (FIGS. 50-53) and compared the obtained Ic values to those obtained in radioligand competition assays with α-synuclein fibrils. All of the phenothiazine analogues (compounds SIL22, SIL26, SIL3B, and SIL5) examined in this study were selective for α-synuclein fibrils over Aβ₁₋₄₂ and tau fibrils, but selectivity varied among analogues. Table 2 provides a comparison of Kᵢ values for phenothiazine analogues in assays with α-synuclein, Aβ₁₋₄₂, and tau fibrils and illustrates relative selectivity for α-synuclein over Aβ₁₋₄₂ and tau. Kᵢ values were calculated from EC₅₀ values using the equation Kᵢ=EC₅₀/(1+[radioligand]/K_{d}). 95% confidence intervals for Kᵢ values are shown in parentheses. SIL26, which has the highest affinity for α-synuclein fibrils (Kᵢ 15.5 nM), has more than 6-fold lower affinity for Aβ₁₋₄₂ (Kᵢ 103 nM) and more than 7-fold lower affinity for tau fibrils (Kᵢ 125 nM). These results obtained in SIL23 assays with Aβ₁₋₄₂ and tau fibrils indicate that variations in phenothiazine structure can enhance selectivity as well as affinity for α-synuclein fibrils.

TABLE 2
Comparison of Kᵢ values for SIL analogues in assays
with α-synuclein, Aβ₁₋₄₂, and tau fibrils

| | α-synuclein | α-synuclein | α-synuclein |
|---|---|---|---|
| Phenothiazine | fibrils | fibrils | fibrils |
| analogue | Kᵢ (nM) | Kᵢ (nM) | Kᵢ (nM) |
| SIL22 | 31.9 (22.1-45.9) | 102 (87.3-119) | 173 (144-208) |
| SIL26 | 15.5 (11.7-20.6) | 103 (83.6-128) | 125 (97.7-160) |
| SIL3B | 19.9 (14.9-26.7) | 71.5 (54.9-93.2) | 52.3 (38.8-70.4) |
| SIL5 | 66.2 (49.2-89.1) | 110 (94.7-127) | 136 (112-165) |

### [¹²⁵I]SIL23 Binds to Human PD Brain Homogenates

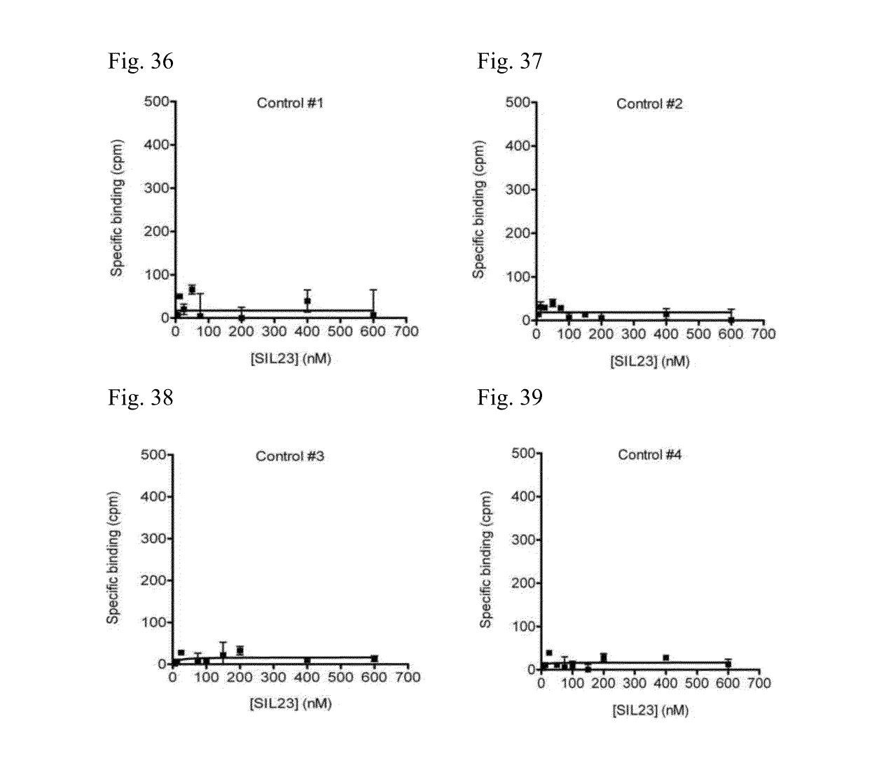

Previous studies have utilized binding assays with postmortem human brain homogenates to evaluate candidate amyloid imaging agents. A similar assay with PD tissue was developed to determine whether a binding site identified on recombinant α-synuclein fibrils is also present in PD tissue, and to determine whether the density of binding sites is high enough to image fibrillar α-synuclein in vivo. To evaluate [¹²⁵I]SIL23 binding to fibrillar α-synuclein in LBs and LNs present in PD brain, the in vitro binding of [¹²⁵I]SIL23 in postmortem brain tissue from PD was compared to control cases (Table 3), using insoluble fractions prepared from PD (n=4) and control (n=4) human brain tissue samples. K_{d} values for the PD cases ranged from 119 nM to 168 nM (Bₘₐₓ range 13.3-25.1 pmol/mg) (FIGS. 32-35). In contrast, no significant [¹²⁵I]SIL23 binding was detected in the samples from the control cases (FIGS. 36-39). These results indicate that [¹²⁵I]SIL23 binding affinity in PD brain samples is comparable to the binding affinity for recombinant α-synuclein fibrils.

TABLE 3
Clinical and demographic information for autopsy
cases utilized for binding studies.

| Case | | | Clinical | |
|---|---|---|---|---|
| number | Age | Gender | Diagnosis | Pathologic findings |
| PD 1 | 69 | M | PD, dementia | Diffuse Lewy body |
| | | | | disease |
| PD 2 | 82 | F | PD, dementia | Diffuse Lewy body |
| | | | | disease |
| PD 3 | 78 | M | PD, dementia | Diffuse Lewy body |
| | | | | disease |
| PD 4 | 77 | M | PD, dementia | Diffuse Lewy body |
| | | | | disease |
| PD 5 | 79 | M | PD, dementia | Diffuse Lewy body |
| | | | | disease |
| C1 | 74 | F | parkinsonism | Arteriosclerosis |
| C2 | 78 | F | parkinsonism, | Small vessel infarcts, |
| | | | dementia | argyrophilic grain |
| | | | | disease |
| C3 | 85 | M | parkinsonism | Argyrophilic grain |
| | | | | disease, |
| | | | | arteriosclerosis |
| C4 | 85 | M | parkinsonism, | Small and large vessel |
| | | | dementia | disease with neuronal |
| | | | | loss |

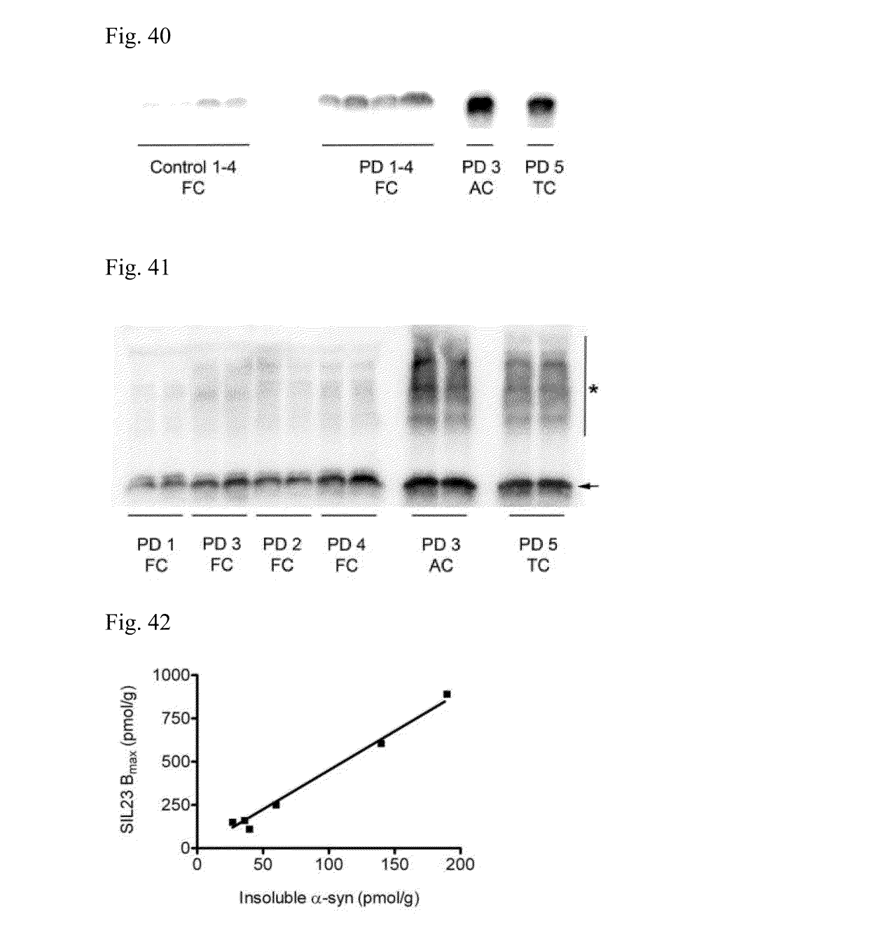

To determine whether SIL23 binding in different PD cases correlated with total levels of insoluble α-synuclein, western blots were performed on insoluble fractions prepared from PD (n=6) and control (n=4) human brain tissue samples. Western blot results shown in FIG. 40 indicate that PD cases had different levels of insoluble α-synuclein, with anterior cingulate and temporal cortex PD samples showing the highest levels. In contrast, control cases had very low levels of detectable α-synuclein in insoluble fractions, which could represent low-level carryover of soluble α-synuclein during sequential extraction. In addition to monomeric α-synuclein, higher molecular weight species, likely representing multimeric α-synuclein, were also observed on western blots of insoluble fractions from PD cases as shown in FIG. 41. Insignificant levels of α-synuclein were observed by western blot analysis of formic acid extracts from the sequential extraction procedure.

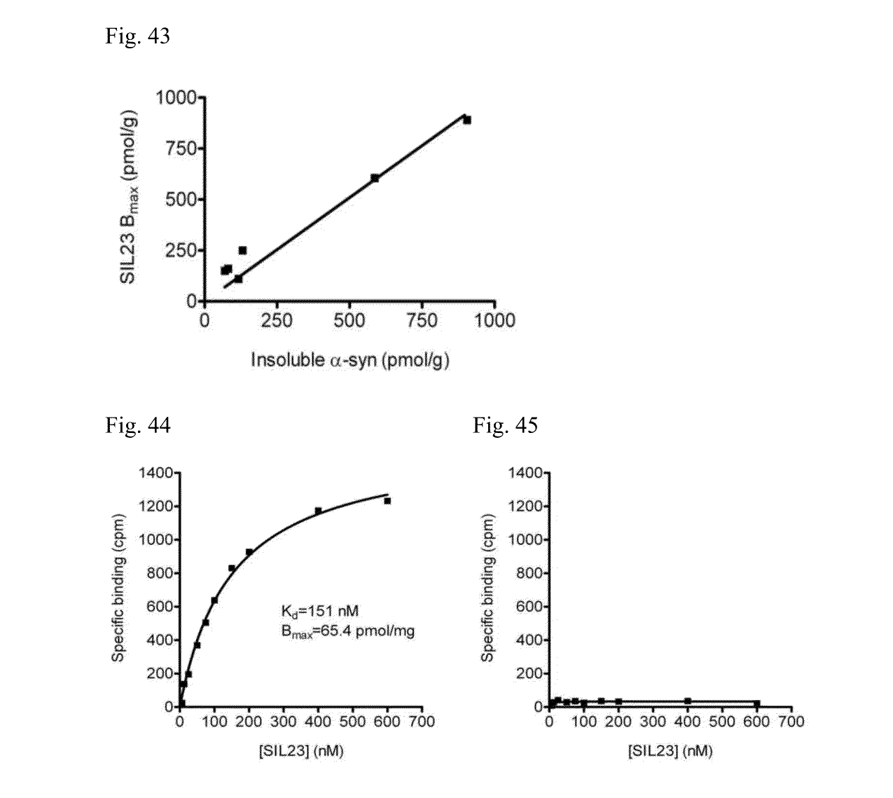

Total monomeric α-synuclein present in insoluble SDS fractions quantified from western blot correlated with Bₘₐₓ values measured by the radioligand binding assay (Pearson correlation coefficient R=0.99, p=0.0001) (as shown in FIG. 42). Bₘₐₓ values also correlated with insoluble α-synuclein measured by a sandwich ELISA (Pearson correlation coefficient R=0.98, p=0.0008) (as shown in FIG. 54). The ratios of Bₘₐₓ values to insoluble α-synuclein were approximately 4:1. If multimeric species were included in the quantification of insoluble α-synuclein, the ratios of Bₘₐₓ values to insoluble α-synuclein were approximately 1:1 (as shown on FIG. 43) and were also correlated (Pearson correlation coefficient R=0.99, p<0.001). Accuracies of these ratios may be limited by underestimation of insoluble α-synuclein due to low recovery during sequential extraction or incomplete solubilization of fibrils. A ratio of approximately 1 PiB binding site per 2 Aβ molecules has been observed in AD brain tissue [20].

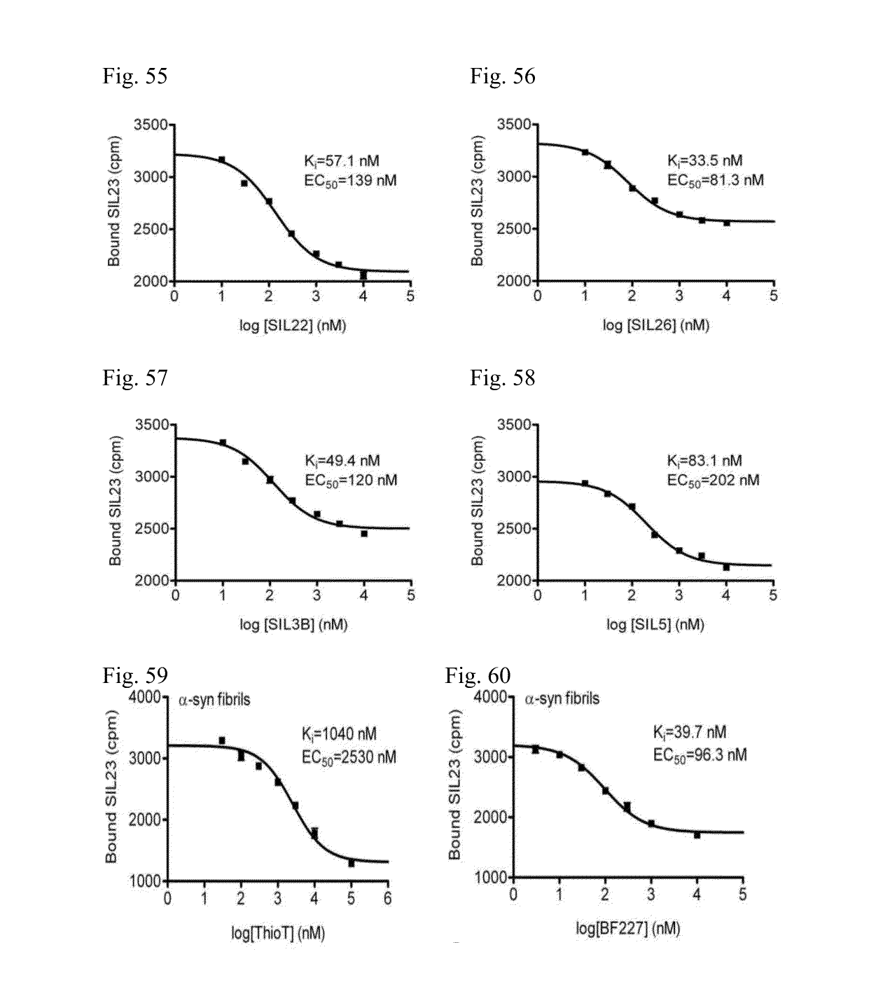

In vitro [¹²⁵I]SIL23 competition assays were used to evaluate the binding affinity of other SIL analogues with PD brain tissue samples (as shown in FIGS. 55-58). Fixed concentrations of homogenate and [¹²⁵I]SIL23 were incubated with increasing concentrations of unlabeled competitor ligands. The Kᵢ values obtained in assays with PD brain tissue homogenates were comparable overall to Kᵢ values obtained with α-synuclein fibrils but were approximately 2-fold lower for some ligands, indicating that SIL23 binding assays with recombinant α-synuclein fibril preparations accurately predict binding in tissue. Table 4 provides a comparison of Kᵢ values for SIL analogues in assays with recombinant α-synuclein fibrils versus human PD tissue. Kᵢ values were calculated from EC₅₀ values using the equation Kᵢ=EC₅₀/(1+[radioligand]/K_{d}). 95% confidence intervals for Kᵢ values are shown in parentheses.

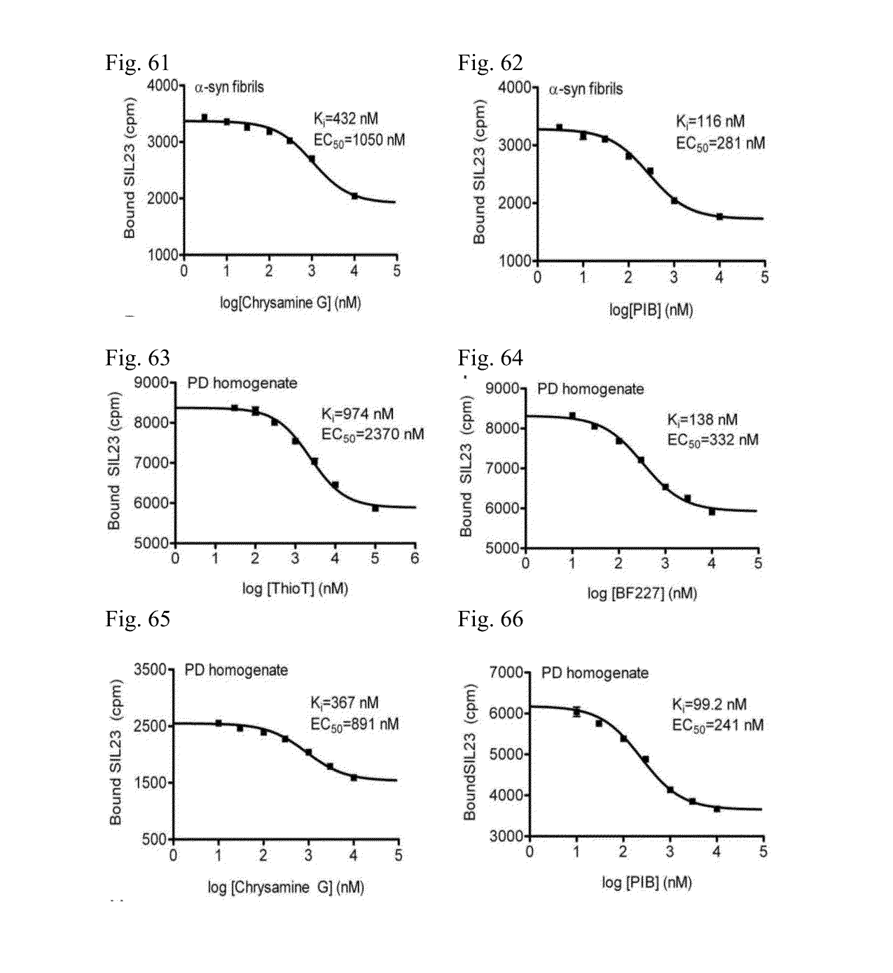

TABLE 4
Comparison of Kᵢ values for SIL analogues in assays with
recombinant α-synuclein and human PD brain homogenate

| | | Human PD brain |
|---|---|---|
| Phenothiazine | α-synuclein fibrils | homogenate |
| Analogue | Kᵢ (nM) | Kᵢ (nM) |
| SIL22 | 31.9 (22.1-45.9) | 57.1 (44.9-72.6) |
| SIL26 | 15.5 (11.7-20.6) | 33.5 (26.5-42.3) |
| SIL3B | 19.9 (14.9-26.7) | 49.4 (37.6-65.0) |
| SIL5 | 66.2 (49.2-89.1) | 83.1 (64.3-108) |

[¹²⁵I]SIL23 was also used as competition assay to evaluate other compounds known to bind amyloid fibrils. Fixed concentrations of α-synuclein fibrils and [¹²⁵I]SIL23 were incubated with increasing concentrations of PiB, ThioT, BF227, and Chrysamine G. Table 5 shows a comparison of Kᵢ values of previously reported ligands for α-synuclein fibrils determined in [¹²⁵I]SIL23 competitive binding assays with recombinant α-synuclein fibrils and PD tissue. Kᵢ values were calculated from EC₅₀ values using the equation Kᵢ=EC₅₀/(1+[radioligand]/K_{d}). 95% confidence intervals for Kᵢ values are shown in parentheses. See FIGS. 59-62 for plots of this binding data. ThioT displayed a Kᵢ of 1040 nM, which is comparable to the K_{d} measured for saturation binding of ThioT to α-synuclein fibrils [13]. Kᵢ values for PiB, BF227, and Chrysamine G were 116 nM, 39.7 nM, and 432 nM respectively. Additionally, [¹²⁵I]SIL23 competition assay in PD tissue homogenates was used to evaluate the binding properties of these compounds. The results are shown on Table 5 and FIGS. 63-66. Comparable Kᵢ values were obtained to those for recombinant α-synuclein fibrils, with the exception of BF227, which displayed weaker competition in PD tissue assays, possibly corresponding to previous observations that radiolabeled BF227 binding is not detectable in PD tissue [21]. The Kᵢ values for PiB and BF227 in the competition assays with α-synuclein fibrils were significantly higher than K_{d} values reported for the binding of radiolabeled PiB and BF227 to α-synuclein fibrils [21-23]. This could reflect differences in α-synuclein fibril preparations or may indicate that SIL23 binding sites only partially overlap with these previously reported ligands.

TABLE 5
Comparison of Kᵢ values of previously reported ligands for α-synuclein
fibrils determined in [¹²⁵I]SIL23 competitive binding assays
with recombinant α-synuclein fibrils and PD tissue

| | | Human PD brain |
|---|---|---|
| | α-synuclein fibrils | homogenate |
| Competitor | Kᵢ (nM) | Kᵢ (nM) |
| PIB | 116 (88.0-152) | 99.2 (72.4-136) |
| BF227 | 39.7 (28.1-55.9) | 138 (98.7-193) |
| Chrysamine G | 432 (325-573) | 367 (275-490) |
| Thioflavin T | 1040 (755-1440) | 974 (769-1230) |

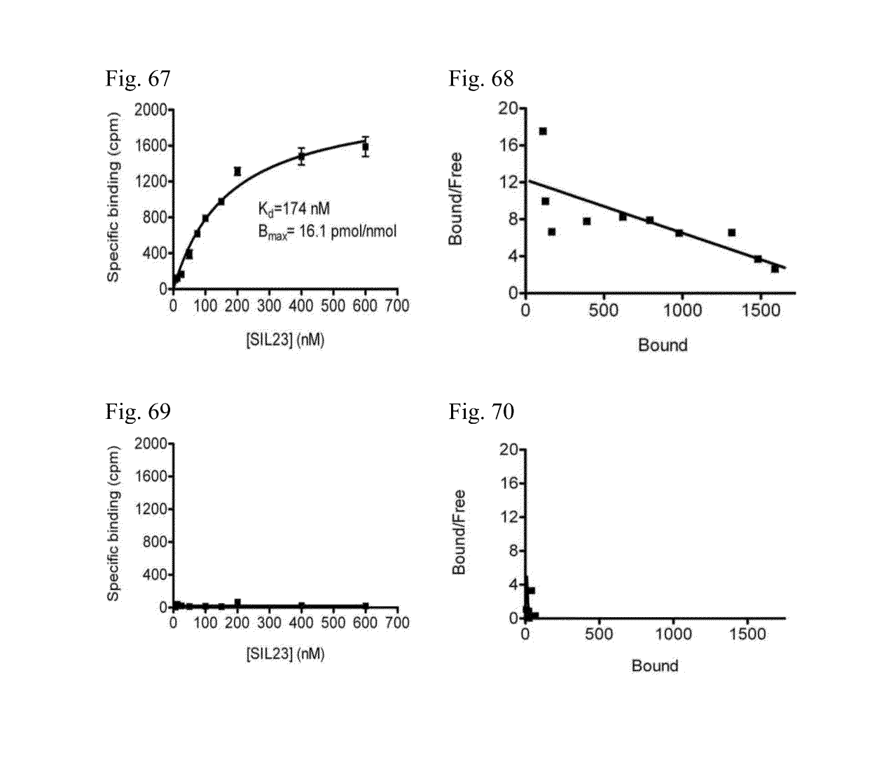

Binding site densities were evaluated in PD brain using saturation binding assays with insoluble fractions from other cortical regions as well as binding assays performed with unfractionated homogenates of brain tissue samples. Bₘₐₓ values for insoluble fractions were significantly higher in temporal cortex and anterior cingulate cortex compared to frontal cortex. Table 6 presents these results. For comparison to a previously reported average Bₘₐₓ value of 1407 pmol/g wet weight for PiB binding in AD brain [20], Bₘₐₓ values per gram wet weight were estimated for SIL23 binding in PD brain, which ranged from 8% to 63% of PiB values in AD. Saturation binding studies using crude brain tissue homogenates rather than insoluble protein preparations also yielded a similar K_{d} value of 174 nM for a PD case while no significant binding was observed for a control case. FIGS. 67-70 present the results of this study. The Bₘₐₓ value for the crude homogenate of a frontal cortex sample (PD 2) was 16.1 pmol/mg protein, which can be compared to an average Bₘₐₓ value of 8.8 pmol/mg protein observed in a similar assay for AV-45 binding in AD brain [24]. Nonspecific binding was significantly higher in binding assays with crude homogenates of brain tissue, possibly due to the high lipophilicity of SIL23 (calculated log P=5.7).

TABLE 6
Bₘₐₓ values determined in saturation binding
studies with PD brain tissue samples

| | | Bₘₐₓ (pmol/mg | Bₘₐₓ (pmol/g wet |
|---|---|---|---|
| PD case | Cortical region | insoluble protein) | weight) |
| 1 | midfrontal | 14.7 (13.9-15.5) | 145 |
| 2 | midfrontal | 19.1 (18.1-20.1) | 160 |
| 3 | midfrontal | 13.3 (12.7-13.9) | 108 |
| 4 | midfrontal | 25.1 (23.9-26.3) | 251 |
| 5 | temporal | 53.1 (45.7-60.5) | 607 |
| 3 | anterior cingulate | 91.1 (87.9-94.3) | 895 |

### [¹²⁵I]SIL23 Binding in a Transgenic Mouse Model for PD.

To determine whether SIL23 binding sites are also present in a transgenic mouse model for PD, brain tissue homogenates was prepared from transgenic mice expressing either a WT human α-synuclein transgene (M7 line) or a human α-synuclein transgene containing the A53T mutation that causes hereditary PD (M83 line) [10]. Accumulation of aggregated α-synuclein occurs primarily in brainstem and spinal cord of the M83 line but does not occur in the M7 line. M83 transgenic mice were observed and sacrificed when they displayed significant neurological impairment, which in this mouse line corresponds to the presence of aggregated α-synuclein in brain tissue. Tissue samples containing the midbrain, pons and medulla regions were dissected and processed by sequential extraction and centrifugation to prepare insoluble fractions. In saturation binding experiments, specific binding of [¹²⁵I]SIL23 in M83 tissue was observed with a K_{d} of 151 nM and Bₘₐₓ of 65.4 pmol/mg. FIG. 44 presents the results of the saturation binding experiments. In contrast, no significant [¹²⁵I]SIL23 binding was detected in M7 mouse brain homogenates. FIG. 45 presents the results of this binding experiment. The K_{d} for binding in M83 tissue is similar to that observed for both recombinant α-synuclein fibrils and human brain tissue. The Bₘₐₓ value is comparable to Bₘₐₓ values observed for human cortex from PD cases. These results indicate that this A53T α-synuclein transgenic mouse model is useful for evaluating in vivo binding of candidate α-synuclein imaging ligands, using micro-PET imaging or ex vivo autoradiography following radioligand injection.

The results establish the presence of a [¹²⁵I]SIL23 binding site on α-synuclein fibrils and define the binding properties of SIL23 in PD brain tissue. SIL23 binds with moderate affinity (K_{d} 148 nM) to α-synuclein fibrils. Furthermore, binding studies demonstrate that the fibrillar α-synuclein binding site is present in postmortem brain tissue from PD but not control cases and that binding site densities in PD tissue are comparable to binding site densities of Aβ imaging ligands in AD tissue. Competitive binding studies with [¹²⁵I]SIL23 enable phenothiazine analogues as well as other compounds to be screened for affinity and selectivity for fibrillar α-synuclein.

Accurate quantification of fibrillar α-synuclein in vivo requires a radiotracer with suitable affinity, selectivity, brain uptake and metabolism properties. Binding site density in brain is a critical factor in determining sensitivity and specificity for a radiotracer. This example shows binding site densities of 110-890 pmol/g wet weight, and 16 pmol/mg protein in binding assays with crude homogenates, for SIL23 binding in human postmortem cortex. These values are comparable to binding site densities observed for Aβ ligands in AD brain. The average PiB binding site density is 1407 pmol/g wet weight in AD brain, and the average AV-45 binding site density is 8.8 pmol/mg protein in AD brain. Based on K_{d} values of 2.5 nM for PiB and 3.7 nM for AV-45 binding to Aβ plaques, an α-synuclein binding site density of 16 pmol/mg protein, and an estimated brain concentration of 1 nM, a ligand that binds to the SIL23 binding site with a K_{d} of 7.3 nM could achieve binding to fibrillar α-synuclein in PD brain that is comparable to AV-45 binding in AD. Alternatively, a K_{d} of 2.5 nM, comparable to the K_{d} for PiB, will result in binding levels in cortex ranging from 8% to 63% of PiB levels.

### Example 37

### Radiosynthesis of [¹¹C]SIL5 and [¹⁸F]SIL26

All reagents and chemicals were purchased from Sigma-Aldrich Corporation (Milwaukee, Wis.) and used without further purification unless otherwise stated. The melting points of all intermediates and final compounds were determined on Hake-Buchler melting point apparatus and are uncorrected. ¹H and ¹³C NMR spectra were recorded on Varian-400 MHz. Spectra are referenced to the deuterium lock frequency of the spectrometer. The chemical shifts (in ppm) of residual solvents were found to be at 7.26 for CHCl₃ and at 2.50 for DMSO.

Compound 15, the precursor for radiolabeling [¹¹C]SIL5 and [¹⁸F]SIL26 was synthesized according to the Scheme shown in FIG. 2 and reported in Examples 3, 10, and 15 above with necessary modification as described below.

Acetyl chloride (360 mg, 4.59 mmol) was added into the solution of compound SIL5 (420 mg, 1.53 mmol) in dichloromethane (10 mL). The reaction mixture was stirred overnight at ambient temperature. The solvent and excess acetyl chloride were removed under vacuum. The residue was dissolved into ethyl acetate and washed with water and saturated sodium chloride solution. The organic extract was dried over anhydrous Na₂SO₄ and purified on silica gel column chromatography using ethyl acetate/hexane (1/2, v/v) as mobile phase to yield compound 14a (1-(3-methoxy-7-nitro-10H-phenothiazin-10-yl)ethanone) as yellow solid (267 mg, 55%). ¹H NMR (CDCl₃): δ 2.23 (s, 3H), 3.83 (s, 3H), 6.90 (d, J=9.0 Hz, 1H), 6.98 (s, 1H), 7.32 (d, J=8.7 Hz, 1H), 7.72 (d, J=8.7 Hz, 1H), 8.18 (d, J=8.7 Hz, 1H), 8.29 (s, 1H). ¹³C NMR (CDCl₃): δ 22.9, 55.7, 112.7, 114.0, 122.0, 122.9, 127.4, 127.9, 130.7, 133.2, 134.3, 144.7, 145.6, 158.5, 169.2. Anal. Calcd for C₁₅H₁₂N₂O₄S: C, 56.95; H, 3.82; N, 8.86. Found: C, 56.72; H, 3.89; N, 8.70. mp 155.9-156.8° C.

The solution of BBr₃ in dichloromethane (1.0 M, 4.2 mL) was added dropwise into the solution of compound 14a (267.3 mg, 0.84 mmol) in dichloromethane (15 mL) at −78° C. The reaction solution was stirred overnight at ambient temperature. The solvent was removed under vacuum. The residue was partitioned between ethyl acetate and water. The organic extract was dried over anhydrous Na₂SO₄ and purified on silica gel column chromatography using ethyl acetate/CH₂Cl₂ (1/10, v/v) as mobile phase to yield compound 15 (1-(3-Hydroxy-7-nitro-10H-phenothiazin-10-yl)ethanone) as yellow solid (207.4 mg, 81%). ¹H NMR (DMSO-d6): δ 2.15 (s, 3H), 6.82 (d, J=9.0 Hz, 1H), 6.93 (s, 1H), 7.47 (d, J=9.0 Hz, 1H), 7.82 (d, J=9.0 Hz, 1H), 8.22 (d, J=9.0 Hz, 1H), 8.39 (s, 1H), 10.00 (br s, 1H). ¹³C NMR (DMSO-d6): δ 23.0, 114.3, 115.3, 122.6, 123.2, 128.4, 128.5, 129.3, 132.3, 134.2, 145.1, 145.6, 156.7, 169.1. HRMS (ESI) m/z Calcd for C₁₄H₁₀N₂O₄S [M+1] 303.0440. Found: 303.0435. Purity: 98% (HPLC confirmed). mp 202.3-205.1° C.

To prepare [¹¹C]SIL5, [¹¹C]CH₃I was first produced from [¹¹C]CO₂ using a GE PETtrace MeI Microlab. Up to 1400 mCi of [¹¹C]CO₂ was produced from Washington University's JSW BC-16/8 cyclotron by irradiating a gas target of 0.2% O₂ in N₂ for 15-30 min with a 40 μA beam of 16 MeV protons. The GE PETtrace MeI microlab coverts the [¹¹C]CO₂ to [¹¹C]CH₄ using a nickel catalyst (Shimalite-Ni, Shimadzu, Japan P.N.221-27719) in the presence of hydrogen gas at 360° C.; it was further converted to [¹¹C]CH₃I by reacting with iodine that was held in a column in the gas phase at 690° C. Approximately 12 min after EOB, several hundred millicuries of [¹¹C]CH₃I was delivered as a gas to the hot cell where the radiosynthesis was accomplished.

[¹¹C]SIL5 was prepared according to the following reaction and as described below.

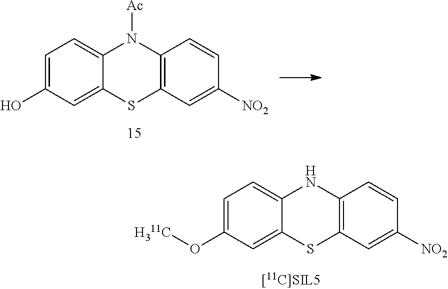 [SMILES: CC(=O)N1c2ccc(O)cc2Sc2cc([N+](=O)[O-])ccc21 ; [11CH3]Oc1ccc2c(c1)Sc1cc([N+](=O)[O-])ccc1N2]

Approximately 1.2 mg precursor 15 was placed in the reaction vessel and 0.20 mL of DMF was added, followed by 3.0 μL of 5 M NaOH aqueous solution. The mixture was thoroughly mixed on a vortex for 30 seconds. A stream of [¹¹C]CH₃I in helium was bubbled for 3 min into the reaction vessel. The sealed vessel was heated at 90° C. for 5 min, at which point the vessel was removed from heating. 20 μL 1,8-Diazabicyclo[5.4.0]undec-7-ene (DBU) in 50 μL DMF was added via syringe. The reaction mixture was heated at 90° C. for 7 min (Scheme 2), and quenched the reaction by adding 1.7 mL of HPLC mobile phase which was composed of acetonitrile/0.1 M ammonium formate buffer (60:40, v/v) and pH=˜4.5. The diluted solution was injected to a high performance liquid chromatography (HPLC) Phenomenex Luna C18 reverse phase column (9.4×250 mm); the product was eluted from the HPLC column using a flow rate of 4.0 mL/min and the UV wavelength as 254 nM. Under these conditions, the retention time of the precursor compound 15 was approximately 7 min; the retention time of [¹¹C]SIL5 was approximately 16 min. The [¹¹C]SIL5 product was collected into a vial containing 50 mL milli-Q water and passed through a Sep-Pak Plus C-18 cartridge (Waters, Milford, Mass., USA), in which the product was trapped. The trapped product was eluted with ethanol (0.6 mL) followed by 5.4 mL of 0.9% saline. After sterile filtration into a dose vial, the final product was ready for quality control (QC) analysis and animal studies. For the QC, the HPLC was performed on Phenomenex Prodigy C-18 reverse phase analytic HPLC column (250 mm×4.6 mm, 5 μA) and UV detection at 254 nm wavelength. The mobile phase was acetonitrile/0.1M ammonium formate buffer (80:20, v/v) using 1.5 mL/min flow rate. Under these conditions the retention time of [¹¹C]SIL5 was 4.82 min. The radioactive dose sample was authenticated by co-injection with the cold standard compound SIL5. The radiochemical purity was >99%, the chemical purity was >95%, the labeling yield was 35-45% (n=4, decay corrected to EOB) and the specific activity at time of delivery was >1500 mCi/gmol.

To prepare [¹⁸F]SIL26, [¹⁸F]fluoride was first produced in by ¹⁸O (p, n) ¹⁸F reaction through proton irradiation of enriched ¹⁸O water (95%) using a RDS-111 cyclotron (Siemens/CTI Molecular Imaging, Knoxyille, Tenn.). [¹⁸F]Fluoride is firstly passed through an ion-exchange resin and then is eluted with 0.02 M potassium carbonate (K₂CO₃) solution.

A 2-[¹⁸F]fluoroethyl tosylate reagent was prepared from 1,2-ethylene ditosylate according to the following reaction.

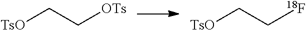 [SMILES: Cc1ccc(S(=O)(=O)OCCOS(=O)(=O)c2ccc(C)cc2)cc1 ; Cc1ccc(S(=O)(=O)OCC[18F])cc1]

A sample of approximately 150 mCi [¹⁸F]/fluoride was added to a reaction vessel containing Kryptofix [222] (6.5-7.0 mg). The syringe was washed with 2×0.4 mL ethanol. The resulting solution was evaporated under nitrogen flow with a bath temperature of 110° C. To the mixture, acetonitrile (3×1.0 mL) was added and water was azeotropically removed by evaporation. After all the water was removed, 5.0-5.5 mg of the corresponding precursor 1,2-ethylene ditosylate was dissolved in acetonitrile (200 μL) under vortex, and the precursor solution was transferred into the reaction vessel containing [¹⁸F]fluoride/Kryptofix/K₂CO₃. The reaction vessel was capped and the reaction mixture was briefly mixed, and then subjected to heating in an oil bath that was preheated to 110° C. for 10 min (Scheme 2).

After heating for 10 min, the reaction mixture was diluted with 3.0 mL of HPLC mobile phase (50:50 Acetonitrile/0.1M ammonium formate buffer, pH=˜6.5) and passed through an alumina neutral Sep-Pak Plus cartridge. The crude product was then loaded onto an Agilent SB-C18 semi-preparative HPLC column (250 mm×10 mm) with a UV detector set at 254 nm. The HPLC system used a 5 mL injection loop. At 4.0 mL/min flow rate, the retention time of the product was 9.5-10 min. The retention time of the precursor was 23-24 min. The radioactivity peak observed on HPLC was collected and diluted with 50 mL sterile water and the diluted collection went through a C-18 Sep-Pak Plus cartridge to trap the 2-[¹⁸F]fluoroethyl tosylate on the Sep-Pak. The trapped product was eluted with diethyl ether (2.5 mL).

[¹¹F]SIL26 was prepared according to the following reaction and as described below.

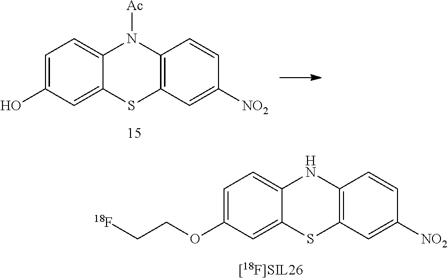 [SMILES: CC(=O)N1c2ccc(O)cc2Sc2cc([N+](=O)[O-])ccc21 ; O=[N+]([O-])c1ccc2c(c1)Sc1cc(OCC[18F])ccc1N2]

The eluted solution formed two phases, the top ethereal phase was transferred out, and the bottom aqueous phase was extracted with another 1 mL of ether. The combined ether extracts were passed through a set of two sodium sulfate Sep-Pak Plus dry cartridges into a reaction vessel. After ether was evaporated with a nitrogen stream at 25° C., 1.0 mg of precursor compound 15 was dissolved in 200 μL DMSO and was transferred to a vial containing 1-2 mg Cs₂CO₃. After vortexing for 1 min, the Cs₂CO₃ saturated solution was added into the reaction vessel containing the dried activity. The tube was capped and briefly swirled with a vortex, and then kept at 90° C. for 15 min. 10 μL 1,8-Diazabicyclo[5.4.0]undec-7-ene (DBU) in 50 μL DMSO was added via syringe. The reaction mixture was heated at 90° C. for 15 min. Subsequently, the residual mixture was diluted with 3 mL HPLC mobile phase (50:50 acetonitrile/0.1M formate buffer, pH=˜4.5) and loaded onto a Semi-Prep HPLC system for purification. The HPLC system contains a 5 mL injection loop, an Agilent SB C-18 column, a UV detector at 274 nm and a radioactivity detector. At 4.0 mL/min flow rate, the retention time of the product was 19-21 min, whereas the retention time of the precursor was 8-9 min. After the HPLC collection, and dilution with 50 mL sterile water, the product was trapped on a C-18 Sep-Pak Plus cartridge. The product was eluted with ethanol (0.6 mL) followed by 5.4 mL of 0.9% saline. After sterile filtration into a glass vial, the final product was ready for quality control (QC) analysis and animal studies. An aliquot of sample was assayed by an analytical HPLC system (Grace Altima C-18 column, 250×4.6 mm), UV at 274 nm; mobile phase consists of acetonitrile/0.1M, ammonium formate buffer (71/29, v/v), pH=˜4.5. Under these conditions, the retention time for [¹⁸F]SIL26 was approximately 4.86 min at a flow rate of 1.5 mL/min. The sample was authenticated by co-injecting with the cold standard SIL26 solution. The radiochemical purity was >98%, the chemical purity was >95%, the labeling yield was 55-65% (n=4, decay corrected) and the specific activity was >2000 mCi/gmol (decay corrected to end of synthesis, n=4).

The target compounds SIL5 and SIL26 possess a methyl or fluoroethoxyl group on the oxygen, therefore the radiosyntheses could be easily accessed by O-alkylation of the corresponding phenol precursor. However, to avoid undesired N-alkylation product, the acetyl protected precursor 15 was used as the precursor for the radiosyntheses. The synthesis of compound 15 was accomplished by a two-step strategy starting from SIL5 following the previously described procedure. N-acetylation of SIL5 was achieved using acetyl chloride. Removing the O-methyl group of 14a with boron tribromide afforded the phenol precursor 15, which was used in the radiosyntheses of SIL5 and SIL26. Due to the reaction scale difference, the yields for certain reactions have slightly different to our previous report.

The radiosynthesis of [¹¹C]SIL5 was accomplished by employing a two-step approach. The reaction of the phenol precursor 15 with [¹¹C]CH₃I was performed in DMF in the presence of NaOH, and the N-acetyl group on the C-11 labeled intermediate was removed by DBU following the literature procedure, [25] as described above. [¹¹C]SIL5 was obtained in approximately 35-45% overall radiochemical yield (RCY) after HPLC purification (n=4). The radiochemical purity of [¹¹C]SIL5 was greater than 99% and chemical purity was greater than 95%. [¹¹C]SIL5 was identified by co-eluting with the solution of standard SIL5. The entire synthetic procedure including the production of [¹¹C]CH₃I, radiosynthesis, HPLC purification and formulation of the radiotracer for vivo studies, was completed within 50-60 min. [¹¹C]SIL5 was obtained in a specific activity of >1500 mCi/gmol at EOS (n=4).

The radiosynthesis of [¹⁸F]SIL26 was achieved using a three-step reaction. The radioactive intermediate [¹⁸F]fluoroethyltosylate ([¹⁸F]FEOTs) was first synthesized through a typical fluorination of the di-tosylate substrate. Treatment of ethylene glycol ditosylate with [¹⁸F]fluoride, potassium carbonate and Kryptofix 222 gave [¹⁸F]FEOTs in good yield (60-70%, decay corrected) after HPLC purification. The intermediate was reacted with the precursor, followed by DBU hydrolysis to give sufficiently dose of [¹⁸F]SIL26 with the labeling yield of 55-65% after HPLC purification (n=2, decay corrected). The radiochemical purity of [¹⁸F]SIL26 was greater than 98% and chemical purity was greater than 95%. [¹⁸F]SIL26 was identified by co-eluting with the solution of standard SIL26. The entire synthetic procedure including the drying of [¹⁸F]F⁻, the radiosynthesis, HPLC purification and formulation of the radiotracer for vivo studies, was completed in 3 hr. [¹⁸F]SIL26 was obtained in a specific activity of >2000 mCi/gmol (decay corrected to EOB, n=2).

### Example 38

### Ex Vivo Biodistribution of [¹¹C]SIL5 and [¹⁸F]SIL26 in Sprague Dawley Rats

Ex vivo biodistribution studies of [¹¹C]SIL5 and [¹⁸F]SIL26 in Sprague Dawley rats were performed to determine whether [¹¹C]SIL5 and [¹⁸F]SIL26 are able to penetrate the blood-brain-barrier (BBB) and have sufficient brain uptake and quick washout from the brain of control rats.

For the biodistribution studies, 300-350 μCi of [¹¹C]SIL5 in 200-250 μL, or 45-50 μCi of [¹⁸F]SIL26 in 200-250 μL of saline containing 10% ethanol was injected via the tail vein into mature male Sprague-Dawley rats (175-240 g) under 2-3% isoflurane/oxygen anesthesia. Group of rats (n=4) were used for each time point. At 5, 30, 60 min (and 120 min for [¹⁸F]SIL26) post intraveneous injection, the rats were anesthetized and euthanized. The whole brain was quickly removed and dissected into segments consisting of brain stem, thalamus, striatum, hippocampus, cortex, and cerebellum. The remainder of the brain was also collected to determine total brain uptake. At the same time, samples of blood, heart, lung, muscle, fat, pancreas, spleen, kidney, liver (and bone for [¹⁸F]SIL26) were removed and counted in a Beckman Gamma 8000 well counter with a standard dilution of the injectate. Tissues were weighed, and the % I.D./g for each tissue was calculated.

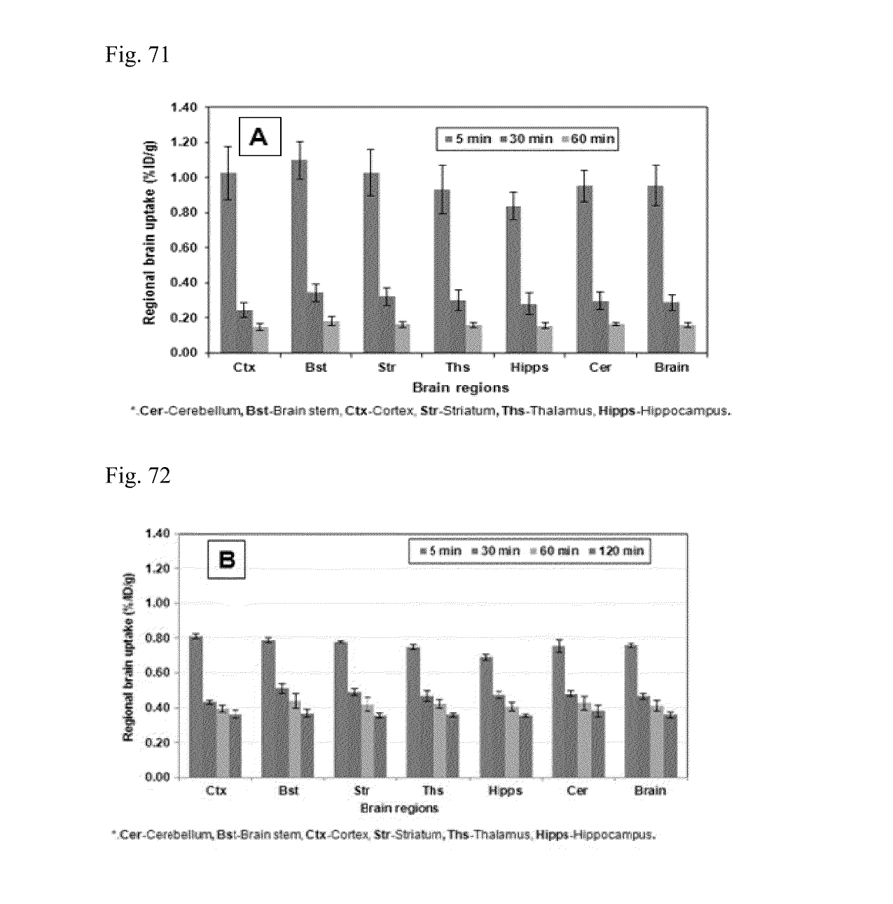

The radioactivity distribution in various organs after injection of [¹¹C]SIL5 and [¹⁸F]SIL26 in rats is summarized in Table 7. Both radiotracers displayed homogeneous distribution in the brain regions as shown in FIGS. 71 and 72. For [¹¹C]SIL5, the brain uptake (% I.D./g) of radioactivity at 5, 30 and 60 min post injection were 0.953±0.115, 0.287±0.046, and 0.158±0.014 respectively; in the peripheral tissue, liver has the highest amount of uptake among the tissues that were analyzed; the uptake (% I.D./g) in liver reached 2.198±0.111 at 5 min and 1.116±0.024 at 60 min; it decreased about 50% from 5 min to 60 min. For [¹⁸F]SIL26 the brain uptake (% I.D./g) at 5, 30, 60 and 120 min were 0.758±0.013, 0.465±0.018, 0.410±0.030, and 0.359±0.016 respectively; of the peripheral tissues analyzed, this compound also has the highest liver uptake (% I.D./g) of 1.626±0.221 at 5 min, which is similar to that of [¹¹C]SIL5. However, after 30 min, the kidney has retained the highest uptake of all tissues that were analyzed. From 5 to 60 min, the bone uptake (% I.D./g) was very steady and no defluorination was observed for [¹⁸F]SIL26. At 120 min, bone uptake has slight increase to 0.644±0.071 (% I.D./g) compare to the bone uptake at 60 min; considering the variability of studies, the defluorination should not be a concern for radiotracer [¹⁸F]SIL26. More importantly, the ex vivo data revealed that both compounds can easily cross the BBB and enter the brain. Both tracers exhibit high initial brain uptake and appropriate washout kinetics. Rapid clearance of the radioactivity for both [¹¹C]SIL5 and [¹⁸F]SIL26 was observed from the brain as well as body organs such as lung, pancreas, spleen, kidney and liver. However, [¹¹C]SIL5 showed faster wash-out trend than [¹⁸F]SIL26, as shown in FIGS. 71 and 72.

TABLE 7
Biodistribution of [¹¹C]SIL5 and [¹⁸F]SIL26
in male Sprague-Dawley rats (I.D. %/gram)

| Radioligand | Organ | 5 min | 30 min | 60 min | 120 min |
|---|---|---|---|---|---|
| [¹¹C]SIL5 | blood | 0.506 ± 0.040 | 0.369 ± 0.031 | 0.300 ± 0.015 | |
| | heart | 0.758 ± 0.052 | 0.377 ± 0.046 | 0.245 ± 0.010 | |
| | lung | 1.149 ± 0.058 | 0.740 ± 0.038 | 0.485 ± 0.036 | |
| | muscle | 0.271 ± 0.005 | 0.325 ± 0.030 | 0.199 ± 0.016 | |
| | fat | 0.155 ± 0.023 | 0.241 ± 0.042 | 0.293 ± 0.076 | |
| | pancreas | 1.007 ± 0.262 | 0.506 ± 0.066 | 0.522 ± 0.036 | |
| | spleen | 0.659 ± 0.049 | 0.400 ± 0.052 | 0.386 ± 0.027 | |
| | kidney | 1.362 ± 0.054 | 0.807 ± 0.086 | 0.559 ± 0.053 | |
| | liver | 2.198 ± 0.111 | 1.349 ± 0.116 | 1.116 ± 0.024 | |
| [¹⁸F]SIL26 | blood | 0.553 ± 0.047 | 0.589 ± 0.016 | 0.606 ± 0.035 | 0.585 ± 0.046 |
| | heart | 0.757 ± 0.033 | 0.505 ± 0.015 | 0.466 ± 0.040 | 0.410 ± 0.030 |
| | lung | 0.833 ± 0.053 | 0.561 ± 0.021 | 0.491 ± 0.030 | 0.436 ± 0.018 |
| | muscle | 0.430 ± 0.031 | 0.451 ± 0.018 | 0.376 ± 0.019 | 0.315 ± 0.015 |
| | fat | 0.255 ± 0.037 | 0.425 ± 0.023 | 0.371 ± 0.067 | 0.293 ± 0.048 |
| | pancreas | 1.004 ± 0.147 | 0.546 ± 0.068 | 0.409 ± 0.029 | 0.330 ± 0.021 |
| | spleen | 0.672 ± 0.064 | 0.509 ± 0.022 | 0.446 ± 0.038 | 0.398 ± 0.019 |
| | kidney | 1.070 ± 0.058 | 0.988 ± 0.090 | 0.678 ± 0.032 | 0.659 ± 0.027 |
| | liver | 1.626 ± 0.221 | 0.847 ± 0.027 | 0.561 ± 0.028 | 0.467 ± 0.023 |
| | bone | 0.340 ± 0.027 | 0.309 ± 0.020 | 0.407 ± 0.043 | 0.644 ± 0.071 |

In summary, two potent α-synuclein ligands, [¹¹C]SIL5 and [¹⁸F]SIL26 were successfully radiosynthesized by O-alkylation of the desalkyl precursor. The biodistribution studies of [¹¹C]SIL5 and [¹⁸F]SIL26 were conducted in male Sprague-Dawley rats and found both tracers were able to cross BBB and enter into the brain and have high initial uptake; At 5 min, the uptake (% I.D./g) reached 0.953±0.115 for [¹¹C]SIL5 and 0.758±0.013 for [¹⁸F]SIL26. In the control rats, both [¹¹C]SIL5 and [¹⁸F]SIL26 displayed homogeneous distribution in the brain regions of interest, have quick washout kinetics from the brain.

### Example 39

### MicroPET Study of Radioligand [¹¹C]SIL5 in the Brain of a Nonhuman Primate

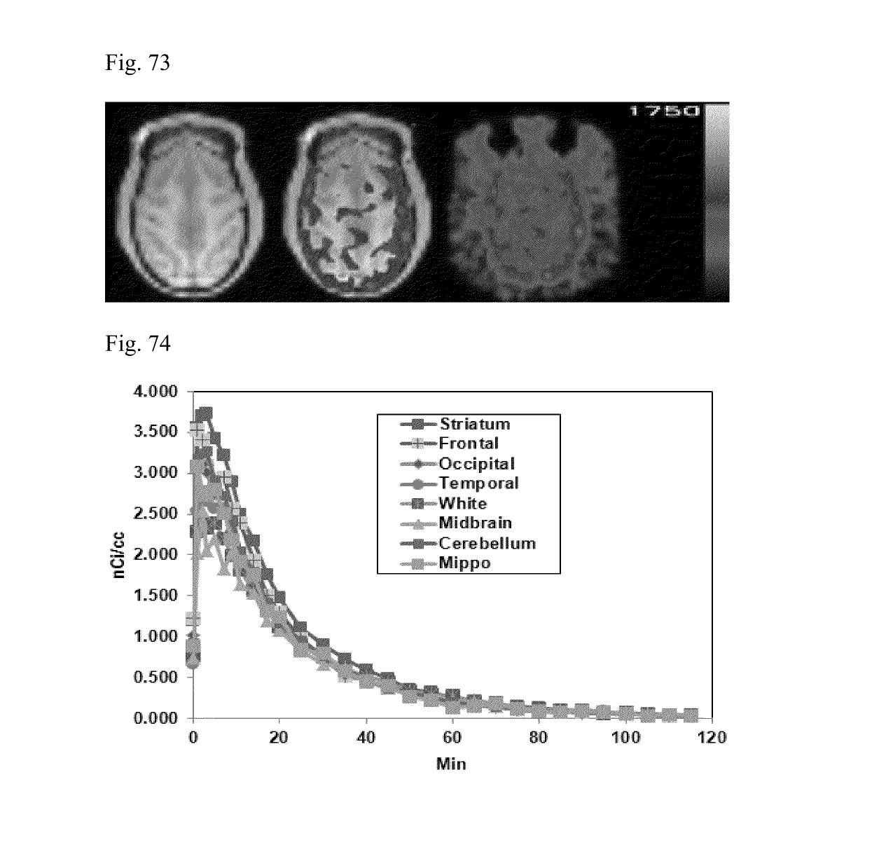

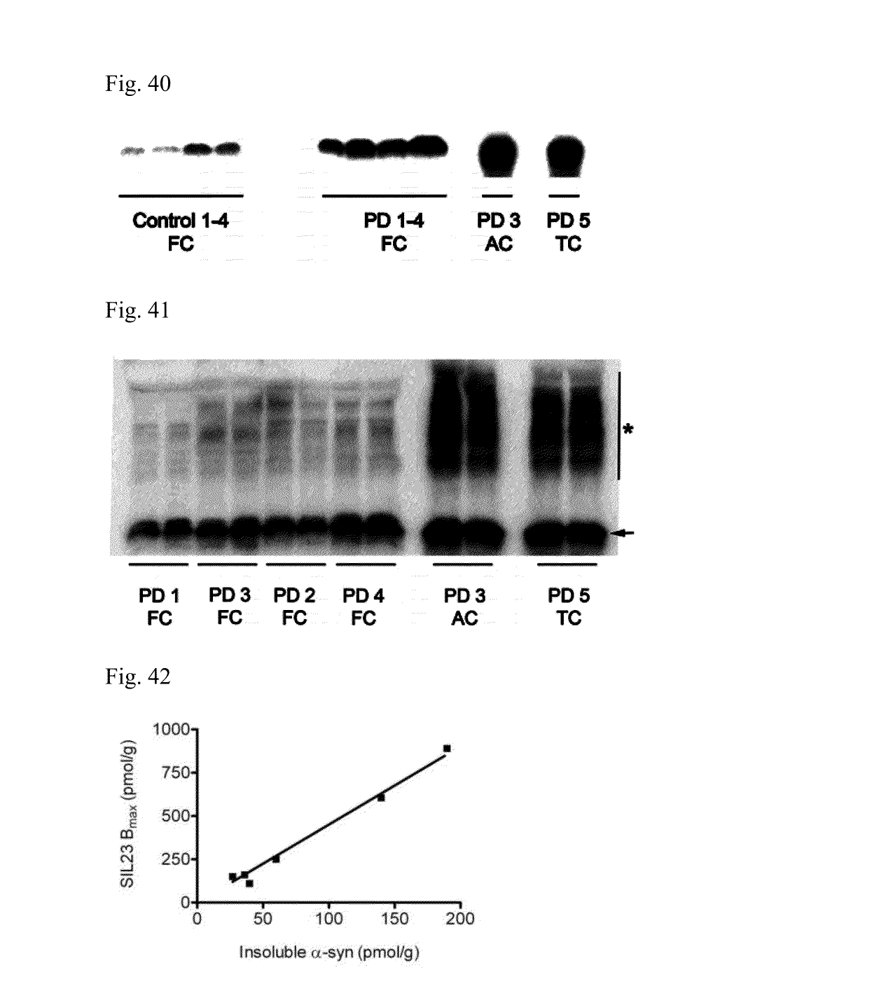

A monkey weighing 5.8 kg was injected with 8.68 mCi/6 mL of radioligand [¹¹C]SIL5. The brain of a cynologmus monkey was scanned using a microPET scanner. An image of the scan is provided in FIG. 73. The left panel is the MRI image. The middle panel is the co-registered the MRI and PET image. The right panel is the PET image. The images show that the [¹¹C]SIL5 displayed high uptake in the brain and has homogenetic distribution in the brain. FIG. 74 presents a plot of the radiation in portions of the monkey brain as a function of time. The Figure shows that the compound has the capability of entering into the brain and has homogenetic distribution in the brain. It also has favorable clearance pharmacokinetics from the brain. Accordingly, this experiment indications that radioligand [¹¹C]SIL5 entered the monkey's brain and has good washout kinetics.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above compositions and methods without departing from the scope of the invention, it is intended that all matter contained in the above description and shown in the accompanying figures shall be interpreted as illustrative and not in a limiting sense.

Having described the invention in detail, it will be apparent that modifications and variations are possible without departing from the scope of the invention defined in the appended claims.

### REFERENCES

The content of the following references are hereby incorporated herein by reference for all relevant purposes.
1. Masuda et al., “Small Molecule Inhibitors of α-Synuclein Filament Assembly,” 2006, Biochemistry, 45(19): 6085-6094.
2. Massie, S., “The Chemistry of Phenothiazine,” 1954, Chem Reviews, Vol. 54 (5), 797-833.
3. González-Munoz et al., “Old phenothiazine and dibenzothiadiazepine derivatives for tomorrow's neuroprotective therapies against neurodegenerative diseases,” 2010, Eur J Med Chem, 45(12): 6152-6158.
4. Tu et al., “Radiosynthesis and in vivo evaluation of [¹¹C]MP-10 as a PET probe for imaging PDE10A in rodent and non-human primate brain,” 2011, Bioorg. Med. Chem. 19: 1666-1673.
5. Tu et al., “Carbon-11 labeled papaverine as a PET tracer for imaging PDE10A: radiosynthesis, in vitro and in vivo evaluation,” 2010, Nuclear Medicine and Biology 37:509-516.
6. Hughes, A. J., et al, “Accuracy of clinical diagnosis of idiopathic Parkinson's disease: a clinico-pathological study of 100 cases,” 1992, J Neurol Neurosurg Psychiatry, 55:181-184.
7. Galvin, J. E., et al., “The ADB: a brief informant interview to detect dementia,” 2005, Neurology 65:559-564.
8. Folstein, M. F., et al., ““Mini-mental state”. A practical method for grading the cognitive state of patients for the clinician,” 1975, J Psychiatr Res, 12:189-198.
9. Braak, H., et al., “Stages in the development of Parkinson's disease-related pathology,” 2004, Cell Tissue Res 318:121-134.
10. Giasson, B. I., et al., “Neuronal alpha-synucleinopathy with severe movement disorder in mice expressing A53T human alpha-synuclein,” 2002, Neuron, 34:521-533.
11. Giasson, B. I., et al., “Mutant and wild type human alpha-synucleins assemble into elongated filaments with distinct morphologies in vitro,” 1999, J Biol Chem, 274:7619-7622.
12. Huang, C., et al., “A new method for purification of recombinant human alpha-synuclein in Escherichia coli,” 2005, Protein Expr Purif, 42:173-177.
13. Yu, L., et al, “Synthesis and in vitro evaluation of d-synuclein ligands,” 2012, Bioorg Med Chem in press.
14. Li, W., et al., “Characterization of two VQIXXK motifs for tau fibrillization in vitro,” 2006, Biochemistry, 45:15692-15701.
15. Frost, B., et al, “Propagation of tau misfolding from the outside to the inside of a cell,” 2009, J Biol Chem 284: 2845-12852.
16. Jan, A., et al., “Preparation and characterization of toxic Abeta aggregates for structural and functional studies in Alzheimer's disease research,” 2010, Nat Protoc, 5:1186-1209. [DOI] nprot.2010.72 [pii];10.1038/nprot.2010.72.
17. Kotzbauer, P. T., et al., “Fibrillization of alpha-synuclein and tau in familial Parkinson's disease caused by the A53T alpha-synuclein mutation,” 2004, Exp Neurol, 187:279-288.
18. Engel, L. A., et al., “Catalytic function of PLA2G6 is impaired by mutations associated with infantile neuroaxonal dystrophy but not dystonia-parkinsonism,” 2010, PLoS ONE 5: e12897.
19. Giasson, B. I., “Oxidative damage linked to neurodegeneration by selective alpha-synuclein nitration in synucleinopathy lesions,” 2000, Science, 290:985-989. 8948 [pii].
20. Klunk, W. E., et al., “Binding of the positron emission tomography tracer Pittsburgh compound-B reflects the amount of amyloid-beta in Alzheimer's disease brain but not in transgenic mouse brain,” 2005, J Neurosci, 25:10598-10606.
21. Fodero-Tavoletti, M. T., et al., “In vitro characterisation of BF227 binding to alpha-synuclein/Lewy bodies,” 2009, Eur J Pharmacol, 617:54-58.
22. Fodero-Tavoletti, M. T., et al., “In vitro characterization of Pittsburgh compound-B binding to Lewy bodies,” 2007, J Neurosci, 27:10365-10371.
23. Ye L., et al., “In vitro high affinity alpha-synuclein binding sites for the amyloid imaging agent PIB are not matched by binding to Lewy bodies in postmortem human brain,” 2008, J Neurochem, 105:1428-1437.
24. Choi, S. R., et al., “Preclinical properties of 18F-AV-45: a PET agent for Abeta plaques in the brain,” 2009, J Nucl Med, 50:1887-1894.
25. Chakrabarty, M., et al., “Dbu, a highly efficient reagent for the facile regeneration of (hetero)arylamines from their acetamides and benzamides: Influence of solvent, temperature, and microwave irradiation,” 2002, Synth. Commun, 32:265-272.

1. A compound of Formula (I) 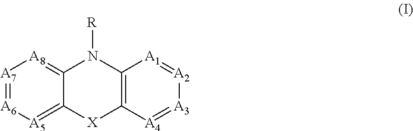
wherein X is oxygen or sulfur;
R is hydrogen, alkyl, or acyl;
A₁ is C—R₁ or nitrogen;
A₂ is C—R₂ or nitrogen;
A₃ is C—R₃ or nitrogen;
A₄ is C—R₄ or nitrogen;
A₅ is C—R₅ or nitrogen;
A₆ is C—R₆ or nitrogen;
A₇ is C—R₇ or nitrogen;
A₈ is C—R₈ or nitrogen; and
R₁, R₂, R₃, R₄, R₅, R₆, R₇, and R₈ are each independently hydrogen, halo, hydroxy, substituted or unsubstituted alkyl, substituted or unsubstituted alkoxy, cyano, nitro, amino, alkylamino, or dialkylamino; or a pharmaceutically acceptable salt thereof.

2. (canceled)

3. (canceled)

4. The compound of claim 1 wherein R is hydrogen, C₁-C₆ alkyl, or C₁-C₆ acyl.

5. The compound of claim 4 wherein R is hydrogen, methyl, or acetyl.

6. (canceled)

7. The compound of claim 1 wherein one or more of A₁, A₂, A₃, A₄, A₅, A₆, A₇, or A₈ is nitrogen.

8. The compound of claim 7 wherein either A₁ or A₃ is nitrogen and A₂ is C—R₂, A₄ is C—R₄, A₅ is C—R₅, A₆ is C—R₆, A₇ is C—R₇, and A_{g} is C—R₈.

9. The compound of claim 1, wherein A₁ is C—R₁, A₂ is C—R₂, A₃ is C—R₃, A₄ is C—R₄, A₅ is C—R₅, A₆ is C—R₆, A₇ is C—R₇, and A₈ is C—R₈.

10. The compound of claim 1 wherein R₁, R₂, R₃, R₄, R₅, R₆, R₇, and R₈ are each independently hydrogen, fluoro, bromo, iodo, hydroxy, substituted or unsubstituted C₁-C₆ alkyl, substituted or unsubstituted C₁-C₆ alkoxy, cyano, nitro, amino, C₁-C₆ alkylamino, or di-C₁-C₆ alkylamino.

11. The compound of claim 10 wherein R₁, R₂, R₃, R₄, R₅, R₆, R₇, and R₈ are each independently hydrogen, bromo, iodo, hydroxy, C₁-C₄ alkyl, C₁-C₄ haloalkyl, substituted or unsubstituted C₁-C₄ alkoxy, cyano, nitro, amino, C₁-C₄ alkylamino, or di-C₁-C₄ alkylamino.

12. (canceled)

13. The compound of claim 11 wherein R₁, R₂, R₃, R₄, R₅, R₆, R₇, and R₈ are each independently hydrogen, bromo, iodo, hydroxy, methyl, trifluoromethyl, methoxy, propargyloxy (i.e., —OCH₂C≡CH), 2-fluoroethoxy (i.e., —OCH₂CH₂F), 3-iodoallyloxy (i.e., —OCH₂CH═CHI), cyano, nitro, amino, methylamino, or dimethylamino.

14. (canceled)

15. The compound of claim 1 wherein at least one of R₁, R₂, R₃, R₄, R₅, R₆, R₇, and R₈ is methoxy, nitro, bromo, or iodo.

16-21. (canceled)

22. The compound of claim 1 wherein at least one of R₁, R₂, R₃, R₄, R₅, R₆, R₇, and R₈ is nitro and at least one of R₁, R₂, R₃, R₄, R₅, R₆, R₇, and R₈ is methoxy.

23. The compound of claim 1 wherein at least one of R₁, R₂, R₃, R₄, R₅, R₆, R₇, and R₈ is nitro and at least one of R₁, R₂, R₃, R₄, R₅, R₆, R₇, and R₈ is bromo or iodo.

24. The compound of claim 1, wherein the compound of Formula I is selected from the group consisting of: 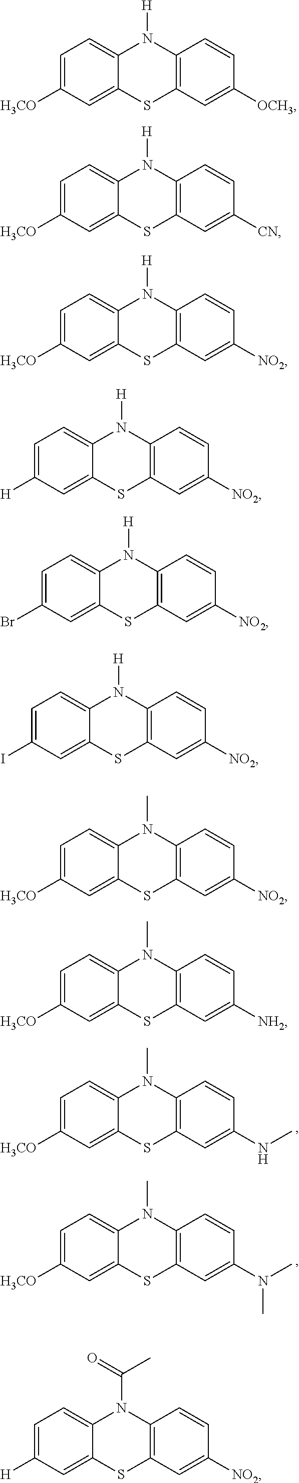 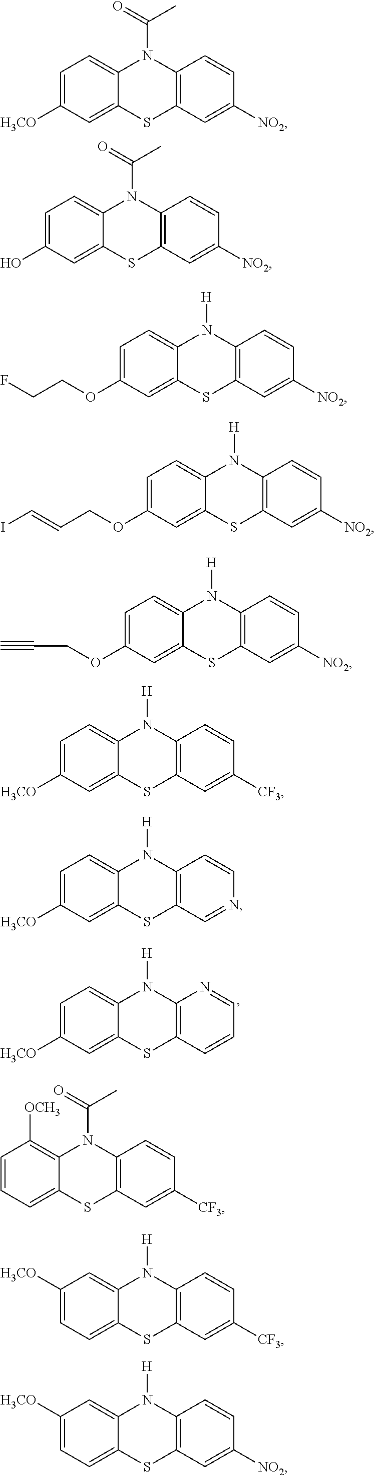 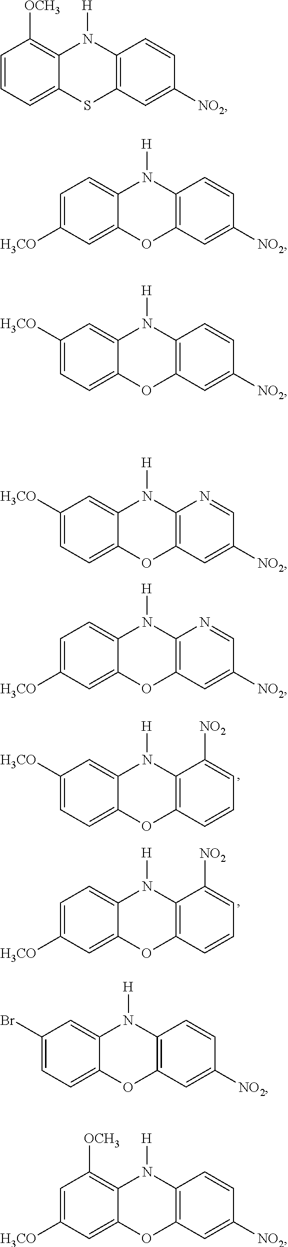
and pharmaceutically acceptable salts thereof.

25. The compound of claim 1 wherein the compound is radiolabeled with an isotope.

26. The compound of claim 25 wherein the isotope is selected from the group consisting of carbon-11, nitrogen-13, oxygen-15, fluorine-18, bromine-76, iodine-123, and iodine-125.

27. A method for diagnosing or monitoring a synucleinopathy in a human subject comprising:
administering a radiolabeled compound of claim 25 to the human subject; and
imaging the subject's brain by positron emission tomography.

28. The method of claim 27 wherein the synucleinopathy is Parkinson's disease, Dementia with Lewy Bodies, or multiple system atrophy.

29. A method of treating a synucleinopathy in a human subject in need thereof comprising administering a therapeutically effective amount a compound of claim 1 to the human subject.

30. The method of claim 29 wherein the synucleinopathy comprises Parkinson's Disease, Dementia with Lewy Bodies, or multiple system atrophy.

31. A method for determining the binding affinity of a compound to α-synuclein fibrils comprising:
preparing a plurality of test mixtures comprising α-synuclein fibrils, Thioflavin T (ThT) and a test compound, wherein the test mixtures contain varied concentrations of the test compound;
incubating the test mixtures;
measuring a fluorescence intensity of each test mixture at the maximum fluorescence emission wavelength and excitation wavelength of ThT; and
determining the amount of ThT inhibited from binding to α-synuclein fibrils for each test mixture.

32. (canceled)
